(19) United States
(12) Patent Application Publication
(10) Pub. No.: US 2013/0130265 A1
(43) Pub. Date: May 23, 2013

(54) METHODS AND DEVICES FOR OBTAINING AND ANALYZING CELLS

(71) Applicants: Bhairavi PARIKH, Palo Alto, CA (US); Michael D. BRODY, Fremont, CA (US); James STONE, Saratoga, CA (US); Brian AWABDY, Pleasanton, CA (US); Urvi VED, Fremont, CA (US); Anh TRAN, Tracy, CA (US); David J. ANVAR, Sunnyvale, CA (US)

(72) Inventors: Bhairavi PARIKH, Palo Alto, CA (US); Michael D. BRODY, Fremont, CA (US); James STONE, Saratoga, CA (US); Brian AWABDY, Pleasanton, CA (US); Urvi VED, Fremont, CA (US); Anh TRAN, Tracy, CA (US); David J. ANVAR, Sunnyvale, CA (US)

(21) Appl. No.: 13/681,296

(22) Filed: Nov. 19, 2012

Related U.S. Application Data
(60) Provisional application No. 61/561,231, filed on Nov. 17, 2011.

(51) Int. Cl. C12Q 1/68

(52) U.S. Cl. CPC C12Q 1/686
USPC 435/6.12; 435/180

(57) ABSTRACT

A method for concentrating and isolating nucleated cells, such as maternal and fetal nucleated red blood cells (nRBCs), in a maternal whole blood sample. The invention also provides methods and apparatus for preparing to analyze and analyzing the sample for identification of fetal genetic material as part of prenatal genetic testing. The invention also pertains to methods and apparatus for discriminating fetal nucleated red blood cells from maternal nucleated red blood cells obtained from a blood sample taken from a pregnant woman.

### CROSS REFERENCE TO RELATED APPLICATIONS

This application claims the benefit under 35 U.S.C. §119 of U.S. Patent Application No. 61/561,231 filed Nov. 17, 2011, the disclosure of which is incorporated herein by reference

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Prenatal genetic testing requires access to fetal DNA. As described in commonly owned US Patent Appl. Publ. No. 2010/0159506, fetal genetic material can be found within fetal cells present in the mother's circulating blood. These fetal cells originate in the fetus and cross the placenta to enter the mother's circulatory system.

The most common types of fetal cells in maternal circulation are blood cells. CFCs (circulating fetal cells) are a heterogenous group of cells including trophoblasts, leukocytes, nucleated erythrocytes and other stem and progenitor cells. Not all CFCs are useful for genetic testing of current pregnancies. Trophoblast isolation and enrichment may be impeded by their multinucleated morphology and the limited availability of antibodies specific to placental antigens, while certain leukocytes may persist from previous pregnancies and lack unique cell markers or HLA antigens to differentiate maternal from fetal leukocytes.

The most attractive CFCs for analysis are fetal nucleated red bloods (fnRBCs), which have a short half-life (˜30 days) and are relatively abundant throughout pregnancy. But, the reliable detection and isolation of fnRBCs poses a significant technical challenge as they are very rare and nRBCs can be fetal or maternal.

The approximate concentration of fnRBCs in whole blood is approximately 1:1,000,000,000. Direct analysis of whole blood for rare events is too slow and expensive to support some medical diagnostic applications.

Thus there is a need in the field of prenatal genetic testing to create a new and useful method and system for reliably detecting and isolating fnRBCs. This invention provides such a new and useful method and system.

### SUMMARY OF THE INVENTION

Provided herein are methods, devices, systems and kits for obtaining, identifying, and analyzing cells and material such as, for example, nucleic acids (e.g. DNA), organelles, membranes, and proteins that can be obtained from a cell. The methods, devices, systems, and kits may be especially useful to obtain or analyze single cells and/or rare cells. Some particular cells that may be obtained and analyzed include fetal cells from a maternal blood sample, circulating tumor cells, stem cells, nucleated red blood cells and other rare cells from a blood sample.

The present invention provides a method for concentrating and isolating nucleated cells, such as maternal and fetal nucleated red blood cells (nRBCs), from a maternal whole blood sample. The invention also provides methods and apparatus for preparing to analyze and analyzing the sample for identification of fetal genetic material as part of prenatal genetic testing. The invention also pertains to methods and apparatus for discriminating fetal nucleated red blood cells from maternal nucleated red blood cells obtained from a blood sample taken from a pregnant woman.

One aspect of the invention provides a method of enriching for fetal nucleated cells (such as, e.g. nucleated red blood cells) from a maternal blood sample. In some embodiments, the method includes the following steps: passing a maternal blood sample containing maternal nucleated cells and fetal nucleated cells through a filter (such as, e.g., a leukocyte depletion filter); retaining nucleated cells on the filter; and eluting nucleated cells from the filter with an elution buffer wherein the nucleated cells include fetal nucleated cells.

In some embodiments, the method also includes the step of staining the blood sample, such as by staining the blood sample with a nuclear stain.

In some embodiments, the method also includes the step of concentrating and resuspending the blood sample before the passing step, such as by centrifugation.

In some embodiments, the method also includes the step of measuring the blood sample before concentrating to, e.g., standardize an amount of cells in the blood sample.

Another aspect of the invention provides a method of creating a layer of cells on a surface including the following steps: moving a sample of cells in at least two directions relative to a surface to create a monolayer of cells on the surface; and adhering the cells to the surface. The movement may include, e.g., circular movement, zigzag movement, diagonal movement and/or serpentine movement. The relative movement may also include moving a portion of the sample away from the surface.

Some embodiments employ a smear tool in the moving step. In some such embodiments, the angle of the tool with respect to the surface may be varied. The relative speed of the tool with respect to the surface may also be varied, for example, in the range of 0.1 mm/sec to 500 mm/sec.

The moving step may include the step of generating a generally uniform sample density. Some embodiments also include the step of monitoring the density of the sample relative to the surface, such as by using red or blue light.

Yet another aspect of the invention provides a method of identifying a nucleated fetal cell. In some embodiments the method includes the steps of: adhering nucleated cells from a maternal blood sample to a surface, the surface comprising a plurality of portions; generating a pair of images corresponding to at least one portion of the surface; applying an algorithm to the pair and determining if the portion includes a cell of interest; and performing an analysis using a fetal identifier on at least one portion of the surface that includes a cell of interest and thereby determine if the at least one portion contains a fetal cell. In some embodiments, the analysis is selected from the group consisting of in situ hybridization and immunohistochemistry, and the method further includes the step of scanning the surface with an automated microscope after the adhering step. Some embodiments include the step of generating a third image.

In some embodiments, the step of generating a pair of images further includes the step of generating a first image with transmitted illumination and a second image with coaxial illumination. In various embodiments the wavelength of the transmitted illumination may be between 380 nm and 800 nm, above 620 nm, or around 420 nm. The coaxial illumination may be between 350 nm and 364 nm.

In some embodiments, the step of performing an analysis includes the step of selectively placing fetal identifiers on a plurality of portions of the surface, wherein each portion contains a candidate fetal cell.

In some embodiments, the step of applying an algorithm includes the steps of flattening at least one image; segmenting at least one image and thereby define foreground and background pixels; removing background pixels from at least one image to generate a transformed image; enumerating nuclei in the transformed image to generate enumerated nuclei; and calculating at least one of complexity and brightness for at least one enumerated nuclei, wherein low complexity or high brightness indicate a fetal cell character.

In some embodiments, the applying step further includes the steps of calculating a brightness of a background of the surface and a brightness of a foreground of a surface, and comparing a measurement of the pair of images to one or both of the background brightness and the foreground brightness.

Some embodiments include the additional step of storing a location of the pair of images. A nucleated cell may be located based on such a stored location.

Some embodiments include the step of fixing the sample with a non-cross linking fixative before the performing an analysis step and/or fixing the sample at a reduced temperature before the performing an analysis step.

In some embodiments, the performing step may include the step of treating with a stabilizer and/or treating with an antibody selected from the group consisting of anti-zeta hemoglobin, anti-gamma, and anti-epsilon hemoglobin if the analysis is immunohistochemistry.

Still another aspect of the invention provides a method of identifying a genetic status of a fetus. In some embodiments the method includes the following steps: adhering nucleated cells from a maternal blood sample to a surface, the surface comprising a plurality of portions; generating a pair of images corresponding to at least one portion; applying an algorithm to the pair of images and determining if the portion includes a cell of interest; performing an analysis using a fetal identifier on at least one portion of the surface that includes a cell of interest and thereby determine if the at least one portion contains a fetal cell (such as, e.g., by in situ hybridization and/or immunohistochemistry); and performing an analysis using a genetic identifier on at least one portion of the surface that includes a fetal cell and thereby determine the genetic status of the fetus (such as, e.g., by RNA in situ hybridization, DNA in situ hybridization and/or immunohistochemistry). The two performing steps may be performed at the same time.

Some embodiments may include the additional step of applying a pressure on the surface that is lower than atmospheric pressure during the performing step(s). The method may also include the step of crushing at least one cell of interest and an associated nuclei prior to the performing the analysis step.

Yet another aspect of the invention provides a method of identifying a fetal cell. In some embodiments, the method includes the following steps: providing a maternal blood sample; and performing in situ hybridization using a TSIX probe on the sample to generate a signal wherein a positive signal using a TSIX probe is indicative of the presence of fetal cellular material. The method may also include the step of separating the sample to generate a plurality of portions before the performing step to, e.g., provide a monolayer of cells.

Another aspect of the method provides a method of determining a genetic status of a fetus. In some embodiments, the method includes the steps of providing a maternal blood sample comprising maternal cells and a fetal cell; self-assembling a monolayer of cells from the maternal blood sample on a surface; performing a first analysis on the monolayer to thereby distinguish a potential fetal cell in the monolayer; selectively removing the potential fetal cell from the monolayer; obtaining DNA from the potential fetal cell; analyzing a characteristic of the DNA to obtain a signal based on the DNA; and comparing the signal with a reference characteristic to thereby determine a genetic status of the potential fetal cell.

In some embodiments, the method further includes enriching the maternal blood sample to thereby generate a sample enriched in nucleated cells and a fetal cell prior to the self-assembling step. In some embodiments, the method further includes enriching the maternal blood sample to thereby generate a sample enriched in maternal white blood cells and a fetal cell prior to the self-assembling step. In some embodiments, the method further includes enriching the maternal blood sample to thereby generate a sample having between a 5000:1 to a 1:10 ratio of red blood cells to white blood cells and a fetal cell prior to the self-assembling step.

In some embodiments wherein the maternal blood sample includes viable cells, the step of self-assembling a monolayer of cells includes self-assembling a monolayer comprising viable cells. In some such embodiments, the method further includes maintaining the viability of the cells at least through the selectively removing step.

In some embodiments, the step of self-assembling a monolayer of cells includes self-assembling a monolayer of cells wherein a first portion of the cells are immobilized with respect to one another. In some embodiments, the step of self-assembling a monolayer of cells comprises removably attaching a first portion of the cells to the surface. In some embodiments, the step of self-assembling a monolayer of cells includes settling a second portion of the cells close to the surface. In some such embodiments, the method includes the step of maintaining a second portion of cells close to the surface. In some such embodiments, maintaining includes maintaining a second portion of cells close to the surface for at least 15 minutes, at least 30 minutes, at least 45 minutes, or at least one hour.

In some embodiments, method further includes the step of treating the cells with an identifier. In some embodiments, the method includes the step of treating the cells with a nuclear identifier prior to the performing a first analysis step, wherein performing a first analysis comprises analyzing the monolayer of cells to detect a cell positive for the nuclear identifier. In some such embodiments wherein the nuclear identifier includes SYBR Green, performing a first analysis includes the step of illuminating the cells with a SYBR Green excitation light and detecting a SYBR Green emission light from the cells. In some such embodiments, the step of illuminating includes the step of illuminating the cells with a blue light and the step of detecting includes the step of detecting a green light.

In some embodiments, the step of performing a first analysis on the monolayer of cells includes the step of detecting hemoglobin. In some embodiments, the step of detecting hemoglobin includes the steps of illuminating the monolayer of cells with a hemoglobin absorbable light (such as, e.g. with a wavelength between 400 nm and 600 nm or between 400 nm to 450 nm) and detecting light from the monolayer of cells based on hemoglobin light absorption. In some embodiments, the step of illuminating the monolayer of cells includes the step of diffusing the light.

In some embodiments, the step of analyzing the monolayer includes the step of detecting hemoglobin wherein a cell positive for a nuclear identifier and positive for hemoglobin identifies a candidate fetal cell.

In some embodiments, the method further includes the steps of fixing the cells on the surface and analyzing the cells using at least one of immunocytochemistry and in situ hybridization. In some such embodiments wherein the method includes immunocytochemistry, the method further include the step of analyzing using an anti-hemoglobin antibody to thereby detect a cell containing hemoglobin.

In some embodiments, the method further includes the steps of placing the potential fetal cell on a substrate after the selectively removing step; and performing a second analysis on the potential fetal cell on the substrate to confirm fetal cell identity. In some such embodiments, the performing a second analysis includes performing immunocytochemistry. In some such embodiments, performing immunocytochemistry includes the steps of performing immunocytochemistry with an anti-hemoglobin antibody (such as, e.g. using at least one of an anti-fetal hemoglobin antibody and an anti-embryonic antibody) and obtaining an immunocytochemical signal to thereby confirm candidate fetal cell identity. In some of these embodiments, the method further includes the steps of thresholding a value of the immunocytochemical signal wherein a signal value above a first threshold value, a signal value below a second threshold value, or both, identifies a candidate fetal cell.

In some embodiments, the method further includes the step of amplifying DNA (e.g. using polymerase chain reaction (PCR)) from the potential fetal cell to generate first amplified DNA wherein analyzing comprises obtaining a signal based on the first amplified DNA. In some of these embodiments, the method further includes analyzing using least one of quantitative PCR (qPCR) and digital PCR (dPCR). In some embodiments, the amplifying using PCR includes amplifying for less than 6 cycles to generate first amplified DNA. In some embodiments, analyzing the DNA includes analyzing based on a fetal identifier wherein a positive signal based first amplified DNA identifies a fetal cell. In some such embodiments wherein the fetal identifier includes a short tandem repeat (STR) and the step of analyzing the DNA includes the step of analyzing the short tandem repeat.

In some embodiments, the step of performing further includes distinguishing a plurality of potential fetal cells (e.g. such as from the same monolayer on a surface or from monolayers on a plurality of surfaces), amplifying further comprises amplifying a first portion of DNA for less than 6 cycles from each potential fetal cells of the plurality of cells to obtain a plurality of DNA samples, analyzing further comprising analyzing the plurality of DNA samples based on a fetal identifier to obtain a plurality of fetal cells each having a positive signal, the method further comprising pooling and amplifying a second portion of the DNA from the plurality of fetal cells each having a positive signal to obtain pooled, amplified DNA.

Some embodiments further include the steps of dividing the DNA from the potential fetal cell into a first aliquot and a second aliquot prior to the amplifying using PCR step, amplifying the second aliquot using polymerase chain reaction (PCR) to generate second amplified DNA; analyzing a characteristic of the second amplified DNA to obtain a second signal based on the DNA; and comparing the second signal with a reference characteristic to thereby determine a second genetic characteristic of the cell. In some such embodiments wherein the step of analyzing a characteristic of the second amplified DNA includes the step of performing one of DNA sequencing and array genomic hybridization (aCGH). In some such embodiments wherein the step of comparing the signal with a reference characteristic includes the step of comparing the signal with a reference characteristic based on such as, e.g. maternal nucleic acid or a reference nucleic based on nucleic acid representing more than one individual.

In some embodiments wherein the maternal blood sample includes a plurality of fetal cells, the performing step includes the step of distinguishing a plurality of potential fetal cells, the selectively removing step includes the step of selectively removing a plurality of potential fetal cells, the obtaining step includes obtaining DNA from the plurality of cells and generating pooled DNA, wherein analyzing includes analyzing a characteristic of the pooled DNA to obtain a signal based on the pooled DNA; and comparing includes comparing the signal with a reference characteristic to thereby determine a genetic status shared by the potential fetal cells. In some such embodiments wherein the step of analyzing a characteristic of the pooled DNA includes the step of obtaining an average amplified DNA yield per cell of at least 0.35 μg when using less than 36 cycles of PCR.

In some embodiments wherein the maternal blood sample includes a plurality of fetal cells, the method further includes repeating the self-assembling, performing, and selectively removing steps to thereby obtain a plurality of potential fetal cells from at least two different cell monolayers, the obtaining step includes obtaining DNA from the plurality of cells and generating pooled DNA, wherein the analyzing step includes the step of analyzing a characteristic of the pooled DNA to obtain a signal based on the pooled DNA; and the comparing step includes the step of comparing the signal with a reference characteristic to thereby determine a genetic status shared by the potential fetal cells. In some embodiments the step of analyzing a characteristic of the pooled DNA includes the step obtaining an average amplified DNA yield per cell of at least 0.35 μg when using less than 36 cycles of PCR.

In some embodiments, the analyzing step includes sequencing at least a portion of the DNA to obtain a DNA sequence. In some embodiments, the steps of analyzing and comparing includes performing array Comparative Genomic Hybridization (aCGH) to thereby determine the genetic status of the potential fetal cell. In some embodiments, analyzing includes detecting at least one of a chromosome number, a DNA duplication, a DNA deletion, and a SNP. In some embodiments, analyzing includes detecting a chromosome number of at least one of chromosome 13, chromosome 18, chromosome 21, an X chromosome and a Y chromosome.

In some embodiments, the method further includes the steps of enriching the maternal blood sample to thereby generate a sample enriched in maternal white blood cells and at least one fetal cell having at least a 5000:1 to 1:10 ratio of red blood cells to white blood cells in the enriched maternal blood sample prior to the self-assembling step; self-assembling a viable-cell monolayer wherein a first portion of the self-assembled cells at least one of loosely attached to the surface and close to the surface; treating the cells with a live-cell nuclear identifier; optically detecting nucleated cells based on the live-cell nuclear identifier; illuminating the monolayer with a light (e.g. such as a diffuse light) between 400 nm and 600 nm and detecting light based on hemoglobin wherein cells positive for a nuclear identifier and positive for hemoglobin comprise potential fetal nucleated cells; placing the potential fetal cell on a porous substrate after the selectively removing step; repeating the selectively removing step and the placing step to thereby obtain a plurality of potential fetal cells on the porous substrate; performing a second analysis on each cell after the repeating step, the second analysis including performing immunocytochemistry using an anti-fetal hemoglobin and an anti-embryonic antibody on the plurality of potential fetal cells wherein a signal above a threshold confirms the fetal cell identity; amplifying the DNA and generating pooled amplified DNA; analyzing the DNA pool by at least one of DNA sequencing and array Comparative Genomic Hybridization to obtain a fetal DNA profile signal; and comparing the fetal DNA profile signal with a reference characteristic to thereby determine a genetic status of the fetal cells wherein the genetic status comprises at least one of aneuploidy of chromosome 13, chromosome 18, chromosome 21, an X chromosome, and a Y chromosome.

Another aspect of the invention provides a method of determining a characteristic of a fetal cell. Some embodiments include the steps of providing a maternal blood sample comprising maternal cells and a fetal cell; creating a monolayer of cells from the maternal blood sample on a surface; performing a first analysis on the monolayer of cells to thereby distinguish a potential fetal cell in the monolayer; selectively removing the potential fetal cell from the monolayer; placing the potential fetal cell on a substrate after the selectively removing step; and performing a second analysis on the cell to thereby determine a characteristic of the fetal cell.

Some embodiments further include the step of enriching the maternal blood sample to thereby generate a sample enriched in nucleated cells and a fetal cell prior to the creating step. Some embodiments further include the step of enriching the maternal blood sample to thereby generate a sample having between a 5000:1 to a 1:10 ratio of red blood cells to white blood cells and a fetal cell prior to the creating step.

In some embodiments wherein the maternal blood sample includes viable cells, the step of creating a monolayer of cells includes the step of creating a monolayer including viable cells. In some embodiments the step of creating a monolayer of cells includes the step of creating a monolayer of cells wherein a first portion of the cells are immobilized with respect to one another. In some embodiments the step of creating a monolayer of cells includes creating a monolayer of cells wherein a first portion of the cells are removably attached to the surface. Some such embodiments further include the step of settling a second portion of the cells close to the surface.

In some embodiments, the method further includes the step of staining the cells. In some embodiments, the method further includes the step of treating the cells with a nuclear identifier prior to the performing a first analysis step, and the first analysis comprises analyzing the monolayer of cells to detect cells positive for the nuclear identifier. In some such embodiments wherein the nuclear identifier comprises SYBR Green the step of performing a first analysis includes the steps of illuminating the cells with a SYBR Green excitation light and detecting a SYBR Green emission light from the cells. In some such embodiments, the step of illuminating includes the steps of illuminating with a blue light and detecting comprises detecting a green light.

In some embodiments, the step of performing a first analysis on the monolayer of cells includes the step of detecting hemoglobin. In some such embodiments wherein the step of detecting hemoglobin includes the steps of illuminating (e.g. such as diffusing) the monolayer of cells with a hemoglobin absorbable light (such as, e.g. with a wavelength between 400 nm and 600 nm or between 400 nm to 450 nm) and detecting light from the monolayer of cells based on hemoglobin light absorption. In some such embodiments, detecting hemoglobin includes the steps of illuminating the monolayer of cells with a hemoglobin-absorbable light

In some embodiments including the step the detecting hemoglobin, the method further includes the step of detecting hemoglobin wherein a cell positive for a nuclear identifier and positive for hemoglobin comprises a candidate fetal cell.

In some embodiments, the step of performing a second analysis on the cell further includes the steps of performing an analysis on a control cell on the substrate and comparing a result of control cell analysis with a result of the fetal cell analysis.

In some embodiments, the step of performing a second analysis includes the step of performing at least one of immunocytochemistry and in situ hybridization, the method further including the step of fixing the cell on the substrate prior to the performing a second analysis step. In some such embodiments wherein performing a second analysis includes performing immunocytochemistry, the method further includes the steps of using an anti-hemoglobin antibody and obtaining an immunocytochemical signal to thereby confirm candidate fetal cell identity. In some other such embodiments wherein performing a second analysis includes performing immunocytochemistry, the method further includes using at least one of an anti-fetal hemoglobin antibody and an anti-embryonic hemoglobin antibody and obtaining an immunocytochemical signal to thereby confirm candidate fetal cell identity. In some other embodiments, the method further includes the step of thresholding a value of the immunochemical signal value wherein a signal value above a first threshold value, a signal value below a second threshold value, or both confirms a candidate fetal cell identity. In yet some other such embodiments, wherein the step of performing a second analysis on the cell includes the steps of analyzing cellular DNA by in situ hybridization and obtaining a signal based on the DNA; and comparing the signal with a reference characteristic (such as, e.g. a reference DNA characteristic based on DNA from a maternal cell or based on DNA from more than one individual) and thereby determining a genetic characteristic of the potential fetal cell. In some embodiments the step of determining includes the step determining least one of a chromosome number (such as, e.g. of at least one of chromosome 13, chromosome 18, chromosome 21, an X chromosome, and a Y chromosome), a DNA duplication, and a DNA deletion.

In some embodiments, the method further includes the steps of removing a first potential fetal cell from the substrate after the performing step; obtaining DNA from the cell; and amplifying cell DNA (such as, e.g. using polymerase chain reaction (PCR) for such as less than 30 cycles). In some embodiments, amplifying includes the step of performing at least one of quantitative polymerase chain reaction and digital polymerase chain reaction. In some such embodiments, the method further includes the steps of analyzing the DNA using a fetal identifier wherein a positive signal is indicative of a fetal cell. In some such embodiments, wherein the performing step further includes the steps of distinguishing a plurality of potential fetal cells (such as, e.g. from the same monolayer or from a plurality of monolayers), the amplifying step further includes the steps of amplifying DNA for less than 30 cycles from each of potential fetal cells of the plurality of cells to obtain a plurality of DNA samples, the analyzing step further includes the step of analyzing the plurality of DNA samples using the fetal identifier to obtain a plurality of fetal cells each having a positive signal, the method further including the step of pooling the DNA from plurality of fetal cells each having a positive signal to obtain pooled, amplified DNA. Some embodiments further include the step of performing a second amplification on the DNA.

In some embodiments, the step of analyzing includes the step of sequencing at least a portion of the DNA or performing array Comparative Genomic Hybridization (array CGH) and comparing a signal with a reference characteristic, such as from a maternal DNA signal or from a reference DNA including DNA representing more than one individual.

In some embodiments, the step of analyzing includes the step of detecting at least one of a chromosome number (chromosome 13, chromosome 18, chromosome 21, an X chromosome and a Y chromosome), a DNA duplication, a DNA deletion, and a single nucleotide polymorphism (SNP).

Some embodiments further include the steps of enriching the maternal blood sample to thereby generate a sample enriched in maternal white blood cells and at least one fetal cell having at least a 5000:1 to 1:10 ratio of red blood cells to white blood cells in the enriched maternal blood sample; self-assembling a live-cell monolayer wherein at least a portion of the assembled cells is removably attached to the surface or not attached to the surface; treating the cells with a live-cell nuclear stain; optically detecting stained cells; illuminating (e.g. with a diffuser) the cells with a light between 400 nm and 600 nm and detecting hemoglobin wherein cells positive for a nuclear identifier and positive for hemoglobin comprise potential fetal nucleated cells; placing the potential fetal cell on a substrate after the selectively removing step and performing a second analysis on the cell to determine if the potential fetal cell is a candidate fetal cell; repeating at least the selectively removing and placing steps to thereby obtain a plurality of candidate fetal cells; and performing a second analysis comprises performing immunocytochemistry using an anti-fetal hemoglobin and an anti-embryonic antibody on the plurality of potential fetal cells wherein a positive signal above a threshold indicates that a potential fetal cell is a candidate fetal cell. In some embodiments, the method further includes the steps of: obtaining DNA from each of the candidate fetal cells; amplifying DNA from each of the candidate fetal cells; analyzing the DNA pool by at least one of DNA sequencing and array Comparative Genomic Hybridization to obtain a fetal DNA profile; and comparing the fetal DNA profile with a reference characteristic to thereby determine a genetic characteristic of the fetal cells wherein the genetic characteristic comprises at least one of aneuploidy of chromosome 13, 18, 21, X, and Y.

Another aspect of the invention provides a method of separating a candidate fetal cell from a maternal blood sample. In some embodiments, the method includes the steps of: placing a sample of maternal blood enriched for fetal cells on a surface wherein the sample comprises maternal nucleated cells and a fetal cell; removably attaching at least a first portion of the cells in the sample to the surface; performing an analysis on the cells to identify a candidate fetal nucleated cell; and removing the candidate fetal nucleated cell from the surface to thereby isolate the cell from the remainder of the cells.

In some embodiments, the step of removably attaching includes the step of loosely adhering the cells to the surface such that a cell may be removed intact from the surface using a micropipette and a partial vacuum.

In some embodiments, the method further includes the step of adding a material (e.g. such as bovine serum albumin (BSA), such as between 0% and 0.05% (w/v) BSA) to at least one of the surface and the sample wherein the material is configured to reduce cell adhesion to the surface.

In some embodiments, the method further includes the step of maintaining a second portion of cells close to the surface.

In some embodiments, the method includes the step of placing a sample of maternal blood on the surface includes the step of placing viable cells on the surface, the method further including the step of maintaining cell viability at least through the removably attaching step.

In some embodiments, the method includes the step of removing a candidate fetal nucleated cell includes the step of subjecting the cell to a partial vacuum to thereby remove the candidate fetal nucleated cell.

In some embodiments, the step of removing a candidate fetal nucleated cell includes the step of gathering the cell in a micropipette having an inner diameter less than 30 μm. In some embodiments, the step of removing a candidate fetal nucleated cell includes the step of gathering the cell in a micropipette coated with bovine serum albumin (BSA), such as, for example, less than about 1% (w/v) bovine serum albumin.

In some embodiments, the providing step includes providing a transparent, rigid surface. In some embodiments, providing includes providing a functionalized surface on a transparent backing. In some embodiments, providing includes providing a PEGylated (polyethylene glycol functionalized) surface.

Yet another aspect of the invention provides a method of identifying a candidate cell. In some embodiments, the method includes the steps of providing a maternal blood sample comprising hemoglobin containing cells; assaying a first fetal hemoglobin level in a first cell; providing a minimum threshold fetal hemoglobin level; and identifying the first cell as a candidate maternal cell if it has a hemoglobin level below the minimum threshold hemoglobin level and as a candidate fetal cell if it has a hemoglobin level at or above the minimum threshold hemoglobin level.

In some embodiments, the method includes the step of applying an algorithm after the providing step.

In some embodiments, the method includes the step of forming a monolayer from the maternal blood sample prior to the assaying step, wherein assaying a first fetal hemoglobin level comprises assaying a first fetal hemoglobin level in a cell in the monolayer. In some embodiments, the method includes the step of performing immunocytochemistry to thereby detect the fetal hemoglobin level in the first cell. In some embodiments, the method includes the steps of assaying a second fetal hemoglobin level in a second cell, wherein the second cell includes a candidate fetal cell, and providing a threshold fetal hemoglobin level includes the step of providing a threshold fetal hemoglobin level lower than the fetal cell hemoglobin level. In some embodiments, the method further includes the steps of assaying a fetal hemoglobin level in each of a plurality of candidate fetal cells, wherein providing a minimum threshold fetal hemoglobin level includes the step of providing a level based on the fetal hemoglobin levels in the plurality of candidate fetal cells.

In some embodiments, the step of assaying further includes the step of enumerating signals using a processor and applying an algorithm to thereby identify the candidate cell.

In some embodiments, the method further includes the step of detecting a perimeter of the first cell wherein assaying comprises assaying a first fetal hemoglobin level encircled by the perimeter.

Yet another aspect of the invention provides a method for attaching a fetal cell to a porous membrane. In some embodiments, the method includes the steps of placing a solution configured to maintain fetal cell integrity on a porous membrane; placing a potential fetal cell in the solution on the membrane; and removing the solution and thereby adhering the potential fetal cell to the porous membrane.

In some embodiments the step of removing the solution includes the step of evaporating the solution. In some embodiments the placing step includes the step of placing the solution on a porous polyester membrane (such as, e.g. polyethylene terephthalate (PET) or polyethylene naphthlate (PEN)).

In some embodiments the step of placing a solution includes the step of placing a solution volume between about 50 fl and about 10 ul. In some embodiments wherein the cell is in the solution and the steps of placing a solution and placing a cell are performed simultaneously.

In some embodiments, the method includes repeating the placing a solution, placing a potential fetal cell, and the removing the solution steps to thereby attach a plurality of cells with the membrane wherein each cell is separated from any other cell on the membrane.

In some embodiments, the method includes the step of exposing the porous membrane to a controlled environment having less than 100% humidity (such as, e.g. less than 70% humidity) during at least a part of the removing step.

Yet another aspect of the invention provides a method for removing a cell. In some embodiments, the method includes the steps of providing a cell attached to a portion of a flexible surface; apposing at least the portion of the flexible surface to a resilient substrate; surrounding the cell with a hollow shaft; and applying a force between the hollow shaft and the flexible surface wherein the force is opposed by the resilient substrate, to thereby separate the portion of the flexible surface and the cell attached thereto from the rest of the flexible surface.

In some embodiments, the step of providing a cell includes the step of providing a cell separated from any other cells on the flexible surface. In some embodiments, the step of providing a cell includes the step of providing a cell attached to biaxially-oriented polyethylene terephthalate (Mylar).

In some embodiments, the method further includes the step of analyzing a characteristic (such as, e.g. performing at least one of a DNA analysis, an RNA analysis, a protein analysis, and an optical analysis) of the cell attached to the surface before the surrounding step to thereby to thereby determine to remove the cell. In some embodiments in which analyzing a characteristic includes performing a protein analysis, the method further includes performing immunocytochemistry. In some of these embodiments wherein the cell includes a fetal cell, performing immunocytochemistry includes analyzing at least one of fetal hemoglobin and embryonic hemoglobin.

In some embodiments, the method further includes the step of connecting the hollow shaft with a microscope turret prior to the surrounding step. In some embodiments, the method further includes the step of calibrating a location of the hollow shaft relative to the flexible membrane. In some embodiments, the method further includes the step of providing a signal indicating that the portion of the flexible surface has been separated from the rest of the flexible surface after the applying a force step. In some embodiments, the method further includes the step of applying a vacuum after the applying a force step to thereby hold the separated portion of the flexible surface and the cell attached thereto.

In some embodiments, the method further includes the step of placing the portion of the flexible surface and the cell attached thereto in a solution configured to perform at least one of detaching the cell from the membrane and extracting DNA from the cell.

In some embodiments, the method further includes the step of controlling the surrounding and applying steps with a microprocessor.

In some embodiments, the method further includes the steps of: repeating the providing, opposing, surrounding, and applying steps to obtain a plurality of cells; obtaining DNA from the plurality of cells; and amplifying the DNA using PCR to thereby obtain amplified DNA; and obtaining an average amplified DNA yield per cell of at least 0.35 μg when using less than 36 cycles of PCR.

Another aspect of the invention provides a cell separating device. In some embodiments, the device includes: a shaft comprising a first hollow end and a second end; and a turret housing connected with the shaft and configured to connect the second end with a microscope objective turret on a microscope wherein the shaft is configured to remove a portion of a flexible substrate when the turret housing is in place in the microscope objective turret and the flexible substrate is in place on a microscope stage on the microscope.

In some embodiments the hollow end includes a flattened end or a beveled end.

In some embodiments an inner diameter of the first hollow end is between about 200 um and about 400 um and/or an outer diameter of the first hollow end is between about 300 um and about 500 um and larger than the inner diameter. In some embodiments, the shaft includes a portion of a 25 gauge needle. In some embodiments, the shaft includes stainless steel.

Another aspect provides a kit for enriching for fetal nucleated cells. In some embodiments, the kit includes a filter configured to capture fetal nucleated red cells while allowing a substantial portion of non-nucleated red blood cells to flow through; and an instruction for use. In some embodiments, the kit includes an elution buffer. In some embodiments, the kit further includes a syringe pump configured to connect with the filter and control an elution rate of the filter.

Another aspect of the invention provides a cell separating system including a cell separating device including a shaft having a first hollow end and a second end, and a turret housing connected with the shaft and configured to connect the second end with a microscope objective turret on a microscope wherein the shaft is configured to remove a portion of a flexible substrate when the turret housing is in place in the microscope objective turret and the flexible substrate is in place on a microscope stage on the microscope; and the flexible substrate.

In some embodiments, the cell separating system further includes a feedback source configured to provide an indication that a portion of the flexible membrane has been removed by the cell separating device. In some of these embodiments, the feedback source includes at least one of a microphone, an amplifier, and a band-pass filter. In some embodiments, the system further includes a motorized stage controlled configured to respond to a feedback signal from the feedback source to thereby move the motorized stage to a second location. In some of these embodiments, the system further includes a microscope.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-2 show a process and system for enriching for fetal nucleated cells from a blood sample using a filter system.

FIG. 3 shows a cell smear tool creating a layer of cells on a surface.

FIG. 4 shows a system for automating the process using a device such as the one shown in FIG. 1.

FIG. 5 shows an automated system used to identify fetal nucleated cells.

FIG. 6 shows cell analysis results after enriching for fetal nucleated cells according to one aspect of the disclosure.

FIG. 7 shows a system for automating the process using a device such as the one shown in FIG. 1.

FIG. 8 shows a cell smear from a pregnant female.

FIG. 9 shows a method for detecting and distinguishing fetal cells from maternal cells according to the disclosure.

FIG. 10 (1-2) describes methods for obtaining fetal cells.

FIG. 11 describes hemoglobin expression at different stages.

FIG. 12 shows expression of fetal hemoglobin during various genetic states or conditions.

FIG. 13 shows a method for analyzing cells according to one aspect of the invention.

FIG. 14 shows the performance of a prototype system to characterize fetal cells.

FIGS. 15A-B show results of a short tandem repeat (STR) analysis to identify fetal cells.

FIGS. 16A-C show results of an array Comparative Genomic Hybridization analysis to identify fetal characteristics from pooled fetal cells according to the disclosure.

FIGS. 17A-B show a monolayer of cells illuminated using 420 nm light to identify hemoglobin (FIG. 17A) and analyzed by fluorescence for nuclei (FIG. 17B).

FIGS. 18A-C show a detail view of a portion of the monolayer shown in FIGS. 17A-B using 420 nm light hemoglobin (FIG. 18A), fluorescence for nuclei (FIG. 18B), and both (FIG. 18C).

FIG. 19 shows a cell being removed from a flexible membrane according to one aspect of the disclosure.

FIGS. 20A-B shows cells on a flexible membrane analyzed for the presence of nuclei and hemoglobin.

FIG. 21 shows a portion of a monolayer analyzed using 420 nm light for hemoglobin, fluorescence for nuclei, and both together.

FIG. 22 shows results of various monolayer analyses using an automated analysis system according to the disclosure.

FIGS. 23A-B show two images of the same monolayer identifying a fetal nucleated red blood cell.

FIG. 24A shows a device and system for removing a cell from a substrate.

FIGS. 24B-C show cross-sections of the device from FIG. 24A for removing a cell from a substrate.

FIG. 25A shows components that may be part of a system for removing a cell from a substrate.

FIG. 25B shows the components from FIG. 25A assembled for receiving a cell.

FIG. 26 shows a system for removing a cell from a substrate.

### DETAILED DESCRIPTION OF THE INVENTION

In general, a fetal status analysis needs to provide accurate, reliable, and reproducible data in order to draw meaningful conclusions. Obtaining questionable or downright erroneous results can be unacceptable. Such results may lead to parental distress, false conclusions, and dangerous and/or unnecessary testing or procedures. Some fetal cell analyses (e.g. including some described herein) require high quality DNA in order to provide the necessary high quality data with which to draw meaningful conclusions. DNA quality, especially when the DNA is in a cell that is outside its natural environment or present in a very small quantity or present at a low concentration, can readily be compromised or destroyed by heat, nucleases, chemical treatments, and other factors. Passage of time, excessive manipulation, and exposure to fixatives and stains can all contribute to diminished DNA quality. Thus, fewer steps and less manipulation are generally preferred in order to obtain high quality DNA from a sample.

Although described in detail for detection of fetal blood cells, the methods, materials, kits and systems may also useful for obtaining and analyzing other types of cells, such as circulating tumor cells, stem cells, red blood cells, or other cells (e.g. rare cells) from a blood sample. The methods, materials, kits and systems may also be useful for obtaining and analyzing other types of cells, such as dispersed cells from a tissue sample.

Numerous challenges need to be overcome in order to readily provide accurate genetic information about a developing fetus. One challenge lies in preventing harm to the developing fetus. Another challenge lies in minimizing harm to the pregnant female. Another challenge lies in having sufficient quantities of fetal material for performing an analysis. Another challenge lies in consistently obtaining fetal material that meets or exceeds all minimum quality requirements for a highly accurate genetic analysis. Another challenge lies in providing reliable fetal genetic information from pregnant females having any of a wide variety of health conditions. Another challenge lies in overcoming technical limitations of a particular genetic analysis method in order to provide highly accurate results from a very small sample. Yet another challenge lies in providing genetic results in a cost-effective way. The best way to address any one of these challenges may have negative or disastrous consequences for any of the other goals. For example, a process that maintains fetal cell morphology such as by fixation may cause irreparable damage to the DNA for use in molecular biology testing. In short, determining fetal genetic status is still a challenge.

Part I

Enrichment Methods

Fetal cells and fetal DNA circulate in the pregnant female's bloodstream. One approach to providing genetic information about a developing fetus is to analyze fetal cells from the pregnant female's bloodstream. This approach brings with it an additional set of challenges. The number of fetal cells in the bloodstream is very small compared with the number of maternal cells. And, in spite of decades of work since the initial suggestions and scientific reports showing that maternal blood contains fetal cells, a reliable method to provide genetic information about a developing fetus from fetal cells in a pregnant female's bloodstream has not materialized. One challenge is in obtaining a sufficient number of cells to provide sufficient material for genetic analysis. To provide information about a developing fetus, an enrichment method may be used to reduce the number of cells that need to be probed for analysis in order to minimize cost and maximize throughput. One challenge is in retaining a cell of interest with sufficient morphology to enable subsequent identification. Furthermore, since nucleated red blood cells (nRBCs) as a class of cells can be fetal or maternal, another challenge is centered around developing a marker that provides high (e.g. 100%, though less may be acceptable in some circumstances) specificity for fetal cells over maternal cells. Another challenge is in retaining sufficient quantities of sufficiently high quality fetal cell material (e.g. protein, nucleic acid) to allow fetal cell analysis to be performed. Again the best way of achieving one of these goals may have negative or even disastrous consequences on any of these goals or any of the general goals listed above.

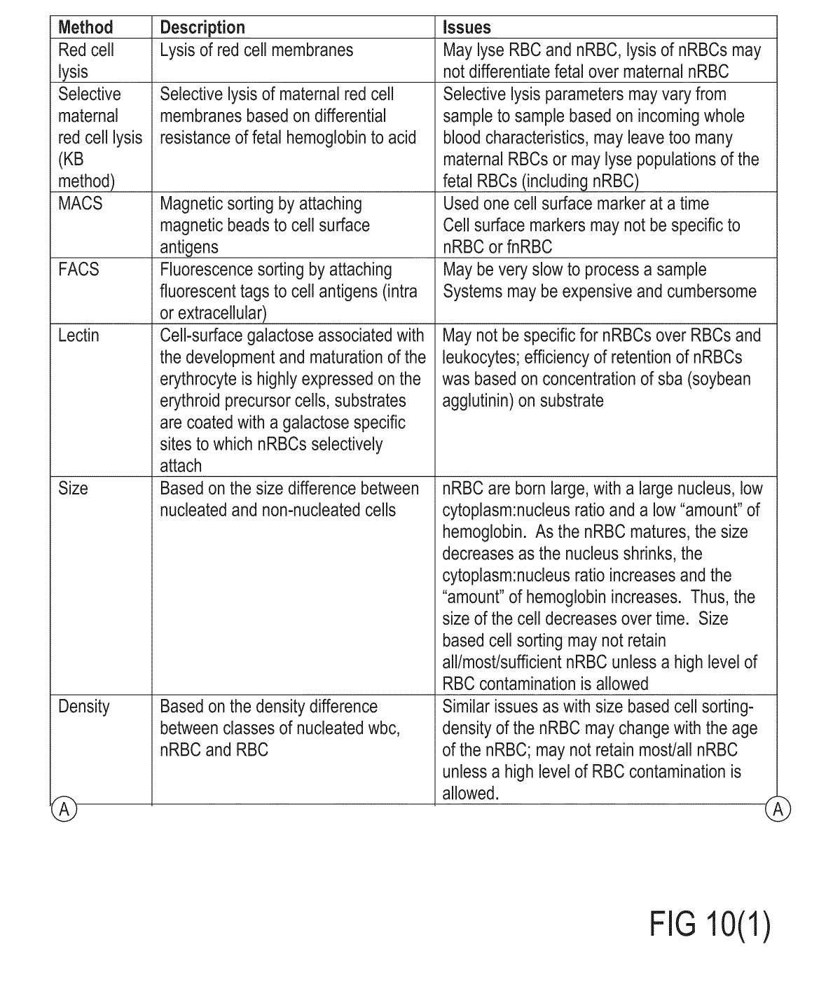

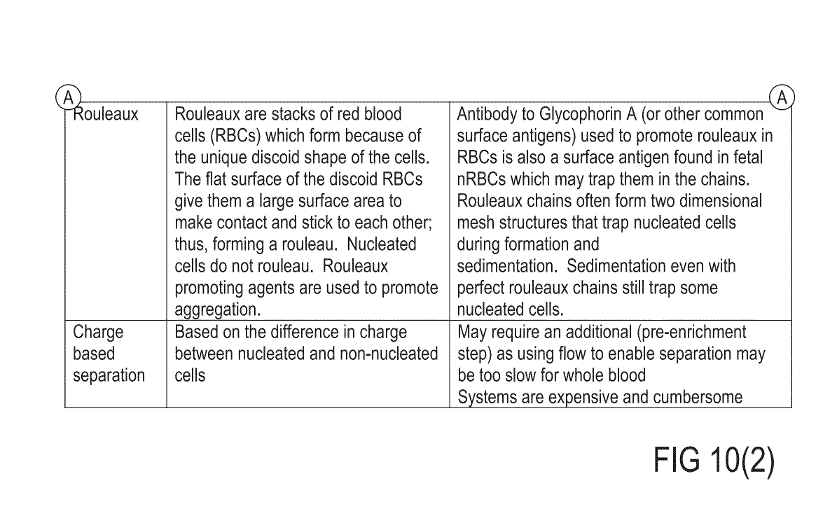

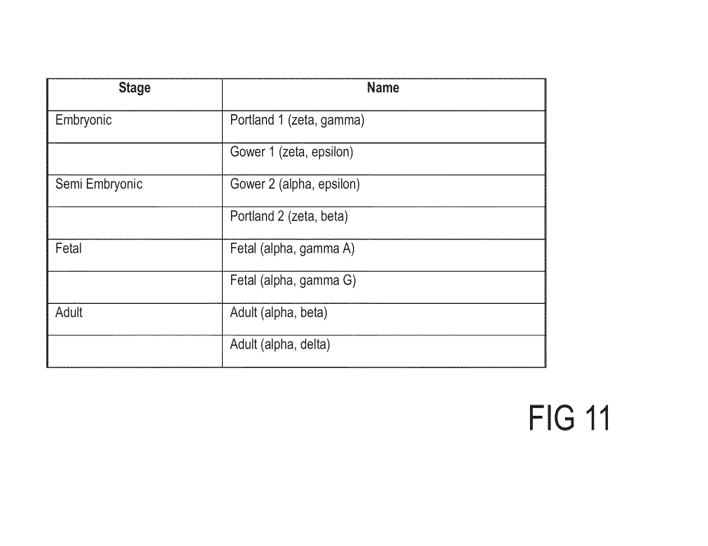

Historically, many enrichment methods, either alone or in combination, have been employed to solve this problem with varying degrees of success. FIG. 10 (1-2) summarizes some approaches and issues that have been encountered with these approaches As discussed above, the methods available for enrichment have not led to a system for efficient cell enrichment (or separation) and recovery.

Enrichment

Maternal blood contains both nucleated (i.e., containing genetic material) and non-nucleated cells of both fetal and maternal origin. In order to focus attention on the cells of most interest, a first step in fetal genetic testing may therefore be to concentrate nucleated cells within the sample. One prior approach is described in US Patent Appl. Publ. No. 2010/0159506. Since red blood cells are denser than white blood cells, a preliminary separation of red blood cells is obtained by a single density gradient to separate mononuclear cells, including nucleated red blood cells, from a whole blood sample. The sample is then applied to a slide in a monolayer, stained, and analyzed.

An additional method has been developed to obtain cells by employing a simple gravity filtration method. The method is based on leukodepletion technology and retains nucleated (e.g. essentially all nucleated) cells; no secondary enrichment is required. While not limiting the method to a specific mechanism of action, cell separation is thought to occur as a function of charge. The process is fast (may take about 15 minutes to separate nucleated cells from non-nucleated cells) and is very inexpensive.

Most leukocyte depletion filters in use today can be described as “Depth Filters”. In a depth filter, various types of fibers, such as polyester and polypropylene, are woven into layers to create a “torturous path” for the blood cells to pass through. The average “pore size” of the woven fibers is quite large—on the order of 40 microns or more—to avoid trapping cells by mechanical properties such as size. Instead, the trapping mechanism appears to be primarily that of “Adhesion” in which negatively charged leukocytes are attached or attracted to the fibers by Van der Waals and electrostatic forces. A fiber may be selected for its natural positive charge or can be coated to create very specific charge profiles and to ensure good hydrophilicity. An overall large pore size combined with the dense woven layers and charged fiber surfaces create a filter in which blood can pass through rapidly yet the individual cells follow a slower torturous path where they can adhere to the fibers.

In some embodiments, a leukocyte depletion filter was utilized, and it was determined that Nucleated Red Blood Cells (nRBCs) and more specifically Fetal Nucleated Red Blood Cells (fnRBCs) possess negative charge that is greater than the majority of common Red Blood Cells (RBCs) yet less than that of most leukocytes. The nRBCs and fnRBCs are retained in the leukodepletion filter, along with a small portion of the RBC population (in which the charge characteristics overlap) and, of course, all the leukocytes.

After the filtration process is complete and a sufficient number (e.g. many, most or all) of the sample's leukocytes, nRBCs, fnRBCs have been trapped (along with a population of RBCs), an “Elution” technique is used to recover the cells that have been trapped in the filter. A solution of charge neutralizing fluid (Dextran) can be reversed flushed through the filter to detach the cells from the positively charged fibers and into a collection container (e.g. tube or bag). In some embodiments, reverse flow tubing and a suitable valve structure may be coupled to the filter to enable the retrieval of the cells of interest.

Once enrichment is complete, in some embodiments the next step is to identify cells of interest. In some embodiments, fnRBCs are identified with 100% specificity. The process uses a combination of antibodies directed against embryonic and fetal hemoglobin for positive confirmation and can use in conjunction with a maternal red cell identifier as negative confirmation. Below is a discussion surrounding the theory of the marker combination.

Fetal Cell Identification

Red blood cells make up the bulk of circulating blood cells in the human. A normal blood cell has a life cycle as follows:

1. Proerythroblast: Large cells, 15-20 micron in diameter, large nucleus with 2-3 nucleolus, cytoplasm very less and basophilic with perinuclear halo and sometimes forms ‘ear-shaped” bulges, nuclear chromatin is non-homogeneous.

2. Early normoblast: Slightly smaller cells, 12-17 micron in diameter, reduction of nuclear size, disappearance of nucleolus, cytoplasm is moderately basophilic with high protein and RNA content, condensation of chromosomes occur.

3. Intermediate normoblast: Diameter of cells is 12-15 micron, nuclear size is further reduced having a “cart-wheel” appearance, chromosomes are further condensed, cell division stops totally, cytoplasm is polychromatophillic, appearance of hemoglobin takes place.

4. Late normoblast: Cells are reduced in size with 8-12 micron diameter, “ink-spot” nucleus and finally disappears; increase of hemoglobin amount, cytoplasm or pyknotic becomes acidophilic.

5. Reticulocyte: Size of cells are 8 micron in diameter, formation of highly branched pattern by remains of mitochondria, ER, ribosomal RNA—hence named as reticulocyte.

6. Erythrocyte: Mature RBC with 7.2 micron, 1.7 m thick in diameter, biconcave shape, and absence of nucleus, high hemoglobin content makes the cells typically pink red in color.

Each hemoglobin molecule has a lifespan of approximately 100-120 days, equal to the red blood cell it is contained in. Each mature red blood cell normally contains 26-34 picograms of Hb at a concentration of 310-360 g/L.

Hemoglobin is present in all enucleated (without nuclei) red blood cells (erythrocytes) and nucleated red blood cells (erythroblasts). The protein molecule transports oxygen from the lungs to other parts of the body tissue and transports carbon dioxide from tissues to the lungs. Hemoglobin can be expressed at high levels and represent up to 95% of the solid constituents of red blood cells.

Hemoglobin molecule is composed of 4 polypeptide subunits or chains and 4 heme groups.

Hemoglobin Structure

The globin fraction of hemoglobin includes 2 pairs of polypeptide chains; one pair of alpha-like chains and one pair of non-alpha chains. The chains are designated by Greek letters (alpha-α, beta-β, delta-δ, epsilon-ε, and gamma-γ).

Various chains of hemoglobin are expressed at different stages of gestation.

Gene Expression of Hemoglobin

The predominant hemoglobin present in embryonic red blood cells is embryonic hemoglobin (epsilon chain) and the predominant hemoglobin present in fetal red blood cells post the embryonic stage is fetal hemoglobin (gamma chain). Thus, one part of the process may employ a combination of antibodies against epsilon globin and gamma globin. A potential source of interference comes from F cells, defined as a cell containing gamma globin, present in a percentage of the red cells in normal adults and in a percentage of red cells in adults affected with certain conditions that promote the expression of gamma globin.

Normal adult: F cells are believed to be present in all adults at low concentrations. The amount of gamma globin on a per cell basis is low (e.g. <15%), whereas the amount of gamma globin on a per cell basis for a fetal cell will be high (e.g. >90%). This allows for a thresholding algorithm to discriminate fnRBCs from maternal F cells.

Affected adult: F cells are also present in adults affected with conditions that promote the expression of gamma globin. In each case, either the level of expressed gamma hemoglobin is low and thresholding can be applied, or the level of expression is high and prevalent in a large concentration of maternal cells allowing for exclusion of these samples based on the number of positive expressing cells.

Although the developed algorithms are specific to fetal cells over maternal RBCs and maternal F cells, the process may also, or alternatively, employ a third surface marker, carbonic anhydrase, which is specific to maternal red cells to confirm that the target cell is fetal and not maternal.

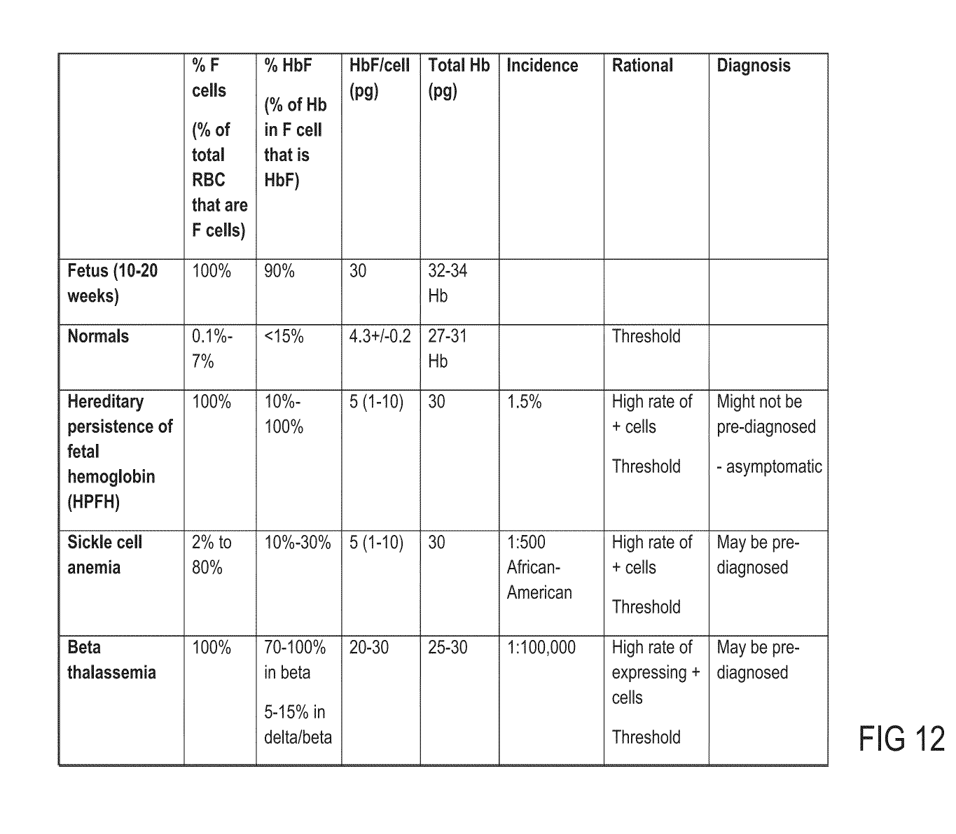

Maternal F Cell Interference (See FIG. 12)

In general, thresholding has been performed to effectively subtract (non-specific) background staining for discrimination of cells that are positive or negative for antibody staining. Thresholding, however has not been previously employed to eliminate non-specific staining due to contamination or specific staining where the concentration of the target is at a different level.

The process employs the following techniques to identify fetal cells in the presence of background, non-specific staining and specific staining of maternal F cells:

Background: “Subtract” background staining present on maternal nucleated cells by simple thresholding.

Non-specific staining: Identify non-specific staining morphologically by determining if it is a cell, if it has a nucleus and if it is a mononuclear cell with a cytoplasm; identify non-specific staining optically by determining if it is debris by examining the object under 420 nm and determining if it is “stained” in all fluorescent channels in the same pattern.

Specific staining of maternal F cells: Effectively subtract maternal F cells that express a specific, but low level of staining by simple thresholding; employ exclusion criteria for samples where a large number of highly expressing F cells are present in a “normal” patient as being “affected” and not analyzable.

Although the technical challenge has centered around enrichment and identification of fetal nucleated red blood cells, turning these solutions into a commercially viable process also poses technical challenges given that the enrichment process yields a large number of cells that need to be individually analyzed. Thus, a system needs to be built around handling and evaluating a large number of cells. One solution includes: sample with fetal material, enrichment (remove non-nucleated RBCs, retain nucleated cells), stabilization (maintain morphology and enable monolayer), monolayer (ensure access to nucleated cells by minimizing overlap), fix (maintain viability of DNA for genetic analysis and allow for antibody staining, antibody staining (boost dynamic range of signal to allow thresholding), scanning (provide uniform illumination and consistent focus), image analysis (apply algorithms to threshold signal), fetal cell identification (human review to confirm fetal cells), fetal cell extraction (capture, such as commercially available laser capture of identified cells), DNA extraction, fragmentation, and amplification (such as using a commercially available kit), purification (such as using a commercially available kit), genetic analysis (such as usually a commercially available system). Steps may be eliminated or changed or new steps may be added without departing from the invention. Steps may include:

1. 20 ml of maternal blood may be drawn into ACDa vacutainer tubes. EDTA may be included or added. A sample may be processed within 24 hours of draw. A sample may be stored above room temperature, below room temperature, or at room temperature. Storage above room temperature may lead to degradation. In one example, the sample is maintained at about room temperature (around 24° C.; at a range of about 20° C.-25° C.

Storage and transport: Storage of uncentrifuged blood beyond 24 hours may result in undesirable changes in the sample. There may be significant changes in analytes, for example, due to movement of water into the cells resulting in hemoconcentration, leakage of intracellular constituents, and coagulation. Fetal cells are fragile and thus may be more susceptible to these deleterious changes.

2. In one embodiment, a preliminary separation of red blood cells by simple filtration separates nucleated cells, including nucleated red blood cells, from a whole blood sample. A filter device used for filtration may retain the cells by any means (e.g. size, charge or ionic interactions, non-ionic interactions). In one embodiment, the filter retains nucleated cells by charge. A fluid may be applied to remove cells from the filter. A fluid may serve to neutralize or buffer a charge on the surface of the cells (and/or on the filter) in order to elute cells from the filter.

Dextran and sugars: Dextran is a neutral branched polysaccharide made from glucose monomers. The dextran may serve the purpose of releasing the leucocytes and other blood cells from the filter membrane. The cells may be originally collected via charge affinity between the cell membranes and the charged filter surface. The dextran, although uncharged, may serve to reduce this interaction between cells and the charged surface and solubilize the cells. The dextran may also reduce the extent of crystallization by salts and disaccharides. Additional sugars trehalose and maltose may be used in the subsequent wash step. Trehalose and maltose are disaccharides known in the literature as agents for improving membrane stability during biopreservationl. The disaccharides are believed to have a favorable effect on the phase behavior of the phospholipid bilayer due to hydrogen boding. Although these beneficial effects have been observed during different conditions such as cryopreservation, it is believed that the disaccharides have a beneficial effect on the preservation of cell membrane integrity during centrifugation. In some embodiments, a phosphate buffered saline (PBS) solution may be used as the elution fluid.

3. A stabilizers may be added to preserve the integrity and morphology of the cells. Examples of stabilizers and other materials that may be added alone or in combination include, but are not limited to:

RNA Stable: The cell pellet may be transferred to a centrifuge tube that contains RNA stable (Biomatricia). The product includes stabilizers, a very small quantity of phenol red dye and EDTA to stabilize samples with nucleic acid at room temperature. The materials in this formulation are water soluble, and might not remain after processing the slide.

Sodium Fluoride: Sodium fluoride in PBS may be used in the resuspension solution of the pellet. Fluoride salts are good at stabilizing proteins against denaturation. This salt may have a dual action on blood samples. It may be used in conjunction with oxalic acid to prevent coagulation or clotting of blood and secondly it may inhibit glycolytic enzymes responsible for the breakdown of glucose in the blood.

Numatrix: Numatrix {QC Sciences) may facilitate cell spreading. Numatrix contains approximately 2% by weight of a high molecular weight polyethylene oxide polymer and 5% ethanol. Polyethylene oxide is a very biocompatible, nonionic, highly soluble polyether. In the cell pellet the polymer is believed to coat and encapsulate the cells with minimal disruption of the hydrophilicity of the cell surface. By incorporating the polymer into the suspension prior to spreading, the suspension may be given a more consistent viscosity from sample to sample. Viscosity may be an important parameter that correlates with monolayer density and the deposition speed. In addition to yielding a more consistent viscosity, the polymer may make the resulting interaction between the depositing cells and the surface more lubricious. A more lubricious surface may help to get a more uniform monolayer and to reduce shear stress on cells depositing on the surface. The small quantity of ethanol is incorporated and may increase the rate of initial evaporation.

D×H Diluent with Trehalose and Maltose: A volume D×H solution containing maltose and trehalose may be added to the suspension. The disaccharides may be added as preservatives for the same beneficial reasons mentioned above because most of the sugars present before centrifugation are decanted from the cell pellet thus necessitating the augment. The D×H diluent (Beckman Coulter) contains sodium sulfate, imidizole, sodium chloride and tetracaine HCL, in decreasing order of relative quantities. Sodium sulfate and sodium chloride may be incorporated to make the solution a suitable isotonic diluent for blood cells. Imidizole is a heterocyclic base which is used in washes for histidine base binding assays to reduce nonspecific binding and is an antimicrobial agent. Tetracaine HCL is a compound that inhibits proteins from spontaneous release of calcium.

4. The sample may then be applied in a monolayer to a slide using a monolayer technology and subsequently air-dried. One goal is to maintain access to most or all retained cells without overlap.

One goal—5000:1 to a 1:10 RBC:WBC: A filter, sample treatment, or filter elution protocol may be chosen to optimize the number of fetal nucleated RBCs obtained by enrichment. In order to retain more or essentially all of the nucleated fetal red blood cells from a maternal blood sample, a relatively higher number of non-nucleated red blood cells may also be retained or a lesser number might be retained. Thus a ratio of RBC:WBC of 5000:1 to 10:1; 10:0 to 1:1, 1:1 to 1:10 may be chosen.

One Goal <2:1 RBC:WBC: The quality of a mechanically spread monolayer may be a function of, among other things, the rheology of the cellular mixture being spread. With one embodiment of the blood smearing process described herein a composition of approximately 2 erythrocytes to 1 leukocyte may be optimum for some applications. With higher erythrocyte ratios there may tend to be two-dimensional segregation of cells with larger free glass void areas in between. In addition, because there are approximately 120 million leucocytes in a sample, a higher erythrocyte ratio means that a substantially larger area and greater number of cells have to be scanned and analyzed. With too small of an erythrocyte concentration the incidence of bilayers and cell stacking may increase. The consistent size and shape of the erythrocytes and their stronger tendency to sediment may yield a better monolayer in some cases when they are mixed in an approximately 2:1 ratio with leucocytes.

In addition to rheological advantages the erythrocytes form a natural spacer between leucocytes. Search algorithms described herein and in copending patent application Ser. No. 13/046,543 filed Mar. 11, 2011, identify regions of nucleated cells. With erythrocytes serving as natural spacers, individual nucleated cells may be better differentiated and target fetal cells may be more easily isolatable from surrounding cells without nuclear contaminants from adjacent nucleated cells.

5. The cells may then be fixed using a non-crosslinking fixative in order to maintain the viability of the DNA for subsequent analysis, and frozen until antibody labeling. Streck (STF) is a non-crosslinking agent; unlike aldehyde fixatives (e.g. formaldehyde and glutaraldehyde), there is no crosslinking of proteins or nucleic acids, which might otherwise affect antigen availability and probe penetration. STF includes urea in an alcoholic solution with other reagents. Urea is a weak denaturing agent thought to destabilize structure rather than completely unwinding a protein. Alcoholic solutions may enhance penetration past the cell membrane and fix cells/tissue to slides. Alcohol is a weak fixative, and potentially may denature/destabilizing proteins and make them sticky. There are other agents in STF for buffering, osmotic stability, mordants, nuclei defining, and penetrating agents.

6. Antibody labeling may be conducted using a process optimized to label expected concentrations of hemoglobin (e.g. embryonic or gamma) or other cell markers throughout gestation. An amplification method may be used to boost the signal in order to reduce the subsequent scanning time.

7. A scanning system, designed to reduce scan time, may be employed to acquire images. The system may include a high intensity, narrow band, multi-colored excitation source, which allows for reduced exposure time and independent balancing of channels for multiplexing of wavelengths. A high resolution, large format camera may be used to acquire images at low magnification, further reducing scan time.

8. An algorithm(s), including those described herein, may then be employed. An algorithm may provide a list of possible targets for further analysis or subsequent human review. Methods may be employed to evaluate signal over background and maternal F cell interference where the expression of gamma globin is low.

9. An operator may evaluate hits and determine if the cells are fnRBCs based on developed acceptance criteria. A list of X, Y coordinates of accepted cells may be stored for subsequent processing.

10. A commercial laser capture system or other system may be used to extract the cells from the substrate.

11. Commercially available kits may be used for DNA extraction, fragmentation & amplification.

12. An off the shelf purification column may be used to purify the amplified product.

13. The material may be taken through an array CGH process.

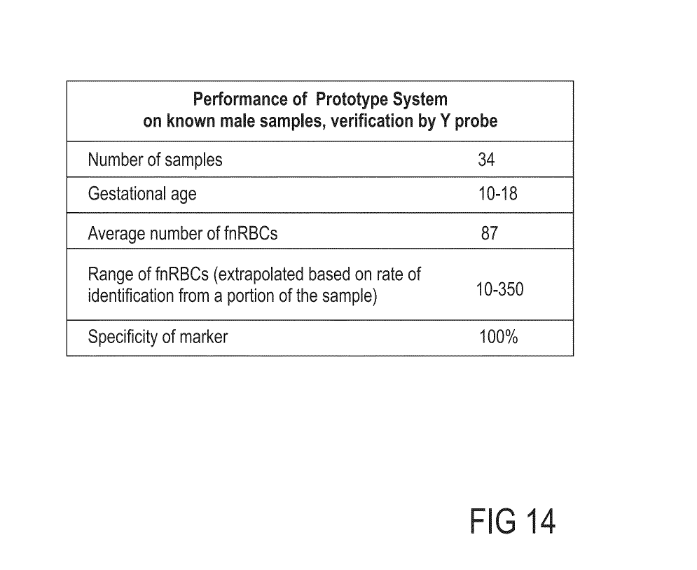

A prototype system has been used to collect feasibility data on the sensitivity and specificity of the fetal marker. Results using the system are shown in FIG. 14. The system has been modified to include X, Y FISH post antibody labeling in order to confirm fetal-ness of the identified cells. The Y probe on known male samples is used as a gold standard.

Flow Cytometry

The front end enrichment technology and method for fetal cell identification are enabling for the identification and isolation of fnRBCs in maternal circulation. The question of “is there another way to do this” has plagued researchers, both academic and commercial, for 30 years. Flow cytometry is a powerful tool for the selection of cells, including rare cells. Some current systems may be too slow for performing a primary enrichment, as the amount of time required to process 100 billion cells from 20 ml of whole blood would be extraordinarily long—approximately 2 months. However, the process may be sped up, fewer cells may be analyzed in some cases, or it may be combined with any of the methods described herein. An alternative method for cell identification and isolation, after an initial enrichment (e.g. after filtration) may be flow cytometry.

However cell identification (using antibody labeling as an example, but the process is applicable to any identification method) on a cell smear may have the following advantages over flow cytometry.

1. Antibody labeling for flow may require a hard fixative and then permeabilization which may be detrimental to array CGH quality.

2. Antibody labeling for flow may be conducted in solution. A solution based process might require additional (e.g. 6 additional) wash steps, each with a centrifugation step, which is harsh and likely results in cell loss, morphological damage and aggregation.

3. Specificity of flow sorting may not be 100% and potential for contamination is high, which may then require a visualization step to confirm the cell type and the staining. Thus, flow sorted cells may still require application to a substrate and subsequent extraction or application to a microwell technology. Microwell technologies may be subject to yield issues.

4. Historical data has shown poor yield and reproducibility.

Part II

Leukocyte depletion filters can be used to filter blood products from whole blood. Information about leukocyte depletion filters may be found in the following: U.S. Pat. No. 5,676,849; U.S. Pat. No. 5,662,813; U.S. Pat. No. 6,544,751; U.S. Pat. No. 4,923,620; U.S. Pat. No. 4,925,572; Comparison of Five Different Filters for Removal of Leukocytes from Red Cell Concentrates; VOX Sang 1992/62:76-81; Recovery of Human Leukocytes from a Leukodepletion Filter; Chang et al; J. Transfusion 1992/32 (85); Recovery of Functional Human Lymphocytes from Leukotrap Filters; Longley et al.; J. Immunological Methods; 1999/121:33-38; Biotechniques 31:464-466 (2001); S. Ebner et al.; J. Immunological Methods; 2001(252):93-104. See e.g. See U.S. Pub No. 2011/0311960, U.S. Pub. No. 2012/0021508, and WO2011/14055. In one aspect of the invention, such filters can be used to separate nucleated blood cells from non-nucleated blood cells.

According to one embodiment, a first step of the method is filtration. The incoming blood sample is filtered (e.g., within 24 hours of draw, preferably within 12 hours of draw) using a leukocyte depletion filter. A suitable leukocyte depletion filter is the PureCell™ Select System from Pall Corporation.

Custom chemistry may be used for cell suspension during the filtration process. The chemistry may include trehalose, maltose, dextran, pseudoephedrine, and salts of fluoride, phosphate and/or sulfate. The chemistry may address issues related to the degradation of cells during processing using the manufacturer's recommended process; improve the cell morphology in the final sample and may enable the unique DAPI+420 nm imaging process; reduce cell clumping and sphering which improves the ability to spread the cells evenly in a large area monolayer; stabilize the cells and provide more margin in terms of the time between the start of the filtration process and the creation of the monolayer; and reduce cell destruction and release of DNA into the suspension. The chemistry may also be used to increase the allowable time between blood draw and processing which will expand the population of candidate patients by opening the test to doctors in areas that are not near metropolitan centers.

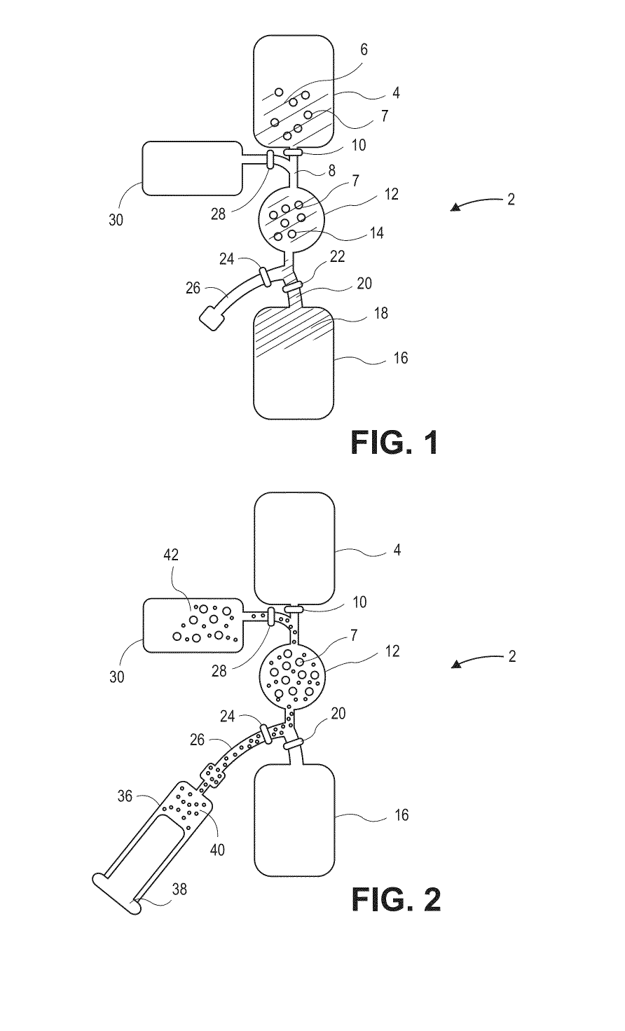

FIGS. 1-2 show a process and device for enriching for fetal nucleated cells from a blood sample using a filter system according to one embodiment of the invention. A maternal blood sample 6 containing various components of red blood including nucleated maternal and fetal nucleated cells 7 such as fetal nucleated red blood cells, is contained in bag 4. Valve 28 connecting tubing 8 to collection bag 30 and valve 24 connecting tubing 20 to tubing for elution 26 are closed. Valve 10 connecting tubing 8 to filter 12 is opened and blood sample 6 is passed through filter 12. Cells for collection 14, including nucleated fetal cells 7, remain behind on filter 12, while unwanted blood products 18, including some mature reticulocytes, pass through filter 12 and are collected in waste collection bag 16.

After unwanted blood products 18 (e.g., most non-nucleated cells) have passed through the filter, the nucleated cell population remaining on the filter contains the cells of interest, nRBC's, as a subpopulation. Other cells, including some non-nucleated red blood cells, may also remain on the filter. Having some non-nucleated red blood cells present during the later steps may improve the smear quality and/or enable a more uniform monolayer. As shown in FIG. 2, valves 10 and 22 are closed. A syringe 36 containing elution solution 40 is attached to tubing 26. Valves 24 and 28 are opened. A syringe 36 containing elution solution 40 is attached to tubing 26. Valves 24 and 28 are opened. Elution fluid 40 is forced backward through the filter and causes the nucleated cells 7 to be released from the filter. An elution fluid such as one recommended by the manufacturer of the filter may be used. Alternatively, one aspect of the invention includes use of an elution fluid containing one or more of sodium or potassium fluoride (e.g., 0.01 to 100 mg/mL), pseudoephedrine (e.g., 0.1 to 100 mg/L), EDTA (e.g., 1 to 10 mM), ACD-A (Acid Citrate Dextrose (e.g., 0.01 to 20%) trehalose (e.g., 0.01 to 10 gm/L), maltose (e.g., 0.01 to 10 gm/L), dextran (e.g., 0.01 to 10.0%, 10,000-150,000 daltons; e.g. 70,000 daltons in one example), Pluronic® F-68 (e.g., 0.001 to 10 mg/mL), sodium or potassium sulfate (e.g., 1 to 100 mM), or mixtures or salts of sodium or potassium hydrogen phosphate or sodium or potassium dihydrogen phosphate (e.g., 1 to 100 mM; pH in the range of about 5 to about 9) and Tetronic® 1107 (1 to 100 mM). In one embodiment, the elution solution includes about 25 mg maltose and about 75 mg trehalose in about 25 mL phosphate buffered solution (PBS). Elution fluid 40 and cells 7 are collected in collection bag 30 to yield an enriched fetal nucleated cell population 42. These released cells and the elution fluid are collected (e.g. into a vial).

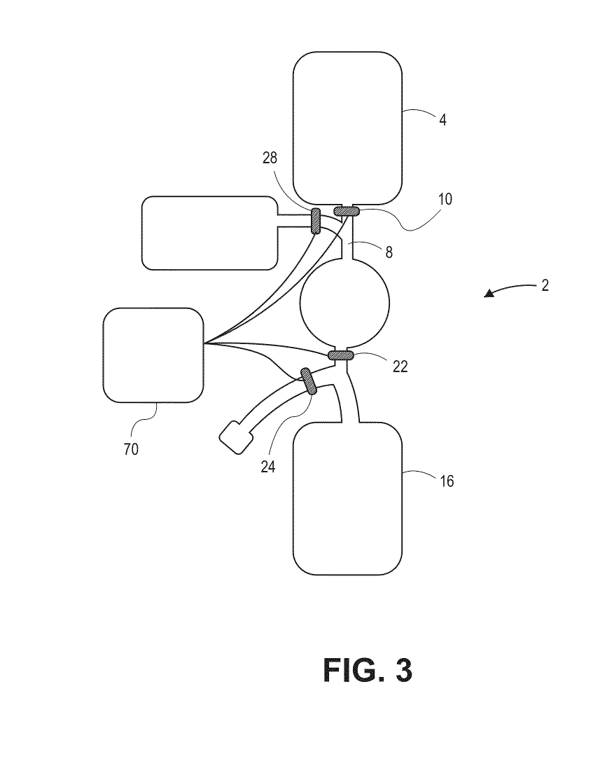

Machinery may be provided to automate the filtration and cell release process. The machinery may control any or all of the flow rate, the pressure and the amount of fluid that moves through the filter system. FIG. 3 shows controller 70 according to an embodiment of the invention configured to control flow rates of sample, waste, elution fluid, and/or elution fluid containing fetal nucleated cells through valves 10, 28, 22, and/or 24. A flow rate equal to 0.2-50 ml/second may be used. The volume of fluid that moves through the filter system may be from 5 mL to 500 mL. Controlling flow rates can have a significant impact on the cell population that is recovered: If the flow rate during filtration is too high, cells of interest may be driven deeply into the filter making them harder to recover. Also, if the flow rate is too high during filtration, backflush, and/or elution, cells may be damaged by hydrodynamic forces. If the flow rate is too low on the release backflush, cells of interest may not be recovered; they may remain trapped by the filter. Machinery for automation of filtration and cell release process may include a computer controlled valve and pump system whereby the fluid flow paths through the filtration device can be controlled by a series of valves and manifolds. One type of valve particularly well suited is the pinch tube valve since it can control fluid flow without coming in contact with the fluids. This is advantageous for avoiding cross-contamination between samples and making for a fully disposable filtration system where expensive components such as valves do not need to be discarded or cleaned. The flow through the filtration device can be accomplished by a combination of gravity and mechanical pump systems such that both rate of flow and flow velocity profiles can be set and controlled by computer hardware and software.

The machinery may also improve the repeatability of the process and reduce cycle time. For example, the automation may enable high speed processing while maintaining standardization of sample handling. Also, the filtration process requires control of valves, volumes, and flow rates during the process. The automation will minimize operator/technique dependent variability. In some embodiments, the machinery to control volume, pressure, and/or flow rate may include a syringe pump. For example, the pump may include a specific volume of fluid and may be emptied within a specific time. As a specific example, 30 mL may be emptied into the filtration system in 3 seconds. The pump may be a manual pump or a computer and/or motor operated pump.

The next step may be concentration by centrifugation. The cells may be stained and counted (e.g., using the optical density of the suspension as a measure of cell count) before centrifugation. Alternatively, or additionally, cell count can be determined by hemocytometry before centrifugation with or without staining. Alternatively, or additionally, cells may be stained after centrifugation. After the cells have been collected in a centrifuge tube (having, e.g., total volume of approximately 50 ml dilute cell suspension), the tube is spun in a centrifuge (e.g., for 15 minutes at 500×g at room temperature) to concentrate the cells into a pellet at the bottom of the tube. The resulting pellet volume is approximately 100 μl to 300 μl. It is possible to stain the cells before centrifugation as well as count the number of cells in the pre-centrifuge suspension.

Measuring the cell count in the pre-centrifuge suspension, for example by using the optical density of the suspension or through the use of a hemocytometer, allows the automation of the removal of supernatant and the automation of the addition of stabilizers and stain (e.g., 0.01-20% ACD-A, 0.01 to 10% of 10 K-400 K daltons dextran, 1-10 mM EDTA, 0.01 to 10 g/L maltose, 0.1 to 100 mg/mL pseudoephedrine, 0.01 to 10 g/L Trehalose, 0.01 to 100 mg/mL sodium or potassium fluoride, 1 to 100 mM sodium or potassium phosphate, and/or 1 to 100 mM sodium or potassium sulfate. In one embodiment, stabilization fluid may contain sodium sulfate (less than about 20 grams per L), sodium chloride (less than about 2 grams per L), imidazole (less than about 3 grams per L), sodium fluoride (about 2 grams per L), and pseudoephedrine (less than about 0.1 grams per L). In some cases, measuring the cell count after centrifugation may lead to large errors in cell counts because the volume is so small and the optical density is very high (opaque). Alternatively and/or additionally the pH or ionic strength of the suspension can be adjusted after enrichment, but before centrifugation. Alternatively or additionally ionic or non ionic surfactants can be added after enrichment and before centrifugation.

Automating the volume of stain and stabilizer that is added to the suspension prior to centrifugation allows the stain and stabilizer to reach and affect all cells in a more homogeneous manner. Adding stain and stabilizer after centrifugation in some cases may lead to non-homogeneous staining and stabilization because the cells have already started to clump and the large solid to liquid ratio in the pellet may inhibit the even distribution of the additives.

Adding a nuclear stain prior to the creation of the monolayer reduces the number of processing steps required to enable scanning. In some cases, staining after the monolayer has been created requires a fixation step that adds complexity, handling, and cost to the sample processing and may modify the cell morphology, reducing the effectiveness of the automated digital microscopy.

Automating the amount of supernatant that is removed after centrifugation may improve the repeatability of the final cell suspension that is used to create the monolayer. Standardizing the solid to liquid ratio of the monolayer suspension generates more uniform distribution of cells on each substrate and also improves the variability between individual smears on slides. Maximizing the cell distribution uniformity enables the extrapolation of one slide's results to all slides in a single patient set. This ability to accurately extrapolate results means that a smaller number of slides must be put through the imaging system and reduces the necessary cycle time per patient. In addition, tight control of the suspension density minimizes or eliminates the need for a trial and error approach to creating a monolayer with an appropriate cell density for automated digital microscopy.

In some embodiments, after concentration in the centrifuge, the cells are resuspended. Using the cell count as a guide for the final required liquid volume, unwanted suspension fluid is removed from the centrifuge vial until only the desired total volume remains in the vial. The remaining supernatant and cells are then gently mixed (e.g., by agitation or by repeated aspiration and dispense cycles using a pipette). Automated removal of the supernatant based on the pre-centrifugation cell count enables the creation of a monolayer that is optimized for automated digital microscopy and rare cell identification. The cells may be resuspended in any composition suitable for preparing a layer of cells on a substrate. The cells may be resuspended and/or further prepared in phosphate buffered saline (PBS), sodium fluoride, Coulter® diluent (D×H), alcohol (e.g. isopropanol) and/or alcoholic agarose (e.g. QC Sciences NuMatrix™). Alternatively and/or additionally the composition of the resuspended pellet can be controlled by adjusting the solid to liquid ratio, by adding polymers such as polyethylene oxide to change viscosity, by changing buffers to change the pH, by adding adhesion promoters or adhesion retarders to affect surface affinity of cells, by adding chelating agents to sequester divalent cations, and changing ionic strength.

Next, a monolayer is created. A monolayer may be created using any method or any devices. A monolayer may be created using a smear tool or may be self-assembling, or both. A cell sample for making a monolayer may contain non-viable cells (e.g. fixed cells) or viable cells, or both. A monolayer may be created on any surface. In some embodiments, the viability of cells may be maintained through the step of self-assembling a monolayer.

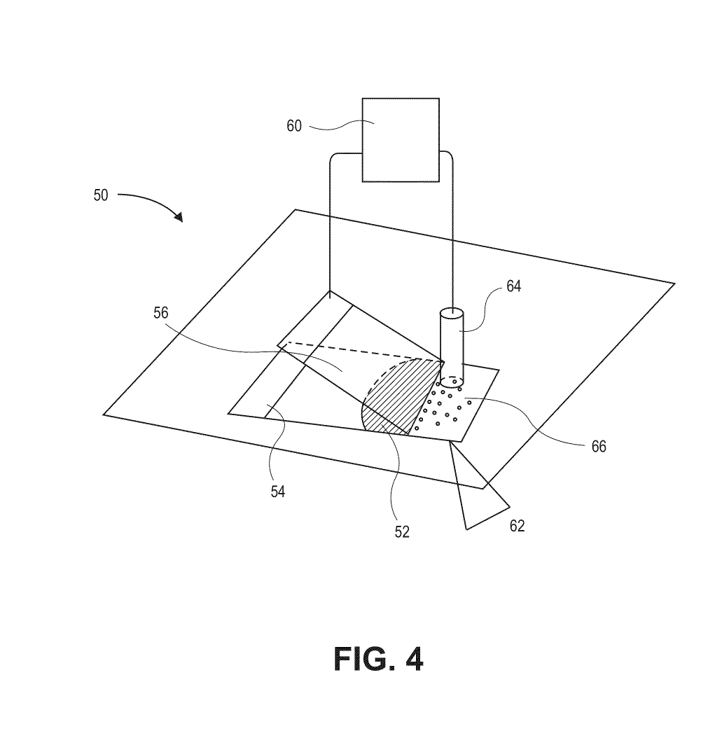

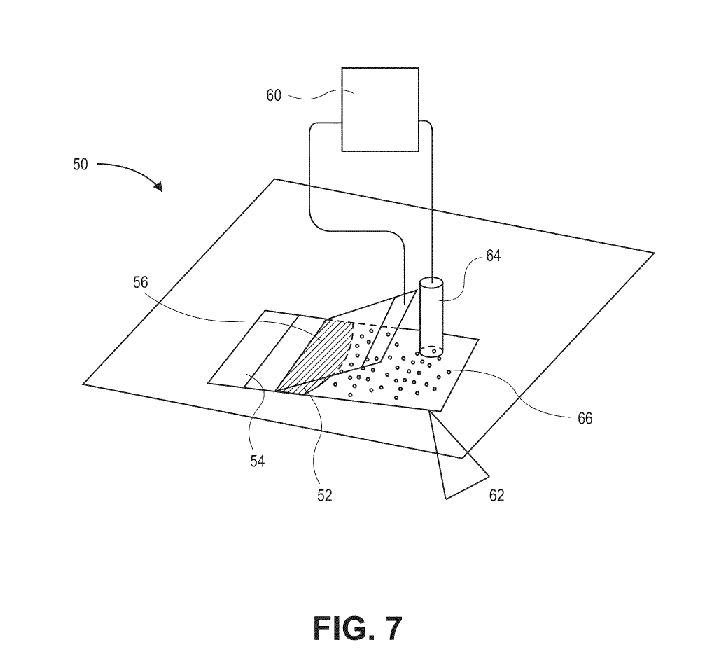

In one embodiment for making a monolayer, a resuspended pellet (or filter eluate) is then dispensed onto a substrate. Any substrate that allows a monolayer to be formed and allows analyses to be performed thereon may be used, such as glass, poly-n-isopropyl acrylamide (NIPAM), permanox, polycarbonate, polyethylene terephthalate (PETG), polystyrene, and thermanox. A surface may be a functionalized surface on a different substrate, such as a PEGylated (polyethylene glycol) coated or covalently attached surface on a glass substrate. In some embodiments, a substrate for making a monolayer is a transparent, rigid surface. A transparent surface allows light to pass, which is useful for performing an optical analysis in some embodiments, such as, for example, shining or detecting light from below the substrate. In one example, clear glass of standard microscope slide thickness is used. Using an automated smear tool, this droplet is spread evenly across the substrate surface in a manner that minimizes clustering and overlap of cells while maximizing the number of cells per unit area. FIGS. 4 and 7 show versions of a cell smear tool 50 according to some embodiments of the invention. A sample of enriched fetal nucleated cells 52 is placed on surface 54. Cell smear blade 56 is moved by cell smear tool controller 60 across sample of enriched fetal nucleated cells 52 to create a layer of cells 66 on surface 54.

Prior automated smearing tools merely recreated the human motion that has historically been used to create blood smears by moving only with a single action linear speed motion in one axis (e.g., along the long Y axis of the slide). In one aspect of the invention, the automated smear tool moves in both X and Y while making essentially a monolayer smear. The motion patterns of a stage (e.g., an XY motorized stage) controlled by the computer software are unique. In some embodiments, the motion goes across the short X axis of the slide. The use of the short X axis motion can be helpful in creating a suspension along the smear slide before actual forward motion of the smear begins. In some embodiments, the software runs open loop (without monitoring cell density).

In other embodiments, cell density is monitored. In FIG. 4 light 62 illuminates layer of cells 66 and images are captured by detector 64 and used to determine cell density. In one embodiment, feedback from detector 64 is used to guide controller 60 to change parameters of cell smear blade 56 to control the density of cells 66 on surface 54. The smear density could be monitored, e.g., by using blue light (which will be absorbed by the hemoglobin) or red light (which will measure all cells). Higher attenuation of the light indicates a denser cell layer; lower attenuation indicates a less dense cell layer. In some embodiments, the system is configured to vary parameters such as angle of smear and/or speed of smear in real time to assure that the uniformity of the monolayer is maintained.

The automated smear tool varies its speed during the monolayer smearing process to control the density of cells that are applied per unit area to the substrate. The software speed may vary, for example, from 0.1 mm/sec to 500 mm/sec. The software may have a starting speed and ending speed for the smear tool which are the same, or they may differ from each other. In the case where the starting speed and ending speeds differ, the smear tool may change speed automatically in real time during the smear process. The rate of change may be linear or exponential. It could also vary sinusoidally or be varied using any other continuous function during the smear. The automated smear tool optimizes the initial droplet pickup and spread more or less normal to the main direction of smear. This may improve the overall uniformity of the smear normal to the main direction of smear.

The automated smear tool may move in a zigzag pattern during the smearing process to improve the homogeneity of the suspended cell population and the homogeneity of the cell population distributions per unit area. The automated smear tool can create smears in any size, such as 1″×2″ or 5″×5″ (125 mm×125 mm).

The smear tool may have any motion that aids in distributing the cells. The smear tool may move in both the X and Y directions during both the pickup of the initial cell droplet and during the actual smearing process itself. The motion(s) of the smear tool may be circular, zigzag, forward, backward, side to side with no forward or backward motion, diagonal, serpentine (move in +X, move in +Y, move in −X, move in +Y, repeat). These motions are useful for two reasons: they make the height of the meniscus even across the face of the smearing tool and improve the uniformity of the cell density in the direction normal to the main axis of the smear. Second, the starting and stopping motion helps to move cells up into suspension and improve the homogeneity of the cell population's distribution within the meniscus behind the smearing tool.

The automated smear tool may be controlled by a GUI that allows the user to set parameters such as smear speed, velocity profile, cross motion Y axis suspension parameters and motion distance. The parameters may be set up and stored based on an initial test smear.

The smear tool may have notched edges or polished edges, or it may be uncharged or have varying surface charge. The smear tools and/or smear slides may be made of glass or material other than glass.

In some embodiments, a monolayer may be partially or wholly self-assembling. A sample of maternal blood, which may have been concentrated, diluted, enriched, stained, and/or otherwise treated such as using any of the methods and/or materials described herein (above), may be placed on a substrate and may move or spread to thereby self-assembling a monolayer. The cells of a sample (and of a subsequent monolayer) may be viable or non-viable or may be mixed (viable and non-viable). Cells may self-assemble into a monolayer based on any principle. For example, a cell may move (e.g. crawl across a substrate, crawl past another cell, may move by diffusion, may move by convection currents, may settle, etc.). A substrate may be kept relatively immobile, or may be moved during monolayer self-assembly. A substrate may be rocked, swirled, or tilted relative to the cells during monolayer self-assembly in order to aid monolayer formation. A cell may be moved relative to the substrate, such as by moving a solution or drawing a solution across the substrate. In some embodiments, the substrate may be moved after the initial substrate formation, such that if any cells are stacked within the monolayer (for example, an undesirable stack of cells may include a white blood cell on top of a red blood cell), a top cell might be knocked off of an underlying cell. As described below, once a cell of interest has been identified, the substrate may be moved while visualizing the cell of interest to confirm that the cell of interest is not in fact one cell on top of another mimicking the characteristics of a cell of interest.

A self-assembling monolayer may form in any length of time (e.g. in less than 5 minutes, less than 10 minutes, less than 20 minutes, less than 30 minutes, less than an hour, less than two hours, or less than 5 hours). A self-assembling monolayer may form quickly or slowly, depending upon the assembly conditions, including, but not limited to whether a self-assembly temperature is at room temperature (68°), above room temperature, or below room temperature, the nature of the cell suspension or material (e.g. aqueous, gel, oil, etc.), and the nature of the substrate.

One aspect of the invention provides a method of placing a sample of maternal blood enriched for fetal cells on a surface wherein the sample contains maternal nucleated cells and a fetal nucleated cell; removably attaching at least a first portion of the cells in the sample to the surface; performing an analysis on the cells to identify a cell of interest; and removing a candidate fetal nucleated cell from the surface to thereby isolate the cell from the remainder of the cells.

A cell in a monolayer may be attached to the surface, removably attached to the surface, or may be close to the surface. A cell may be settled close to a surface. A cell settled close to the surface may be maintained close to the surface for e.g. at least 5 minutes, at least 15 minutes, at least 30 minutes, at least 45 minutes, at least an hour, or for less than 1 hour, less than 2 hours, less than 3 hours, or less than hours. A cell may be tethered to the surface. A cell in a monolayer may be loosely tethered (loosely adhered or loosely attached) or may be not attached to the surface. A removably attached or removably tethered cell may be readily removed using a pipette (micropipette) and a partial vacuum while maintaining the cell integrity. A non-removably tethered cell may be removed using any means, however a non-removably tethered cell may be partially or wholly lysed prior to or during removal. For example, a tethered cell may be removed using an external stimuli (such as temperature change with a NIPAM substrate) or an enzyme treatment (e.g. trypsin). Removably attaching cells includes loosely adhering the cells to the surface such that a cell may be removed intact from the surface using a micropipette and a partial vacuum (e.g. while maintaining cell integrity). In some embodiments, it is desirable to have at least a portion of the cells removably tethered to the surface. If none of the cells are removably tethered to the surface, the cells may be able to move within the monolayer. It may be difficult to scan and identify cells of interest as described below while the cells are moving. If a portion of the cells are removably tethered to the surface, those cells may function to corral or otherwise prevent the movement of cells that are not tethered to the surface.

A cell monolayer may have any density of cells. Cells may be spaced such that they are in contact or close contact with other cells or may be substantially separated from any other cells. In some embodiments, a monolayer may have a preferred density of cells. For example, too few cells may allow an average cell mobility to increase. For example, if there are fewer cells within the monolayer, they have more room to move around. If the cells are moving within a monolayer, it may be difficult to scan them and identify cells of interest as described below. Alternatively, if there are too many cells, the cells may be too crowded and may be more inclined to clump together and/or stack on one another. This may lead to false positives during the scanning and identifying steps described below. For example, in some embodiments, one cell on top of another (e.g. a white blood cell on top of a red blood cell) may mimic the characteristics of a cell of interest, thus creating a false positive. Furthermore, in some embodiments, with an increase in the number of cells within a monolayer, the longer the scanning and identifying steps will take. Increased time can have negative effects on cell viability and accuracy of the scanning and identifying steps. A sample of cells or a substrate may be treated in order to control monolayer formation and/or adhesion. If a cell adheres too tightly to a surface, it may be difficult to remove. A cell may be damaged and it may be difficult to subsequently analyze cell morphology or it may lose its DNA and/or DNA may be damaged. In one embodiment, a method of separating a candidate fetal cell from a maternal blood sample may include adding a material wherein the material is configured to reduce cell adhesion to the surface. A sample of cells may be treated with a material that reduces adhesion to a surface or increases adhesion to a surface, such as, for example bovine serum albumin (BSA). Any amount of BSA may be added that allows cells to form a self-assembling monolayer; and to attach, removably attach, and/or settle cells close to the surface. Between 0% and up to and including 0.05% BSA, between 0.05% and up to and including 0.1%, between 0.1% and up to and including 0.5%, between 0.5% and up to and including 1.0%, between 1.0% and up to and including 2%, or more than 2% w/v BSA may be added. In one example, about 0.02% w/v BSA is added (e.g. 0.02 grams BSA/100 mls PBS).

Any material may be used so long as it improves monolayer formation, DNA quality, and/or cell retrieval and does not negatively impact the analysis as a whole. A sample or substrate may be treated, such as with a cell adhesion molecule, collagen, other extracellular matrix components, fibronectin, other proteins, serum, serum albumin such as bovine serum albumin, and/or vitronectrin.

In a particular embodiment, a maternal blood sample is enriched for fetal nucleated red blood cells and contains white blood cells and maternal red blood cells. Viable cells in a solution containing 0.02% bovine serum albumin (BSA) are allowed to self-assemble on a substrate for about an hour to form a monolayer. Some cells are attached to the surface while other cells are close (not attached) to the surface. In some embodiments, a method includes maintaining a second portion of cells close to the surface.

In some embodiments, cell viability may be maintained from the time a blood sample is drawn from a pregnant female through monolayer formation. In some embodiments, placing a sample of maternal blood on the surface comprises placing viable cells on the surface, the method further comprises maintaining cell viability at least through the removably attaching step.

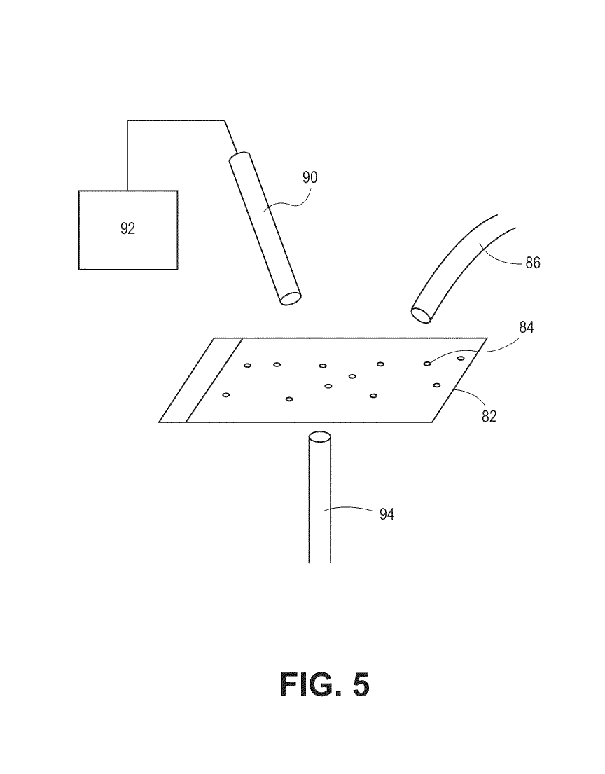

A cell monolayer may be fixed and imaged or may be imaged without fixing (or may be imaged before and after fixing). Imaging may be performed using an automated microscopy platform. In general any microscopy platform may be used. In one particular embodiment, a microscopy platform that has the capability to provide transmitted illumination (e.g., 380 nm-800 nm range) and coaxial illumination (e.g., in the 350 nm-364 nm range to excite a nuclear stain) may be used. FIG. 5 shows an automated microscope system that may be used to identify fetal nucleated cells according to one aspect of the invention. Light 86 or light 94 is delivered to surface 82 containing fetal nucleated cells 84 and other cells. Light from the cells on surface 82 is detected through microscope 90. Microscope 90 is moved relative to surface 82 by controller 92. Either the surface or the microscope may move. For example, if cells are in solution and/or are in or forming a monolayer and/or are loosely tethered or not tethered to a surface, a surface may remain stationary and a microscope may be moved across the surface to minimize the movement of the cells.

A light shaper may be utilized with a light source to improve image quality. A light shaper, such as a diffuser may diffuse a light so that it hits a cell from many angles. Using a diffuser may, for example, reduce a shadow and/or reduce an edge effect on a cell. A diffuser may be any material, such as ground glass, opal glass, “milk glass”, or any such as those described in U.S. Patent Publication 2007/0211460 to Ilya Ravkin, which is incorporated herein by reference in its entirety.

Any number of images using any source or sources of light may be collected. For example more than 100 images per substrate may be taken. In some embodiments, more than 500 images, more than 1000 images, or more than 2000 images may be taken. The XY locations, or points of view, on a substrate may be selected in such a way that the entire area of the substrate is imaged or just part of the substrate is imaged. The number of images taken and/or the size of each image taken may be determined based on an average diameter of a cell, or of a particular cell of interest. For example, the size of the point of view or XY location may be determined such that if a cell is not captured or is partially captured in a first image, it will be fully or at least partially captured in the adjacent image. In one embodiment, a pair of images is acquired for each XY location on the substrate. In some embodiments, the system and/or camera acquiring the images will perform an autofocus procedure prior to capturing the images. For example, the camera (or the microscope stage) will be moved in the Z direction (up/down) until the Z location where the sharpest image can be taken is identified. In some embodiments, the autofocus routine will be repeated prior to taking each image or prior to taking a pair of images in each XY location.

In one embodiment, each image acquired for each XY location may be acquired at two (or more than two) different wavelengths that are useful for detecting nuclei and detecting hemoglobin, respectively, in order to identify a candidate nucleated red blood cell (which, in this example, may be a fetal nucleated red blood cell or may be a maternal nucleated red blood cell). Any nuclear stain may be used. In some embodiments, DAPI or SYBR Green (an asymmetrical cyanine dye) may be used. For example, a first image may be detected using illumination at or around 420 nm, and a second image may be detected based on fluorescence detection. Fluorescence detection may be based on illumination around 358 nm for DAPI staining or 497 nm for SYBR Green staining and detection around 461 nm or 520 nm, respectively. For each location, an image pair may be used to determine a location in which the 420 nm light is absorbed which indicates the likely presence of hemoglobin. A fluorescent image may be used to determine the location of nuclear material in the field of view. Where the characteristics of shape and brightness of the nucleus match the known characteristics of a nRBC and hemoglobin is also present, a nRBC candidate has been found. This candidate may be added to a list of candidate cells. Hemoglobin absorbs a range of light wavelengths. Some embodiments include illuminating the monolayer of cells with a hemoglobin absorbable light and detecting light from the monolayer based on hemoglobin light absorption. A cell (in a monolayer) may be illuminated with any wavelength of light that hemoglobin absorbs, such as from 300 nm to 650 nm, from 400 nm to 600 nm, from 400 to 450 nm or from any of the peaks within these wavelengths (such as at or around (within 1, 2, 3, 4, 5, 6, 7, 8, 9, or 10 nm) 414 nm, at or around 542 nm, and at or around 576 nm) or around 560 nm, 580 nm, or 600 nm. A monolayer in which cells have been treated with SYBR green may be illuminated with any SYBR Green excitation light wavelength(s) and any SYBR Green emission light (e.g. any wavelength(s)) may be detected. In a particular example, a blue light (e.g. around 497 nm) is used for SYBR Green excitation and a green light (e.g. around 520 nm) is detected.

Note that red light in the range of 620 nm or 650 or higher may also (or instead) be used for analysis since this red light will not be absorbed by either nuclear material or by hemoglobin. A red image will thus (only) contain an actual physical structure of a cell: a cell edge, texture or background artifacts from the suspension medium, and non-cell particles on the slide. This true structure information may be used when determining the amount of hemoglobin signal that is coincident with nucleus locations and may be used to reduce false positives. (In some cases, it may not be possible using a 420 nm/fluorescent image pair only to separate a signal generated by structural edges from a signal generated due to 420 nm absorbance).

In some embodiments, a light (e.g. an OED light, or any light) may be shaped using a light shaper, such as a light diffuser. For example, a diffuser may be beneficial when analyzing a cell or cell monolayer for the presence of hemoglobin (e.g. hemoglobin absorption). In the absence of a diffuser, a non-hemoglobin containing cell (e.g. a maternal white blood cell) may diffuse or otherwise alter light and may erroneously appear as a hemoglobin containing cell (e.g. a nucleated red blood cell). For example, an edge of a cell may scatter light and thus appear as a dark ring, resembling a ring of hemoglobin. A diffuser may reduce or eliminate interfering white blood cell light anomalies. Conventionally, cells are placed between two panes of glass. The cells are typically placed with a medium having a refractive index similar to that of the glass, thus preventing the edges of the cells from scattering light. However, in the case of a live cell or self-assembling monolayer, this may not be desirable. Thus, alternative methods of eliminating scatter may be particularly useful. In some embodiments, alternative wavelengths that are close to a hemoglobin absorption peak may be used, while minimizing scatter. Some alternative wavelengths include 410 nm, 420 nm, 550 nm, 580 nm, 600 nm, etc. In some embodiments, the light scattering problem is solved with post processing and/or machine learning, thus enabling the system to distinguish between white blood cells and red blood cells illuminated with a standard LED light source.

An initial imaging may be done at a low magnification (e.g., 10×) to improve system speed and the highest scoring. nRBC candidates may be revisited at higher magnification to verify the status as true nRBCs. Additionally or instead, candidate nRBCs may be subject to human review

A high speed automated digital microscopy platform may be used to collect the images. Any set of image pairs (e.g., 420 nm/fluorescent) or triads (e.g., 420 nm, 620 nm, fluorescent) can be used to sort the cells into subpopulations of white cells, non-nucleated red cells, and nucleated red cells. A cell identification and subpopulation grouping algorithm does not interrogate cells that are known to be outside the population of interest.

The combination of the 420 nm and fluorescent imaging may be made possible due to sample preparation steps that preserve the morphology of the cells and maintain the hemoglobin intact through the creation of the monolayer. A unique algorithm can be used to determine the nRBC character of any cell. In one embodiment, the heart of the algorithm is the ability to determine the coincidence of 420 nm absorption and nuclear fluorescence (completely dark nRBCs) in addition to the adjacency of 420 nm absorption to nuclear fluorescence. The steps of the algorithm may include:

Flatten the illumination (make the image brightness even across the complete image to correct for dimness in the image corners which may arise due to optical system aberration).

Apply the image flattening to both fluorescent and 420 nm transmission images.

Segment the images to determine which pixels belong to foreground (nuclei and hemoglobin) and which pixels belong in the background. Image segmentation removes noise from the images and may reduce the number of pixels that must be processed in the remaining steps.

Enumerate (count and number sequentially) the individual nuclei in the fluorescent image.

Calculate their complexity and average brightness (#edge pixels2)/(# interior pixels) tends to 4π or 12.56 for perfectly round nuclei. nRBC nuclei tend to be round (low complexity) and bright.

For the subpopulation of the nuclei with the lowest complexity, determine the hemoglobin absorbance coincident to the nuclei and the hemoglobin absorbance around the perimeter of the nuclei. For example, the 50%, 25%, or 10% least complex are examined. (The higher nuclei do not need to be examined.)

Any parameters of the nuclei may be examined. In some embodiments, the brightness, complexity, and/or hemoglobin absorbance of the nuclei are measured. The cells that cluster in the 3-space region of high hemoglobin absorbance, low complexity, and high brightness have a high likelihood of being nRBCs and are graded as very likely candidates to be nRBCs.

In a similar manner, the white cells may be binned into their subpopulations (e.g., segmented neutrophils have high complexity and large whites), and every WBC that is put into a specific bin removes another cell from the nRBC candidate pool.

In some embodiments, both hemoglobin absorbance and nuclear fluorescence signals may be calculated relative to the average brightness of the foreground and/or the average or local brightness of the background. In this manner it is possible to make comparisons intra-site. (One pair of 420 nm/fluorescent images can be compared to another pair of 420 nm/fluorescent images.) If relative measures are not used then variations larger than a single field of view may impact the ability to correctly detect the cells of interest across the entire slide. Examples of large scale variations include: non-specific background, variations in fluorescent staining, variations in RBC hemoglobin lysing, and illumination changes (lamp warming up or failing).

In some cases, storage of the nRBC candidate XY locations may enable the use of less FISH or other reagents which reduces cost. The FISH or other reagents may be applied only to the sites where nRBCs have been located. After FISH is complete, these XY locations are used again to revisit the nRBCs and interrogate the final genetic testing result.

The image can then be analyzed. The use of the 420 nm and fluorescent image pairs (or a 420 nm, 630 nm, and fluorescent image trio) to find nRBC candidates depends on the following characteristics of nRBCs:
(1) nRBCs are associated with hemoglobin absorbance (dark pixels in the 420 nm image). Sometimes the hemoglobin absorbance area covers the same set of pixels as the nuclear fluorescence. In other cases the hemoglobin absorbance is adjacent to the fluorescent signature of the nuclear material.
(2) nRBC nuclei tend to be single nuclei and tend to be round (low complexity)
(3) nRBC nuclei tend to have a brighter fluorescent signal that other cell nuclei in the same image
(4) nRBC nuclei tend to be smaller than most other nuclei because they are in the process of condensing and being forced out of the cell (e.g., by apoptosis)

In some embodiments, once candidate cells are selected from the monolayer, a next step is the fixation step. The cells are treated with a non-crosslinking fixation in preparation for further analysis. Use of a non-crosslinking fixative or fixation in the presence of stabilizers which may serve to improve the quality of nucleic acid, protein (e.g. any antigenic sites), or other cell component, improve the quality of any amplified DNA, RNA or other component, or and improve any subsequent analyses on the nucleic acid, protein, or other cell components by FISH, PCR (e.g. dPCR, qPCR, smPCR) and/or array or microarray analysis (e.g. array comparative genomic hybridization. The fixation may maintain RBC morphology and hemoglobin signal for later relocation/revisit of the fetal nRBCs using the previously stored XY locations. Any fixation method that is non-crosslinking or causes only limited crosslinking may be used. Fixation may include EtOH (40 to 90%), glyoxal (0.1 to 25%), methanol (0.1 to 10%) and or isopropanol (0.1 to 10%) for (15 sec to 10 min) and may be preceded by a −20 degree centigrade methanol dip for 30 seconds to 2 days. Examples of suitable fixatives include, but are not limited to, Streck fixative (Streck, Inc., Omaha, Nebr.; such as those described in U.S. Pat. No. 5,460,797 to Ryan and U.S. Pat. No. 5,459,073 to Ryan), RCL2® alcohol based formalin-free fixative (Alphelys, Plaisir, France; such as those described in WO 2004/083369 and U.S. 2007/0072167 to Rochaix); and FineFIX (Milestone Microwave Laboratory System, Italy; such as those described in U.S. Pat. No. 6,042,874 to Visinoni et al.).

Stabilizers may include sodium or potassium fluoride (0.01 to 100 mg/mL), pseudoephedrine (0.1 TO 100.0 mg/L), EDTA (1 to 10 mM), ACD-A (0.01 to 10.0%) trehalose (0.01 to 10 gm/L), maltose (0.01 to 10.0 gm/L, dextran (0.01 to 3.0%, 100-500 MW), F-68 (0.001 to 10 mg/mL), sodium or potassium sulfate (1 to 100 mM), sodium or potassium phosphate (1 to 100 mM).

One suitable fixation process includes the following steps: Freeze substitution prefix in −20° C. MeOH for 10 minutes; and postfix treatment in EtOH, glyoxal, MeOH, and isopropanol. This process is beneficial in that it avoids the use of formaldehyde and glutaraldehyde. Waste disposal issues are eliminated, and DNA and RNA retrieval is made easier because no cross linking occurs.

In addition, human TSIX sequences may be used to definitively identify the cells as fetal female and not maternal. TSIX expression stops on both human X chromosome between 2 and 4 years of age. Adult females do not express the TSIX gene. Thus, including a probe for TSIX in the FISH process will allow a definitive determination of fetal female versus maternal status for all candidate nRBCs being interrogated. A FISH signal at the TSIX region of the nucleus will only be present if the cell is fetal. The presence of a Y chromosome determines if the nucleated red blood cell is from a male fetus.

It is also important to note that nuclei of the nRBCs are in the process of apoptosis and are being condensed in preparation for ejection from the cell. Highly condensed nuclei tend to have a lower efficiency from FISH treatment than do non-condensed nuclei. Two novel approaches to improving the FISH efficiency for condensed nuclei are:

(1) Perform FISH in a vacuum or under lower than atmospheric pressure; or

(2) Physically crush the cells and the nuclei prior to the application of the FISH probes by revisiting the XY locations of candidates and pressing on them in a controlled manner. The cells to be crushed or flattened would be the nRBCs identified by the DAPI/420 nm scan. On the same microscope platform that did the DAPI/420 nm scan a motorized nosepiece could rotate over the “crushing head”. This could be, for example, a spring loaded small diameter flat ended steel rod, and automatically lowered onto the slide at the location of the nRBC to be crushed. The crushing force applied may be controlled by the spring force constant in the crushing rod. The rod diameter may be small, for example, 100 microns in diameter so it would crush the target nRBC and the perimeter of cells around it. An absolute XY location accuracy may not be required. The small diameter also allows for very high crushing forces to be applied to the localized area.

The TSIX expression and the variation of TSIX expression versus human age is described in Species Differences in TSIX/Tsix Reveal the Roles of These Genes in X-Chromosome Inactivation; Migeon, Barbara R.; Lee, Catherine H.; Chowdhury, Ashis K.; Carpenter, Heather; doi:10.1086/341605 (volume 71 issue 2 pp. 286-293).

Crushing cells to improve access to the nuclear material is described in Cell Crushing: A Technique for Greatly Reducing Errors in Microspectrometry; Davies, H. G; Wilkins, M. H. F; Boddy, R. G. H. B.; Experimental Cell Research 6/(550-553); 1954.

Novel aspects of the invention include: The combination of TSIX with FISH for the definitive determination of fetal female/maternal status of cells; the use of vacuum to improve FISH results in fetal genetic testing; and the use of physical crushing of cells to improve access to the nuclear material.

Stored XY locations may be used to record genetic test results. The XY locations of the candidate nRBCs are used throughout the processing of the cells for genetic testing. It is possible to apply FISH probe to only those cells of interest, thus reducing the overall cost per test per patient. It is also possible to revisit the cells and physically remove them from the substrate for physical segregation away from the population of non-nRBCs, thus increasing the percentage of fetal DNA relative to maternal DNA. It is possible to revisit the XY locations of the nRBC candidates to interrogate the FISH results and fetal/maternal determination. It is also possible to revisit the XY locations of the nRBC candidates and physically decondense the nuclei.

The cells prepared by the methods described herein may be subject to antibody analysis. For example, several specific erythrocytic hemoglobin antibodies are available for the differential identification of fetal RBC's that occur in maternal peripheral blood (Zheng et al. 1999 Fetal cell identifiers: results of microscope slide-based immunocytochemical studies as a function of gestational age and abnormality. Am. J. Obstet. Gynecol. 180:1234-1239). Standard antibody staining techniques for fetal and embryonic hemoglobins may be performed on the nRBC's that are found in maternal blood by filtering samples, preparing monolayers on slides and locating nRBC's with an automated cell identification algorithm software. Adults do not express the embryonic hemoglobin, epsilon, while fetal RBC's may contain this embryonic hemoglobin up until the end of the first trimester (Mevron et al. 1999. Improved specificity of RBC detection in chorionic villus sample supernatant fluids using anti-zeta and anti-epsilon monoclonal antibodies. Feta. Diagn. Ther. 14:291-295). Antibodies against other embryonic (gamma, zeta) and fetal hemoglobins may be used with (or instead of) anti-epsilon to increase the specificity of identification of fetal nRBC's, but these antibodies may however also recognize zeta and/or fetal hemoglobin expression in adult sickle cell anemics and thallasemics. Fetal hemoglobin is expressed during the last two trimesters of pregnancy and shifts to beta-hemoglobin after birth. FIG. 12 shows expression levels of fetal hemoglobin (hemoglobin F or HbF) in a fetus, a normal adult, and in adults with various genetic conditions. Fetal hemoglobin staining of a blood sample from a pregnant female having one of these conditions or genetic statuses will identify both fetal and maternal cells as positive, specifically staining cells.

One object of the disclosure includes distinguishing a fetal cell from a cell from the pregnant female, including distinguishing a fetal cell from a cell from the pregnant female that expresses fetal hemoglobin.

One aspect of the invention a method of identifying a candidate maternal cell comprising: providing a maternal blood sample comprising hemoglobin containing cells; assaying a first fetal hemoglobin level in a first cell; providing a minimum threshold fetal hemoglobin level; and identifying the first cell as a candidate maternal cell if it has a hemoglobin level below the minimum threshold hemoglobin level and as a candidate cell of interest if it has a hemoglobin level at or above the minimum threshold hemoglobin level. Some embodiments include the step of applying an algorithm after the providing step. The method may also include forming a monolayer from the maternal blood sample prior to the assaying step, wherein assaying a first fetal hemoglobin level comprises assaying a first fetal hemoglobin level in a cell in the monolayer. A method may include performing immunocytochemistry to thereby detect the fetal hemoglobin level in the first cell.

In some embodiments, the method further includes assaying a second fetal hemoglobin level in a second cell, wherein the second cell comprises a candidate fetal cell, and providing a threshold fetal hemoglobin level comprises providing a threshold fetal hemoglobin level lower than the fetal cell hemoglobin level. In some embodiments, the method further includes assaying a fetal hemoglobin level in each of a plurality of candidate fetal cells, wherein providing a minimum threshold fetal hemoglobin level comprises providing a level based on the fetal hemoglobin levels in the plurality of candidate fetal cells. In some embodiments, the assaying further comprises enumerating signals using a processor and applying an algorithm to thereby identify the candidate cell. Some embodiments comprise detecting a first cell perimeter wherein assaying comprises assaying a first fetal hemoglobin level encircled by the perimeter.

Some methods according to the disclosure may include subtracting or removing part or all of a non-specific signal from a signal (e.g. from a pixel, superpixel, or image). Some methods may include using a first, non-specific signal to remove or reduce from consideration a strength or brightness of a second signal. A processor connected with a microscopy system may analyze a signal value obtained from an anti-hemoglobin-stained slide, apply a unique identification algorithm, and remove a non-specific signal from consideration. A non-specific signal may come from any source. A non-specific signal may be dimmer than a specific signal or may be brighter than a specific signal. A non-specific signal may come from staining in one or more cells due to any cause, including but not limited an interfering signal produced by a cell (e.g. auto-fluorescence or enzyme activity including but not limited to alkaline phosphatase activity), weak cross-reactivity or other stickiness of the antibody, non-uniform application of the antibody or any other reagents, non-uniform or incomplete washing or removal of unwanted reagent (e.g. antibody, detection reagent, or any other reagent), a handling or smear artifact, or background staining due to staining reagent remaining in a cell. A dimmer non-specific signal may come from a signal or staining that occurs on a slide or substrate, such as a signal produced by the slide or substrate. A non-specific signal may also come from a broken cell or other debris, cells or parts of cells that overlap.

A signal that is stronger than a specific signal may come from any unwanted source, including but not limited to any of the causes listed above. A method, according to the disclosure, may include applying an algorithm that removes from consideration a signal or image that is a minimum of 2×, 5×, 10×, or 50× brighter than a threshold.

The method may include removing a specific signal that is due to actual detection or staining of fetal or embryonic hemoglobin. A processor connected with a microscopy system may remove a specific signal or a specific image from consideration.

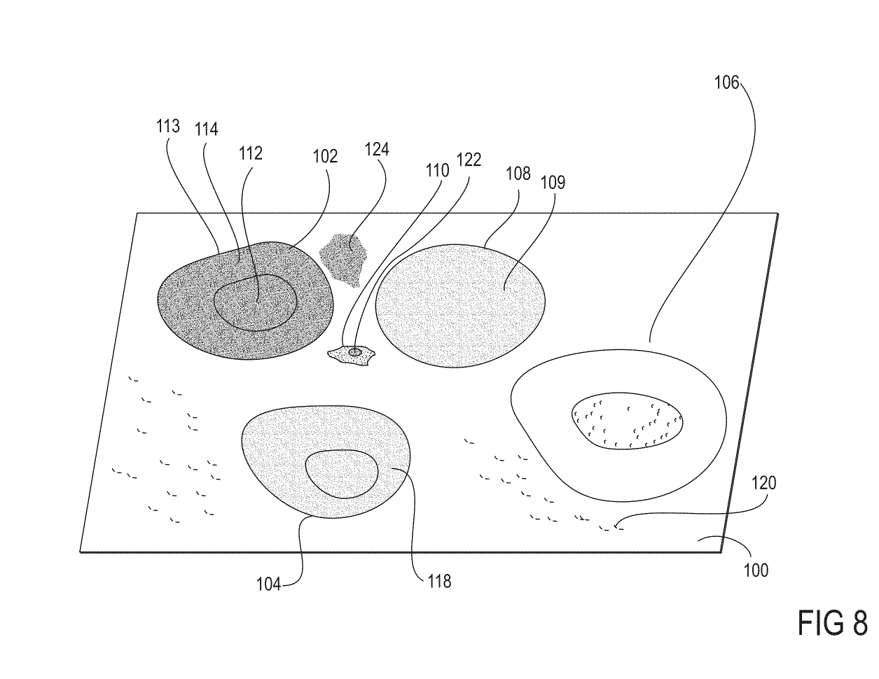

FIG. 8 shows an analysis of cells from a maternal blood sample. FIG. 8 shows slide 100 containing blood cells from a blood smear from a pregnant female according to the disclosure. The slide has been treated with an antibody and detection/staining reagents to detect fetal hemoglobin and is analyzed using an automated detector. Fetal red blood cell 113 with nucleus 112 contains a level of desired, hemoglobin specific signal 114, indicating the cell is a fetal red blood cell. Slide 100 also contains maternal red blood cell 104 which contains fetal hemoglobin and with a level of undesired, but hemoglobin specific signal 118. Maternal red blood cell 104 could be, for example, an F Cell from a pregnant female with hereditary persistence of fetal hemoglobin, sickle cell anemia, alpha thalassemia, or beta thalassemia. Slide 100 may also contain maternal red blood cell 108 having a low level of non-specific signal (staining) and white blood cell 106. Maternal red blood cell 108 may alternatively have no signal (staining). Substrate 100 may have a first level of non-specific signal 120, a second level of non-specific signal 124 or, may have no staining. Substrate 100 may also contain debris or junk 110 with unwanted non-specific signal 122. In some cases. Non-specific signal may be may be stronger than a specific signal and may interfere with analysis of fetal cells. Interference may be significant when an automated detection system is employed.

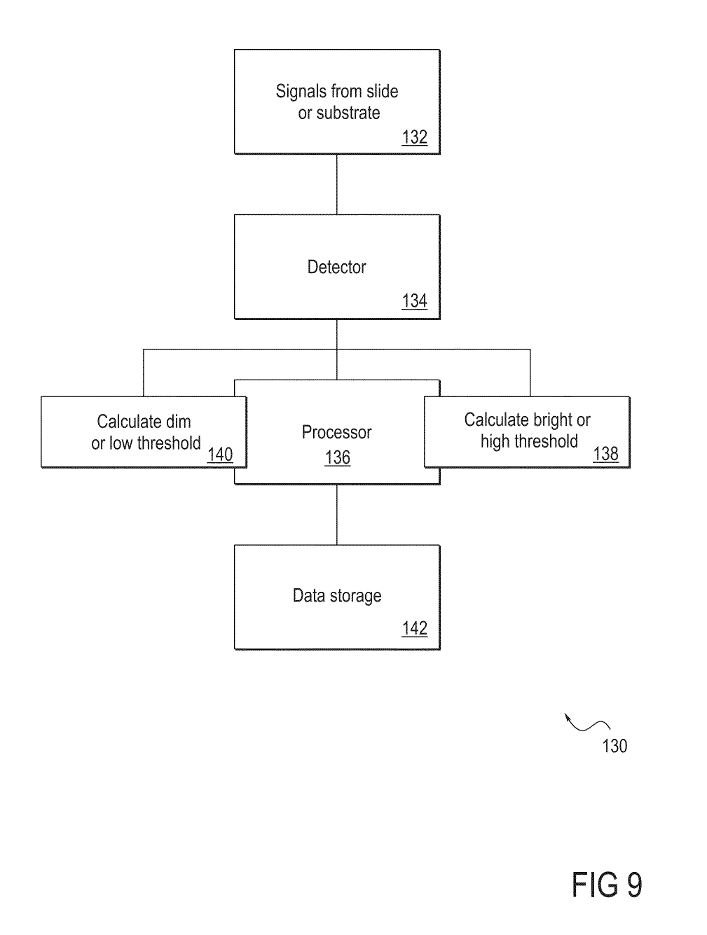

FIG. 9 shows steps in method 130 for detecting or distinguishing fetal cells on a cell smear from a pregnant female. Cells from a blood sample from a pregnant female are smeared onto a slide or substrate and treated with an anti-fetal hemoglobin antibody and treated (e.g. stained) to detect fetal hemoglobin, as shown in FIG. 8. The cells may also be stained with a nuclear stain, such as DAPI. Detector 134 detects a first signal from the DAPI treated nucleus and a second signal from hemoglobin detection treatment, as well as other specific and non-specific signals. Detector 134 detects DAPI specific signal from nucleus 112 from fetal cell 113 and no (or dim) DAPI signal from cytoplasm 114. Detector 134 also detects dim, specific signal 104 from maternal F cell 118. Detector 134 may also detect background 124 near fetal cell 113 from substrate 100 and/or different levels of background 120, 122 on substrate 100. Processor 136 calculates one or more signal intensity level(s) for the slide (or a portion of the slide) and one or more signal threshold value(s). A pixel(s), superpixel(s), or image(s) on the slide or substrate that is at or below a threshold value is removed from consideration, or flagged for further analysis. Locations on the slide and signal image values for the locations are stored in data storage 142.

It should be realized that each X, Y position on substrate 100, on a cell or on another item (e.g. debris) on substrate 100 can be detected and value(s) saved and analyzed. Average or compiled cell and substrate signal values, such as signal values shown in FIG. 8, can be calculated using methods and algorithms known in the art.

Any signal threshold value below a specific signal value of interest can be chosen as a cut-off level. An unwanted specific signal that is less than 1%, 5%, 10%, 15%, 20%, 25%, or 50% of a specific signal of interest may be removed from consideration or may be flagged for further consideration. The threshold signal level for the specific signal may be determined in any way. In one example, the threshold signal level for removing or flagging a specific signal is determined based on the total number of specific signals obtained (or as a percent of the total number of specific signals obtained). In one example, the threshold signal level may be chosen (at least in part) based on a fetal or maternal specific condition or risk factor (e.g. family history, medical diagnosis, symptoms). In one example, a threshold signal level of 15% is chosen if the pregnant female has sickle cell anemia.

The number of specific signals (e.g. cells) removed from consideration may be more than 1, more than 10, more than 100, more than 1000, or more than 10,000.

A signal or image (e.g. cell) that is removed from consideration may correspond to a pregnant female's cell. A cell may produce a fetal hemoglobin due to a condition or genetic status of the pregnant female for any reason, including, but not limited to hereditary persistence of fetal hemoglobin, sickle cell anemia, alpha thalassemia, beta thalassemia, leukemia, myeloproliferative disorders, other bone marrow disorders, or cancer.

The steps of the method may include calculating or using a first threshold signal brightness level and a second threshold signal brightness level. The method may include the step of removing a first signal that is below a first threshold level and/or the step of removing a second signal that is above a second threshold level.

The method according to the disclosure may include a step alerting, flagging, or reporting information about the specific signals. The alerting step may provide an alert that can be available to an analyst (e.g. medical provider) about the number of specific signals identified or the number of specific signals removed from consideration. The analyst may further confirm (e.g. manually) the specificity of the results or may communicate the results to the pregnant female or a health care provider. A high number of specific signals (e.g. 10,000) may indicate that the pregnant female has a bleeding problem that may need care or advice from a medical provider.

Although the method is described in detail for removing a dimmer or brighter signal or image (e.g. a cell) obtained from a slide treated to detect hemoglobin, the method can be applied to any analysis on a substrate or slide in which unwanted signals are obtained. The method can be used with a different surface, a different detection agent, or a different detection technique, including but not limited to a different hemoglobin antibody, a non-hemoglobin antibody, an RNA detection method such as in situ hybridization, and a glass, plastic, polymer, filter, paper or other substrate.

In some embodiments, a cell can be physically removed from the substrate and physically segregated from the population of non-nRBCs and other cells. In some embodiments, a cell and a portion of a substrate can be physically segregated from a population of non-nRBCs and other cells. The XY locations of the candidate nRBCs may also be used to control a microdissection system. Microdissection may be used to pick up candidate nRBCs and physically segregate them away from the non-nRBC cell population.

A cell may be separated from a substrate using any process or combinations or series of processes, and using any material that allows further use of the cell or some or all of a cell's content. A cell may be removed intact or may be removed in portions or only a portion of a cell (e.g. a nuclei or chromosome(s)) may be removed. A cell may be removed using mechanical means, including but not limited to abrasion, agitation, vacuum removal and scraping.

In some embodiments, a candidate fetal cell is removed from a substrate using a partial vacuum (a pressure lower than atmospheric pressure). A method of separating a candidate fetal cell from a maternal blood sample, may include the step of removing a candidate fetal nucleated cell from a surface comprising subjecting the cell to a pressure lower than atmospheric pressure to thereby remove the candidate fetal nucleated cell. For example, a candidate cell that is loosely attached or not attached to (e.g. is close to) a substrate may be removed using a micropipette. A candidate cell may be manually removed. Alternatively, a candidate cell may be automatically removed. For example, a processor and algorithm that recognize the XY location of a candidate nRBCs may be used to control a microdissection system to remove a cell.

A micropipette may be used remove (pick-up) a cell from the substrate and place the cell in a different location for additional analysis. A cell may be placed on a plate, on a substrate (e.g. a slide or membrane), in a well, in a tube, or any other suitable surface or vessel. Vacuum removal may be facilitated by treating the cell before, during, and/or after monolayer formation (as described above and below) with a treatment that aids in removal of the cell. In some embodiments, cells treated with bovine serum albumin prior to monolayer formation may be readily removed using a micropipette with a partial vacuum. A micropipette may have any size inner diameter and any size outer diameter as long as it can pick up a cell (which can vary in size, but on average have a diameter from about 5 μm to 8 μm) from the substrate (or pick up a cell and a substrate). A micropipette may have an inner diameter from 4 μm-10 μm, 10 μm-20 μm, 20 μm-30 μm, 30 μm-40 μm, 40 μm-50 μm or larger than 50 μm. In one example, a micropipette has an inner diameter less than 30 μm. Any part, but especially an inner portion of the micropipette may be coated with a material or solution to prevent a cell from sticking. For example, a micropipette may be coated with bovine serum albumin (e.g. less than 1%, less than 2%, less than 3%, or 3% or more than 3% (w/v). In one example, removing a candidate fetal nucleated cell comprises gathering the cell in a micropipette coated with less than about 1% (w/v) bovine serum albumin. A micropipette may be reconditioned during use (e.g. re-coated with additional BSA as indicated above).

A cell may be removed using chemical means, including but not limited to chemical treatment of a cell or chemical treatment of the substrate. A cell may be hardened or softened to aid in its separation. A cell and/or substrate may be subject to temperature change (e.g. cooling, freezing, or heating) to aid in the separation process. A cell might be loosened by changing the nature of the bonding interaction between the cell and the substrate. The substrate might be a simple glass surface or a modified glass surface. In some embodiments, a modified glass surface may lend itself to a more controlled removal of cells because of the specific chemistry of the interaction between the surface and the substrate. Modifications of the surface may include using or placing one or more polymer(s) including an adhesive(s) that can be more readily etched, hydrolyzed or solubilized than a glass surface. A polymer may serve the purpose simply to sufficiently weaken the cell substrate interaction that on the one had retained the cells on the surface through the labeling process, but on the other hand lessened the interaction as compared with a bare glass surface. In addition to (or instead of) polymers, the surface chemistry can be modulated by the introduction of self-assembled monolayers resulting in a non-silane surface. Either of these surfaces, polymeric or monomer, modified, can serve to assist in removal of cells by substituting a known specific covalent, ionic, or other binding interaction in place of a non-specific glass to cell interaction. A change to a specific binding chemistry would facilitate selective chemical removal since the specific binding chemistry is known. A specific polymeric or monomeric change might not only facilitate a specific chemical selectivity, the polymer or monomeric change could also (or instead) facilitate selective thermal desorption, photo-assisted desorption. Photo-assisted desorption may include either a photo-thermal desorption, such as used in some laser capture methods, or alternatively, a photochemical reaction that changes the bonding interaction between the cell and substrate by a change in bond order such as via free radical or ion radical chemistry. Examples of chemical functional groups that can influence bond interactions that can be introduced through monomeric or polymeric modification include, but are not limited to: thiol, acrylate, methacrylate, vinyl, epoxy, ethylene glycol, hydroxyl, isocyanate, pyridine, acrylic acid, amine, carbodamide, sulfone, and biotin. In addition to chemical means to soften or harden the cells prior to removal, polymers can be introduced to selectively encapsulate a cell prior to mechanical, chemical, thermal or photochemical, or photo-thermal desorption. The encapsulant may serve as a means of preserving the integrity of the cell during the course of separation.

A cell may be removed using a commercially available microdissection system such as a laser capture microdissection, e.g. a Zeiss Laser Microdissection System, an Applied Biosystems®Arcturus^{XT}™ Microdissection System, a Molecular Machines and Industries Laser Microdissection System, or a Leica Laser Microdissection System. However, in some cases a laser capture system designed for handling and removing a tissue sample may not work to remove or isolate a single cell. A single cell may have less weight (or mass) or maybe a smaller size compared with a tissue sample and may therefore behave differently.

In some embodiments, a cell and a portion of substrate to which it is attached may be removed together. They may be removed from the remainder of the substrate by any means. The means may include any of the processes and/or materials described above, or by another process such as by cutting a portion of a substrate such as a filter to remove it from the remainder of the substrate or filter. The substrate may be cut using a sharp object (e.g. a scissors or knife), a laser, or any other means.

A cell may then be removed from the substrate before additional analysis.

A fetal cell segregated from a substrate may be further analyzed separately. Alternatively, two or more segregated fetal cells may be combined and subjected to further analysis.

In some embodiments, a candidate fetal cell (or series of candidate fetal cells) is placed in a solution, fixed and analyzed. In some embodiments, a cell may be lysed. Proteins from the lysed cell may be analyzed, such as by ELISA (e.g. to determine fetal character), and the DNA may be subject to any of the analyses described herein.

One aspect of the invention provides a method for attaching a fetal cell to a surface, including placing a solution configured to maintain fetal cell integrity on a porous membrane; placing a cell in the solution on the membrane; and removing the solution and thereby adhering the cell to the porous membrane.

In one embodiment, a candidate cell (or a series of candidate cells) is removed from a substrate or surface and placed on a second substrate or second surface. A cell may be attached to the surface, removably attached to the surface, or settled or placed close to the surface (but not attached to the surface). Any cell from any source may be placed on the surface. A cell may be removed from a first surface (as described herein) or may be obtained from, for example, a FACS analysis, MACS analysis, a microfluidic analysis, or any other source. Although illustrated herein for fetal cells, the method and materials described herein may be used with any type of cell of interest, including a cell of interest and/or a rare cell, such as a stem cell or tumor cell.

Any of the materials described above (or a first substrate or surface may be used). In some embodiments, the second substrate or second surface may be a flexible membrane, a hydrophilic membrane, a porous membrane, and/or a resilient membrane. In some embodiments, placing a solution and/or placing a cell (e.g. a fetal cell or a candidate fetal cell) includes placing a solution and/or cell on a porous polyester membrane, such as e.g. polyethylene terephthalate (PET), biaxially-oriented polyethylene terephthalate (mylar) or polyethylene naphthlate (PEN).

A cell may be placed directly on the second substrate, which may include a small volume of solution removed from the first substrate. In some embodiments, an aliquot (drop) of a solution is placed on the second substrate and the cell is placed in the aliquot of solution. The aliquot may be a solution configured to maintain cell integrity. In some embodiments, a solution may be configured to lyse a cell and thereby expose the cell contents. Alternatively, the solution may be configured to maintain cell integrity. Any solution may be used. In some embodiments, phosphate buffered saline (PBS) is used to form the aliquot on the second substrate. In some embodiments, a solution (or the surface may include zinc (e.g. 5-50 mM, 30-50 mM, or 42 mM zinc sulfate). The solution placed on the second substrate may be less than 20 μl, less than 10 μl, less than 1 μl, less than 0.1 μl. In one example, a drop is between about 10 μl and about 50 fl. A solution may be placed on a membrane before or after a cell is placed on the membrane or may be placed at the same time. In one embodiment, a cell is in the solution and the steps of placing a solution and placing a cell are performed simultaneously.

One or more control cells may also be attached to the membrane. A control cell may have a known identity. A control cell may be any, such as a maternal red blood cell, a fetal red blood cell, a fetal red blood cell line, an embryonic red blood cell, an embryonic red cell line, a maternal white blood cell, a germ cell, a transfected cell and/or a cell or cell line, such as one expressing hemoglobin or expressing or having another marker. A control cell may express a fetal hemoglobin and be used as a positive control for anti-fetal hemoglobin antibody immunocytochemistry. A control cell may express an embryonic hemoglobin and be used as a positive control for anti-fetal hemoglobin antibody immunocytochemistry. A control cell may be identified as having X or Y chromosomes, or both

Some embodiments include repeating the placing a cell and removing the solution steps to thereby adhere a plurality of fetal cells to the surface. In some embodiments, each cell is separated from any other cell on the membrane. Having cells separated may allow better analysis and may make it easier to remove a single, isolated cell for subsequent use or analysis.

After placing the cell on the porous membrane, the cell may be adhered to the membrane. The cell may be adhered in any way, such as removing a solution or adding a solution or by a treatment. The solution may be removed in any way, such as by evaporating the solution. The solution may be evaporated at ambient temperature (around or at 68° C., or at a temperature above ambient temperature (e.g. by application of heat), or at a temperature below ambient temperature. In some embodiments, the membrane with the cells may be placed in a controlled environment, such as with less than 100% humidity, less than 90%, less than 80%, less than 70%, less than 60% or less than 50% humidity. 120. Some embodiments include having the porous membrane in a controlled environment comprising less than 100% humidity, or less than 70% humidity during at least a part of the removing the buffer step.

In some embodiments, the membrane may be placed in a controlled environment with ozone. Some treatments may slow the evaporation process, which may help maintain cell integrity and prevent cell lysis. One or more salts may be left behind. In one embodiment, the solution comprises phosphate buffered saline and evaporating comprises removing water from the phosphate buffered saline and leaving phosphate salts on the porous membrane.

Alternatively or additionally, a solution may be added that fixes the cell to the membrane. The solution may also fix the cell for further analysis. Alternatively, the cell may be attached by a treatment (such as an ultraviolet (UV) light fixation). A plurality of candidate cells may be similarly attached to a membrane. The cells may be adjacent to or touching one another or the cells may be separated from one another. Having separated cells may aid in an analysis and subsequent harvesting of a cell from the membrane.

Any type of porous membrane that will attach a cell and maintain genetic integrity to allow genetic analysis may be used. In some embodiments, a material with low background fluorescence is used; this allows fluorescent based analyses to be performed on a cell on a membrane. In some embodiments, a hydrophilic membrane is used, such as a UV treated membrane. In some embodiments, polyethylene naphthlate (PEN, biaxially-oriented polyethylene terephthalate or Mylar) and/or polyamine or poly-lysine surfaces are used. Other materials that hold a cell to the surface may be used (e.g. fibronectin, fibrin).

A candidate fetal cell or a plurality of candidate fetal cells on the porous membrane may be subject to fixation and further analysis. The cells may be fixed using any fixative, including any of the fixatives described herein and/or a fixative containing salt (e.g. sodium citrate), zinc (e.g. zinc sulfate), and/or a 2-bromo 2-nitrol, 3 diol related material. A cell on the membrane may be analyzed for any purpose. A cell may be analyzed to determine a genetic characteristic. A cell may be analyzed to determine or confirm fetal identity. A cell may be analyzed using in situ hybridization or by immunocytochemistry. In one embodiment, a cell or plurality of cells is analyzed using anti-fetal hemoglobin antibody and/or anti-embryonic hemoglobin antibody as described elsewhere herein to identify a fetal cell. In some embodiments, a cell may be analyzed using an anti-glycophorin, an anti-HLA-G, and/or anti-FECH antibody.

The contents or part of the contents of a cell (an organelle, a membrane, nucleic acid (any nucleic acid, including any listed herein), protein) may be removed from the membrane. For example, a cell may be lysed on the membrane and the contents removed such as with a pipette and/or using a vacuum source to another surface, membrane, tube or holder.

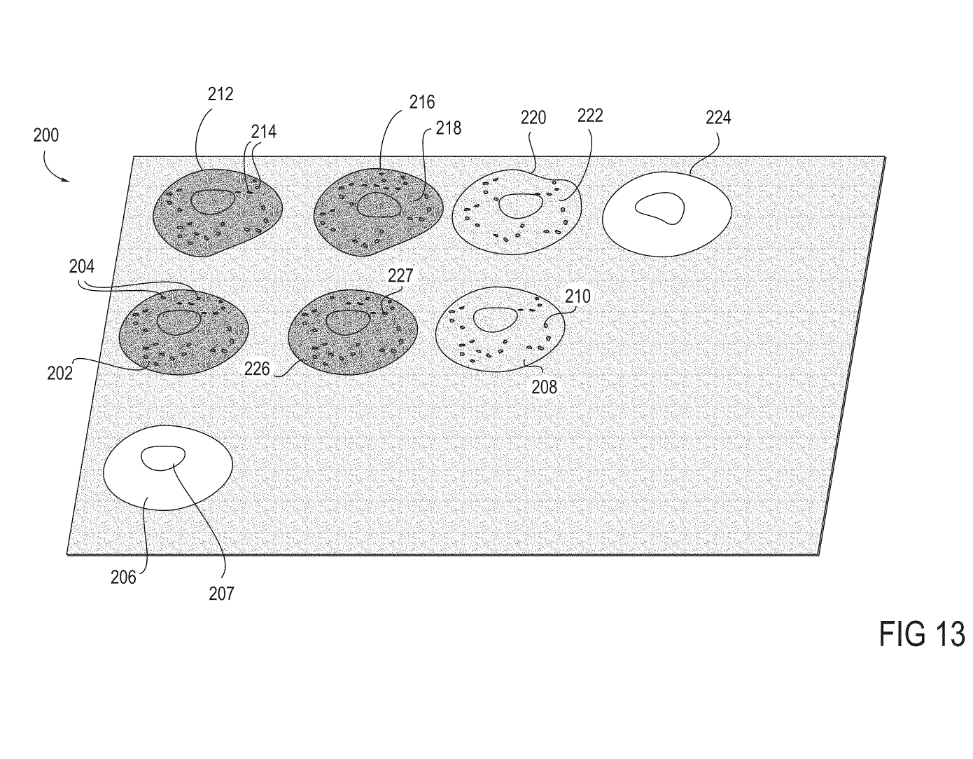

FIG. 13 shows methods and materials for analyzing cells on a porous membrane. Cells of interest (including potential fetal cells) have been removed from a monolayer of cells prepared from a maternal blood sample and placed on membrane 200 in an aliquot of a solution. (Membrane 200 may be considered a “second substrate” in this instance in which the monolayer substrate is a “first substrate”). The solution has been evaporated to thereby attach fetal cell 202, maternal cell 206 with its nucleus 207, and maternal cell 208 to the substrate. Control cell 212, control cell 214, control cell 216, and control cell 224 are also attached. Cells have been fixed on the membrane and immunocytochemistry performed using a combination of anti-fetal hemoglobin antibody and anti-embryonic hemoglobin antibody. Fetal cell 202 is positively stained, as shown by hemoglobin signal 204. Control cell 212 expresses embryonic hemoglobin and is positive for hemoglobin signal 214, such as for embryonic hemoglobin. Control cell 216 expresses fetal hemoglobin is also positive for hemoglobin signal 218, such as for fetal hemoglobin. Control cell 220 is an F cell that shows a relatively lower level of hemoglobin signal 222. Maternal cell 208 also shows a relatively lower level of hemoglobin signal 210, and is also an F cell. Neither maternal cell 206 nor negative control cell 224 show hemoglobin signal.

A cell of interest (e.g. a fetal cell or candidate fetal cell) may be removed intact or largely intact from the porous membrane or may be removed along with a portion of the porous membrane. A removed cell may be subject to any additional analysis. Any of the methods described herein for removing a cell from a substrate or surface may be used.

In one aspect of the invention, a method is provided for removing a cell (e.g. a fetal cell), including: providing a cell attached to a portion of a flexible, porous and/or resilient surface; apposing at least a portion of the flexible, porous, and/or resilient surface to a resilient substrate; surrounding the cell with a hollow shaft; and applying a force between the hollow shaft and the flexible surface wherein the force is opposed by the resilient substrate and/or a microscope stage or other hard (non resilient) surface, to thereby separate the portion of the flexible, porous, and/or resilient surface and the cell attached thereto from the rest of the flexible, porous, and/or resilient surface.

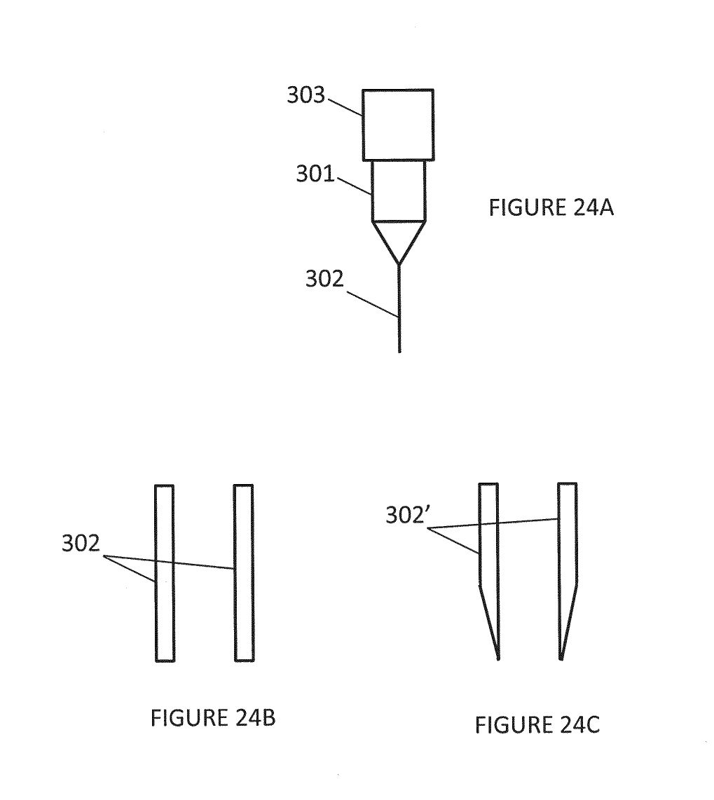

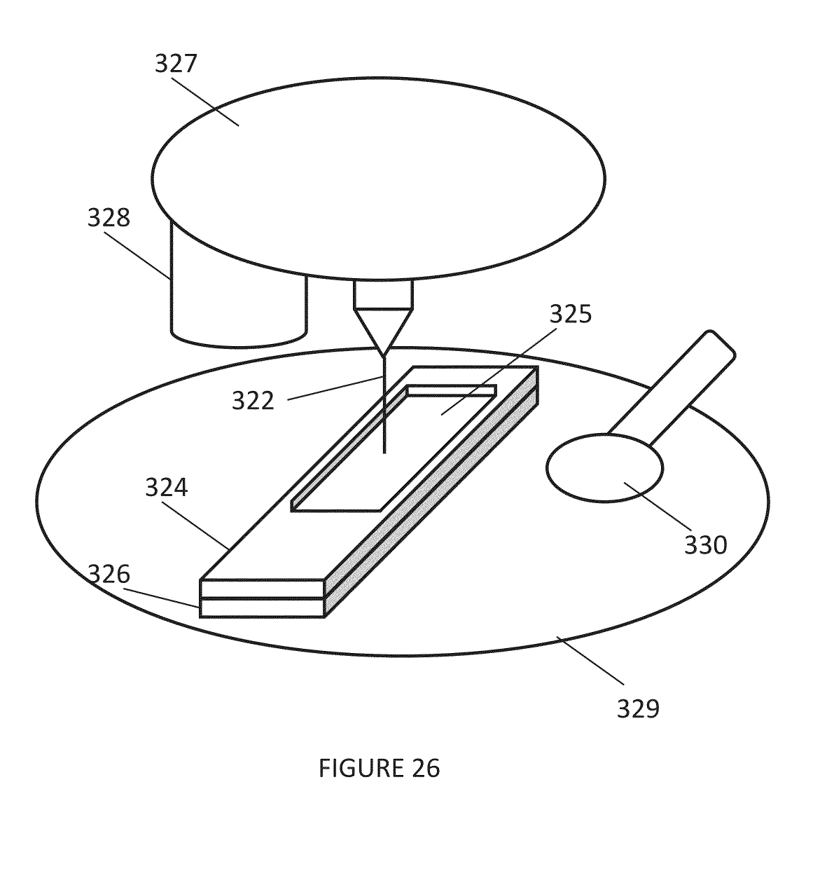

In one aspect of the invention, a device and system are provided for removing a cell. As shown in FIGS. 24A-C through FIG. 26, a cell separating device 301 may include a shaft having a first hollow end 302 and a second end 303. Hollow end 302 may be configured to remove a portion of a membrane. As shown, second end 303 may include a turret housing connected with the shaft and configured to connect the second end with a microscope objective turret. In general, the shaft is configured to remove a portion of a membrane when the turret housing is in place in a microscope objective turret and a flexible membrane is in place on a microscope stage 329.

As shown in FIG. 24A, device 301 for removing a cell may include hollow shaft 302. In some embodiments, removal device 301 may further include adapter 303. Adapter 303 may be configured to couple the removal device to a standard microscope. In some embodiments, the adapter may be configured to couple the removal device to an objective turret of a microscope. Alternatively, the removal device may be configured to connect directly to a standard microscope without the use of an adapter. In some embodiments, the hollow shaft is replaced and a new hollow shaft is used for each new cell to be removed and/or for each new sample of cells. In some embodiments, the adapter may be reused for multiple cells; alternatively, the adapter may be replaced for the removal of each new cell and/or the removal of cells from each new sample.

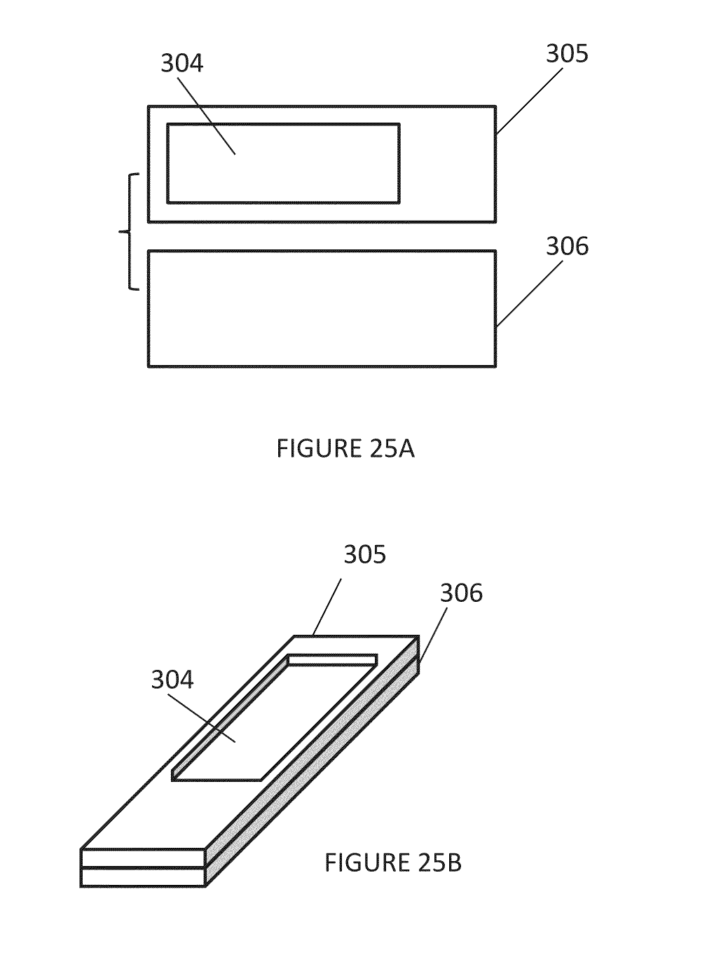

As shown in cross section in FIGS. 24B and 24C, hollow shaft 302 may be configured in one of several suitable configurations. As shown in cross section in FIG. 24B, hollow shaft 302 may have a blunt distal end. As described in further detail below, a force is applied between hollow shaft 302 and the flexible surface (314 in FIGS. 25A and 25B) wherein the force is opposed by the resilient substrate (316 in FIGS. 25A and 25B) and/or a non-resilient surface such as a microscope stage. Because hollow shaft 302 has a blunt distal end, the hollow shaft will punch a circle out of the flexible surface having an outer diameter about equal to the outer diameter of the hollow shaft. The hollow shaft will press the circular portion of the surface down onto the resilient substrate, such that the circular portion of the surface will not be pressed up into the hollow shaft. However, in some embodiments, a vacuum may be applied through the hollow shaft, such that the circular portion of the flexible surface (and the cell attached thereto) may be held against the distal end of the hollow shaft. Alternatively, tweezers or any other suitable grasping device may be used to pick up the punched portion of the flexible surface (and the cell attached thereto), from the surrounding flexible surface, such that the cell and portion of the flexible surface may be placed into a collection or sample tube, such as a PCR tube.

Alternatively, as shown in cross section in FIG. 24C, hollow shaft 302′ may have a sharpened end, resembling a leather punch. Again, as described in further detail below, a force is applied between hollow shaft 302′ and the flexible surface wherein the force is opposed by the resilient substrate and/or a non-resilient surface such as the microscope stage. Because hollow shaft 302′ has a sharpened distal end, the hollow shaft will punch a circle out of the flexible surface having an outer diameter about equal to the inner diameter of the hollow shaft. The hollow shaft will press through flexible surface and/or the resilient substrate such that the circular portion of the surface will be pressed up into the hollow shaft. In some embodiments, a single hollow shaft may be used for the removal of multiple cells. Thus, each of the cells may be pressed into the hollow shaft and retained there until all or a portion of the desired cells are collected. In this embodiment, a plurality of cells may be transported from the hollow shaft to a collection vessel such as a PCR tube.

In some embodiments, the inner diameter of the first hollow end of the hollow shaft 302 may be between about 200 um and about 400 um. In some embodiments, the outer diameter of the first hollow end of the hollow shaft 302 may be between about 300 um and about 500 um. In some embodiments, the inner diameter of the first hollow end is between about 100 um and about 300 um and an outer diameter of the first hollow end is between about 300 um and about 500 um and larger than the inner diameter. In some embodiments, shaft 302 may be a 25 gauge needle. In some embodiments, the shaft is stainless steel.

As shown in FIGS. 25A and 25B, the system for removing a cell may further include a resilient substrate 316 and a flexible surface 314. In some embodiments, the flexible surface 314 may be a porous membrane slide. In some specific embodiments, the flexible surface may be about 1 um thick. In some embodiments, the flexible surface may be a polyethylene terephthalate (PET) material, such as biaxially-oriented PET or MYLAR. Alternatively, the flexible surface may be a polyphenylene sulphide (PPS) material. In some embodiments, the material preferably has a low autofluorescence characteristic. Furthermore, a portion of the flexible surface is likely included with the cell of interest in the collection tube (e.g. PCR tube), therefore the material of the flexible surface preferably does not interfere with procedures and analyses (e.g. amplification and genetic analysis) performed on the cell. As shown in FIGS. 25A and 25B, in some embodiments, the flexible surface may be surrounded by a frame 315. Frame 315 may be a metal (such as aluminum), or any other suitable material. As shown in FIGS. 25A and 25B, the system for removing a cell may further include a resilient substrate 316 coupled to the flexible surface 314. The resilient substrate may be silicone or any other suitable resilient material.

As shown in FIG. 26, the system for removing a cell may further include a microscope system 327 and a feedback source 330. The microscope system may further include a camera 328 and a microscope stage 329. As shown in FIG. 26, flexible surface 324, frame 325, and resilient substrate 326 may be placed on, or otherwise coupled to the microscope stage 329. In some embodiments, the stage 329 is configured to move with respect to the microscope 327. For example stage 329 may be raised to bring the flexible surface 324 in contact with the shaft 322. Hollow shaft 322 maybe coupled to the objective turret of the microscope 327 as described above. Once the flexible surface is placed on the microscope stage, and the shaft 322 is coupled to the microscope, the shaft location may be calibrated with respect to the microscope. In one example, a sample punch may be performed by directing the shaft 322 against a location of the flexible surface. The location of the flexible surface may preferably not include a cell attached thereto. Once the sample punch is performed, the microscope 327 may be rotated such that the camera 328 is positioned over the flexible surface 324. A user may view the sample punch location on the flexible surface through the camera and may position the punch location in the center of the crosshairs as viewed through the microscope camera. The user may then select a button or otherwise indicate to a program, software, or microprocessor etc. that the current location is the location of the shaft, and where a punch will occur. Once the shaft location is calibrated with respect to the flexible surface, the cell removal process may begin. Preferably the location of the flexible surface is not changed during the cell removal process. In some embodiments, as described above, a new shaft may be used for each new cell to be removed. A new shaft may be used, for example, to prevent sample contamination. In some embodiments, the location of the shaft may be recalibrated for each new shaft coupled to the microscope.

Once the location of the shaft with respect the flexible surface is known by the system, a user may initiate the cell removal process. As described above, a flexible surface having at least once cell attached thereto is coupled to the microscope stage. In some embodiments, each cell adhered to the flexible surface may be separated from any other cells on the flexible surface. The cell removal process may begin with the step of examining through the microscope camera positioned over the flexible surface at least a portion of the cells on the flexible surface, looking for particular cells of interest. For example, a cell of interest may be a fetal cell. In a specific example, a cell of interest may be a fetal nucleated red blood cell. The cells of interest may be identified through any suitable process, examples of which are described herein. For example, a user may analyze a characteristic of the cell attached to the surface before the surrounding step (e.g. surround the cell of interest with the shaft 302). In some embodiments, analyzing a characteristic of the cell may include performing at least one of a DNA analysis, an RNA analysis, a protein analysis, and an optical analysis prior to the applying step. In some embodiments, performing a protein analysis may include performing immunocytochemistry. For example, performing immunocytochemistry may include analyzing at least one of fetal hemoglobin and embryonic hemoglobin.

With the camera 328 in position over the flexible surface, a user may move the stage from cell to cell. Alternatively, the system may automatically move the stage from position to position. Once a cell of interest is identified, a user may rotate the microscope 327 such that the shaft 322 is positioned over the cell of interest. Alternatively, the system may automatically rotate the shaft over the cell of interest. Once the shaft is in position the stage may be raised to bring the flexible surface in contact with the shaft. The stage may be raised until the portion of the flexible surface is punched from the surrounding flexible surface. The stage may be stopped by a user, or by the system automatically upon receiving feedback that the punch has been completed.

Feedback source 330 may be one of several suitable embodiments. For example, feedback source may be a microphone, or other suitable electronic vibration sensor, configured to pick up the audible indication, e.g. a sound such as a “snap”, that the punch has been completed. In some embodiments, the microphone may be coupled to the microscope stage. In some embodiments, the microphone may be coupled to the flexible surface or frame directly. In some embodiments, the microphone may be coupled to an amplifier with automatic gain control. For example, the frequency characteristics may be adjusted to receive a focused band of frequencies detected with the “snap” sound as described above. For example, a band-pass filter may be used to pass frequencies within the range of the “snap” sound and reject or attenuate frequencies outside of that range. In some embodiments, the “snap” may be magnified such that it can be heard clearly by a human ear. Alternatively, the sound may be converted to a digital feedback signal that may be fed back to the system controlling the motorized stage. The system may automatically stop raising the stage and/or begin lowering the stage upon receiving the digital feedback signal.

In an alternative embodiment, the feedback source may be provided through a strain gauge coupled to the shaft 322. In this embodiment, the strain gauge would detect an abrupt lowering in force or pressure felt by the shaft upon completion of punching through the flexible substrate. In an additional alternative embodiment, the feedback source may be provided optically by a laser. A laser, such as a laser displacement sensor, may be used to sense the compression of the flexible surface and/or the resilient substrate and the release of that compression upon the completion of the punch.

Upon completion of a punch around a cell of interest, the (circular) portion of the flexible surface and the cell attached thereto may be retrieved from the surrounding flexible surface and placed in a solution configured to perform at least one of detaching the cell from the membrane and extracting DNA from the cell. The steps described above may be repeated to obtain a plurality of cells of interest. In some embodiments, DNA may be obtained from the plurality of cells, the DNA may be amplified using PCR, for example, and an average amplified DNA yield per cell of at least 0.35 μg when using less than 36 cycles of PCR may be obtained.

In some embodiments, DNA quality and DNA yield must be maintained during various steps of the process, such as obtaining a maternal blood sample, enriching the maternal blood sample to obtain maternal cells and at least one fetal cell, creating a monolayer from the maternal blood sample, characterizing the cells, obtain a separate fetal cell, and further analyzing the cell. In some embodiments, PCR may be performed as part of an analysis or characterization of a fetal cell in order to obtain sufficient material. In other cases, performing a PCR based analysis may serve as a surrogate for DNA quality.

Some embodiments include repeating steps of the method (e.g. the providing, opposing, surrounding, and applying steps) to obtain a plurality of cells; obtaining DNA from the plurality of cells; and amplifying the DNA using PCR to obtain amplified DNA; and obtaining an average yield per cell of at least 0.20 μg, at least 0.25 μg, at least 0.30 μg, at least 0.35 μg, or at least 0.40 μg amplified DNA when using less than 36 cycles of PCR, less than 35 cycles of PCR, less than 34 cycles of PCR, less than 33 cycles of PCR, less than 32 cycles of PCR, less than 31 cycles of PCR, or less than 30 cycles of PCR. Amplification may be performed using, for example, a Rubicon Genomics kit, such as PicoPlex. and the DNA yield may be measured using a Nanodrop spectrophotometer.

A cell separating device for separating a cell (and portion of a surface) may include a hollow shaft configured to surround a cell (or a group of cells) and to apply a force to separate a portion of a membrane. A hollow shaft may be hollow on one end (e.g. have an indented end) or may be hollow from a first end to a second end. A shaft may be hollow to allow visualization therethrough, such as from a microscope to a cell on a flexible surface. A first end (e.g. a membrane contacting end) may be substantially flat (perpendicular) or may be beveled (e.g. beveled from an inner diameter to an outer diameter) and/or otherwise shaped (e.g. jagged, serrated, sharpened, toothed). A bevel may be angled in or angled out. In some embodiments, an end may mate with a mating structure on a resilient substrate, such as to aid in separating a cell and/or portion of a surface from the rest of the surface. A hollow shaft may take any shape as long as it is able to remove a portion of a membrane. For example, a hollow shaft may be substantially cylindrically shaped, or may be tapered from a first hollow end to a second end. A first hollow end of a shaft may be made of any material that provides sufficient strength to remove a portion of a membrane. In one example, the first hollow end comprises stainless steel.

In some embodiments, a cell separator may be configured to be hand-held. In some embodiments, a cell separating device may be connected to a microscope or may be configured to connect with a microscope, such as via turret housing to an objective turret. In one aspect of the invention, a cell separating device may include a shaft with a hollow end having an inner diameter and an outer diameter; and a turret housing connected with the shaft and configured to attach the shaft with a microscope objective turret wherein the shaft is configured to remove a portion of a membrane when the turret housing is in place in a microscope objective turret and a flexible membrane is in place on a microscope stage.

Any microscope as known in the art may be used. Some non-limiting examples of microscopes that may be used are optical microscopes such as those made by Bausch and Lomb, Celestron, Leica, Olympus, etc.

A resilient substrate may be any substrate under the surface that provides sufficient support to oppose a force cutting the surface. In one embodiment, the substrate is a thick silicone layer.

In some embodiments, a cell is separated from any other cells on the surface and one cell is removed using the hollow shaft. In some other embodiments, two cells, three cells, four cells, five cells, between five and ten cells, ten cells, or more than 10 cells may be removed simultaneously (e.g. using the hollow shaft). The process may be repeated to obtain a plurality of cells. In some embodiments, a cell that is removed (e.g. a cell of interest) may be an unwanted cell (e.g. may be a maternal cell if fetal cells are desired) and all or essentially all of the unwanted cells may be removed to thereby leave a membrane containing only desired cells (e.g. fetal cells), such that the entire membrane may be subject to further analysis.

In some embodiments, a method of removing a cell may include a step of analyzing a characteristic of the cell attached to the surface before performing the surrounding step or the applying a force step, and may further include determining a cell to be removed based on the cell characteristic. Any characteristic of a cell may be analyzed and any analysis method may be used, such as a DNA analysis, an RNA analysis (including but not limited to in situ hybridization), a protein analysis (including but not limited to immunocytochemistry including analyzing hemoglobin such as fetal hemoglobin and/or embryonic hemoglobin), and an optical analysis.

In some embodiments, a method of removing a cell may further include placing the portion of the surface, substrate, or membrane and the cell attached thereto in a solution configured to perform at least one of detaching the cell from the membrane and extracting DNA from the cell.

Any steps involved in analyzing and removing a cell may be controlled by a microprocessor and may be automatically performed. In one embodiment, the steps of surrounding a cell with a hollow shaft and applying a force may be automatically controlled.

In some embodiments, a cell may be placed on a second porous membrane and subject to an additional analysis. In some embodiments, a nucleic acid (DNA, such as genomic DNA (gDNA), mitochondrial DNA, antisense RNA, micro RNA (miRNA) messenger RNA (mRNA), non-coding RNA (ncRNA), piwi-interacting RNA (piRNA), ribosomal RNA (rRNA), small interfering RNA (siRNA), transfer RNA (tRNA) may be extracted, isolated, or otherwise obtained from a cell.

A method for determining a genetic status of a fetus may include providing a sample of maternal blood; enriching nucleated red blood cells including both fetal and maternal nucleated red blood cells in the maternal blood sample; and differentiating the fetal red blood cells from all other cells or from all other blood cells in the enriched sample. The method may further include separating at least one fetal cell from a remainder of the sample. The method may include combining two or more isolated fetal cells.

A microdissection enrichment process may be used to provide high purity DNA samples for use in microarray applications (e.g. array comparative genomic hybridization, and/or PCR (e.g. single molecule (smPCR), digital PCR (dPCR), quantitative PCR (qPCR)), sequencing or other DNA amplification and/or analysis methods.

Genetic testing and fetal/maternal differentiation can now be performed. Standard FISH, PCR (e.g. dPCR or qPCR), and/or array or microarray analysis (e.g. array comparative genomic hybridization) may be performed on the nRBCs that are found using the automated cell identification algorithms.

The separated fetal cell(s) may be further analyzed in any way. A single cell may be analyzed, or groups of cells (e.g. 2-10, 11-50, or more than 50) may be combined for further analysis. Two or more cells or two or more groups of cells may be analyzed and the results compared. In some embodiments, a group of fetal cells for analysis may include one, two or more maternal cells in a group of 10 cells; data corresponding to the maternal cell may be identified during the final analyses and removed from consideration. In one example, a fetal cell may be analyzed (including obtaining, analyzing, and/or amplifying the DNA or RNA from the at least one fetal cell) using PCR (e.g. dPCR or qPCR, RT-PCR), in situ hybridization, array or microarray analysis, including array Comparative Genomic Hybridization, and/or sequencing. In one embodiment, both arrayCGH and sequencing are performed.

DNA from a cell or a group of cells may be amplified by PCR. High quality DNA may be required for some analyses. For example, DNA may need to non-crosslinked or may need to be a sufficient length and of a sufficient yield. In some embodiments, quality of DNA may be analyzed, such as by optical analysis and may include other steps such as PCR amplification. For some embodiments, very high quality DNA is obtained. In some embodiments, DNA from a fetal cell or a group of fetal cells obtained by any method or combination of methods described herein is amplified by PCR and a characteristic of the DNA analyzed. Some embodiments include obtaining an average amplified DNA yield per cell of at least 0.35 ug when using 36 cycles of PCR.

DNA from a cell (or a group of) fetal cells may be analyzed for fetal character. In some embodiments, the method includes amplifying a first portion of fetal DNA for less than 6 cycles from each potential fetal cell of a plurality of cells, analyzing the DNA using a fetal identifier and obtaining samples positive for the fetal identifier. The method may include pooling and amplifying a second portion of DNA from the plurality of cells to obtain pooled, amplified DNA. The DNA may be analyzed for the presence of fetal specific sequences. In one embodiment, the DNA is analyzed for Short Tandem Repeats (STRs). A kit such as a Promega STR Analysis kit (PowerPlex) may be used.

In one embodiment, a plurality of fetal cells (e.g. around 10) are amplified by PCR. A custom protocol and materials may be used or an available kit may be used (e.g. Rubicon Genomics PicoPlex) to obtain sufficient DNA for arrayCGH analysis, sequencing or other analyses. Any type of array may be used for performing arrayCGH. An off-the-shelf array (e.g. Agilent, Affymetrix, Life Technologies Inc., etc.) using processes know in the art may be used or a custom array may be used. A reference DNA for analysis may be from one individual (maternal DNA, paternal DNA, a sibling, another relative, any of whom might or might not have a genetic status of interest) or may be from a group of individuals, including any of the aforementioned individuals.

A fetal cell or cells may be analyzed for any genetic status, disease, or condition, including but not limited to gene, partial or whole chromosome copy number changes (copy number variations, CMV) including but not limited to deletions or duplications (including repeats/tandem repeats) of portions of genomic DNA); (e.g. aneuploidy, trisomy, tetrasomy) including trisomy 1, 2, 3, 4, 5, 6, 7, 8 (Warkany Syndrome), 9, 10, 11, 12, 13 (Patou Syndrome), 14, 15, 16, 17, 18 (e.g. Edward syndrome), 19, 20, 21 (Down Syndrome), 22, XY ((Turner Syndrome; single X); Kleinfelters, Triple X, XYY); deletions, translocations, inversions, rings; detection of a single nucleotide polymorphism (SNP); mosaicism. In one example, one or more genetic status or condition selected from T13, T18, T21, XY, and cystic fibrosis, Tay-Sachs, sickle cell, Duchenne muscular dystrophy, and hemophilia are analyzed. In another example, one or more genetic status or condition selected from T13, T18, T21, and XY are analyzed.

Kits are provided for performing the methods described herein and/or comprising the components (including compositions) described herein. Any of the components (including compositions) may be combined with any others in a kit.

A kit may be provided for enriching for fetal nucleated cells from a blood sample using a filter system for enriching for fetal nucleated cells as described herein. The kit may include one or more of the components (including compositions) described herein. The kit may include an instruction for performing the method for enriching for fetal nucleated cells from a blood sample using a filter system. In one example, the kit includes a filter and an elution buffer. The elution buffer may comprise trehalose and maltose. In another example a kit may include an instruction for performing the method including the steps of passing a maternal blood sample containing maternal nucleated cells and fetal nucleated cells through a filter; retaining nucleated cells on the filter; and eluting nucleated cells from the filter with an elution buffer wherein the nucleated cells include fetal nucleated cells. In some embodiments, the kit may further include a pump for delivering the elution fluid through the filter. For example, as described above the pump may be used to deliver a predetermined volume of elution fluid through the filter in a predetermined amount of time.

Kits are provided for creating a monolayer of cells on a surface. A kit may include a smear blade with a flexure and/or a cell dilution and/or resuspension solution. The kit may include one or more solutions for fixing and washing the cells. The kit may include an instruction for performing a method, the method including the following steps: moving a sample of cells in at least two directions relative to a surface with the smear blade to create a monolayer of cells on the surface; and adhering the cells to the surface.

Kits are provided for testing a sample to identify a fetal cell. A kit may include anti-fetal hemoglobin (gamma, and/or zeta) and/or anti-epsilon hemoglobin and may include an instruction for use. In one example, the instruction may describe a method for testing a sample to identify fetal cells. The instruction may include any of the method(s) described herein. The kit may further include one or more agents to aid in detecting the antibody (e.g. horseradish peroxidase, alkaline phosphatase, Alexa Fluor 488 tyramide). The kit may further include a nuclear detecting agent (e.g. a nuclear stain, including but not limited to DAPI). The kit may include a composition (e.g. a buffer) for preparing the cells and/or a composition (e.g. a buffer) for washing the cells. The kit may include one or more solutions for fixing and washing the cells.

### EXAMPLES

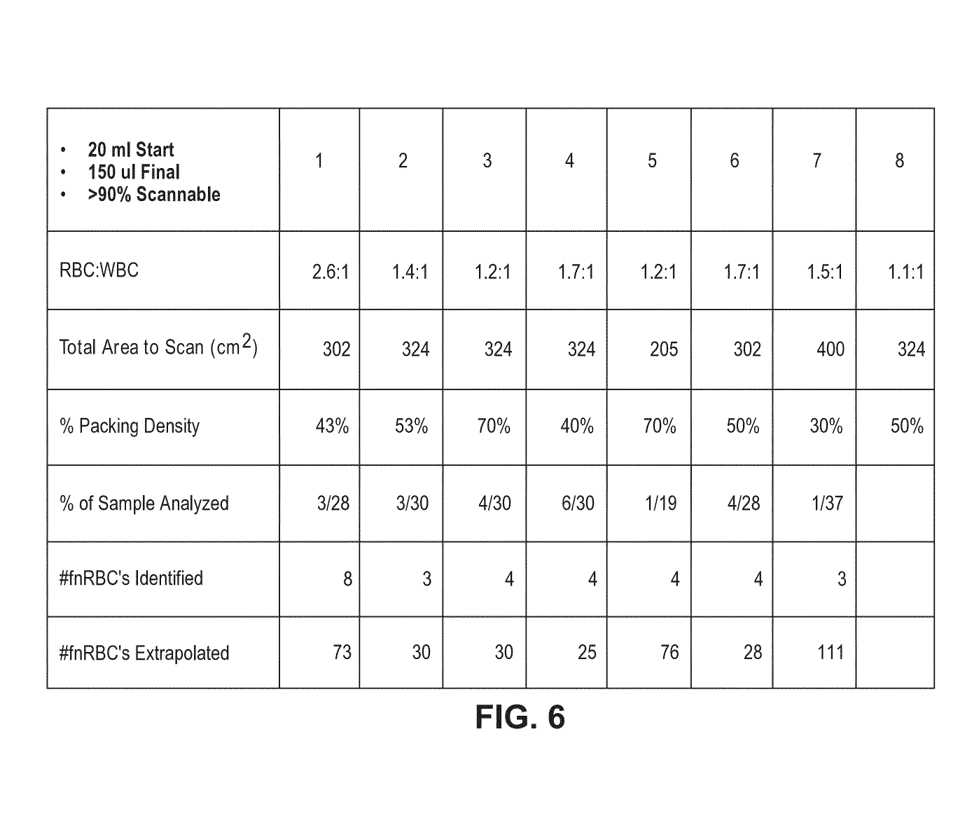

FIG. 6 shows data obtained from maternal blood samples after enrichment for fetal nucleated cells according to one embodiment of the disclosure. The maternal blood samples were passed over leukocyte depletion filters and cells remaining on the filter were eluted using elution buffer as described above. The cells were smeared onto slides and stained with DAPI to detect nucleated cells and subject to immunohistochemistry using anti epsilon hemoglobin antibody to detect the presence of fetal cells. A portion (% of sample analyzed) of the slides were illuminated with 420 nm and UV light to distinguish nucleated from non-nucleated cells, and cells with hemoglobin from cells without hemoglobin. Cells were classified as non-nucleated red blood cells (RBC), white blood cells (WBC), and candidate nucleated red blood cells and the ratio of red blood cells to white blood cells (RBC:WBC) in the sample calculated. A % packing density was calculated based on the total number of cells counted. Fetal nucleated Red Blood Cells in the fields analyzed was confirmed using anti epsilon hemoglobin antibody (#fnRBCs identified), and the predicted number of fetal nucleated red blood cells (#fnRBCs extrapolated) in each sample extrapolated.

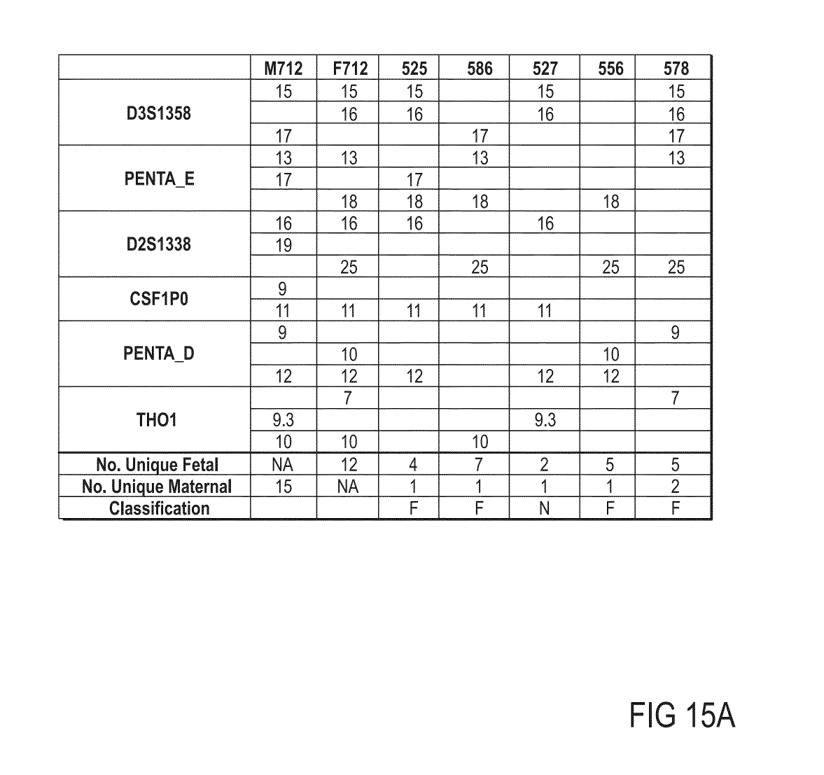

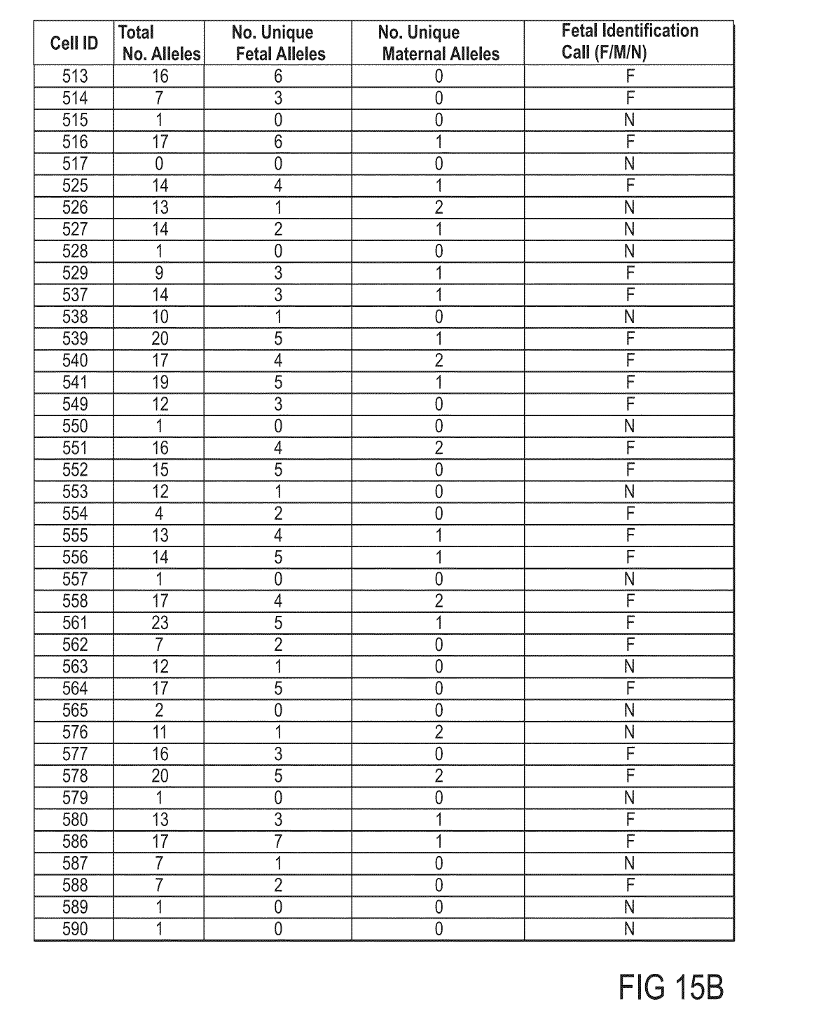

FIGS. 15A-B show results of a short tandem repeat (STR) analysis to identify fetal cells as fetal cells following the disclosure. Short tandem repeats are repeating sequences of DNA found in the genome. An STR can vary between two (or more) individuals but can also be the same. Finding variations in a number of different STRs between two samples means that the samples are different from each other. Each variation can be considered an allele. A series of STRs were used in this experiment to determine if single fetal and single maternal cells from a maternal blood sample could be distinguished from each other and be used to assign “fetalness” to a cell. A representative sample of STRs are shown in FIG. 15A. A monolayer of cells was created on a substrate from a maternal blood sample, and analyzed for the presence of hemoglobin and nuclei. Positive cells were individually removed from the substrate. DNA from the cells obtained by the methods described herein was amplified using a whole genome amplification kit from Rubicon Genomics using a limited number of cycles (e.g. 6). A panel of STRs was then PCR amplified using a Promega STR analysis kit from this whole genome amplified DNA as well as from DNA extracted from known fetal tissue (F712) and known maternal tissue (M712), and the results analyzed.

FIG. 15A shows representative data from a subset of samples and a subset of STR markers. The alleles identity is shown on the left. The results for individual cells can be compared with the results for the fetal and maternal tissue. Note that the number of unique alleles and unique maternal alleles is a summary of all results and is not just from the representative data shown from the subset of STR markers. FIG. 15B shows results from individual cells, including the total number of alleles identified, and how many were unique fetal alleles and how many were unique maternal alleles. (Other alleles would be shared and not used in this analysis for determining fetal character). Based on these results, a cell was identified as fetal (F), maternal (M), or not identified (N). The total number of alleles varies between samples due to technical limitations, such as stochastic amplification events, DNA quality, etc.

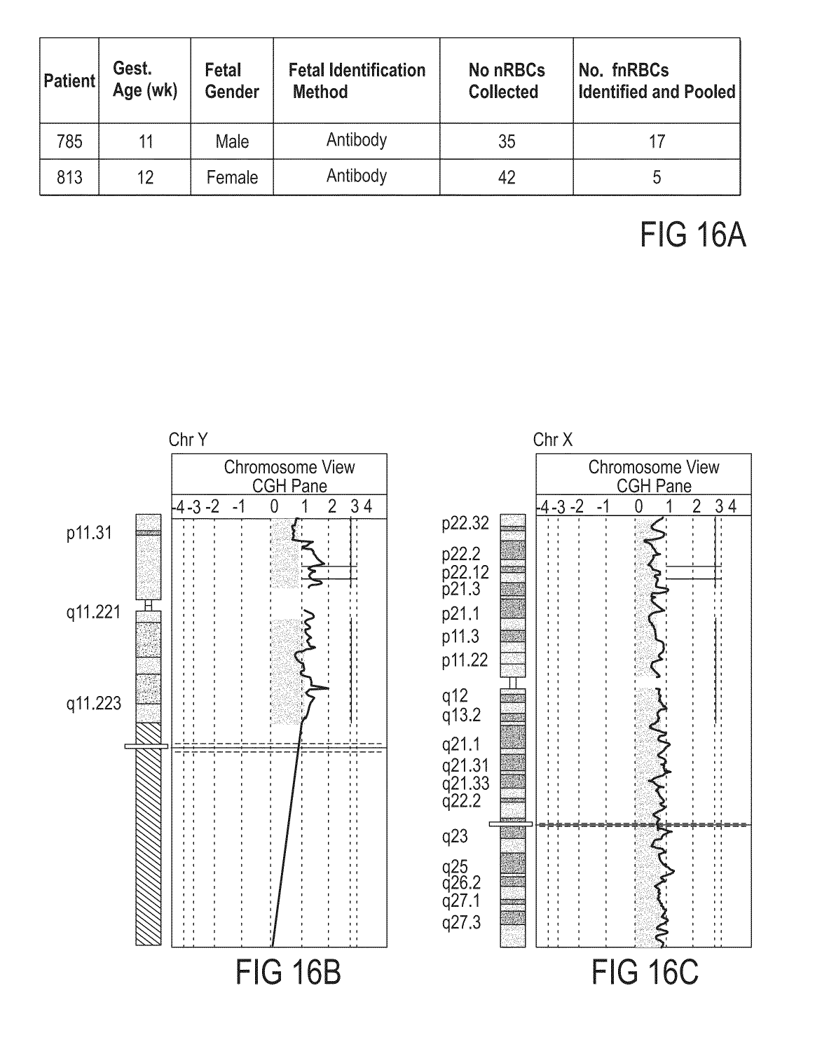

FIGS. 16A-C show results of an array Comparative Genomic Hybridization analysis to identify fetal characteristics from different samples of pooled fetal cells from two individuals according to the disclosure. Two separate samples for which sex had been otherwise determined were analyzed. Briefly, a self-assembling monolayer was created from the maternal blood sample (for each), the monolayer was analyzed using 420 nm light to identify hemoglobin containing cells and fluorescence to detect nuclei. Individual cells (35 in the first sample and 42 in the second sample) showing a concurrence of hemoglobin and fluorescence were removed from the monolayer and transferred to a flexible membrane. The cells were analyzed by immunocytochemistry using anti-fetal hemoglobin and anti-embryonic hemoglobin antibodies to detect hemoglobin and identify cells as fetal. Identified fetal cells were pooled and amplified by PCR using whole genome amplification. The amplified fetal DNA and a reference sample were separately labeled with different color dyes and the samples analyzed by Comparative Genomic Hybridization on an Agilent array. A reference DNA was chosen as a gender mismatch (e.g. female reference sample for a male fetus) in order to analyze differences between the samples. Results are shown for the Y (sample 785) and X (sample 813) chromosomes in the ideograms in FIGS. 16B and 16C.

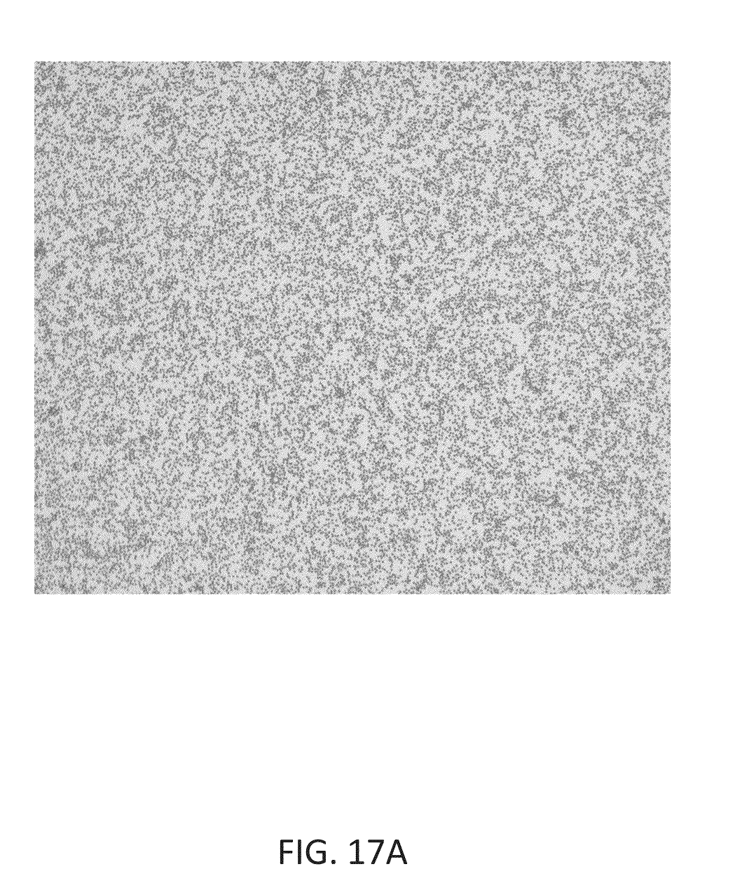

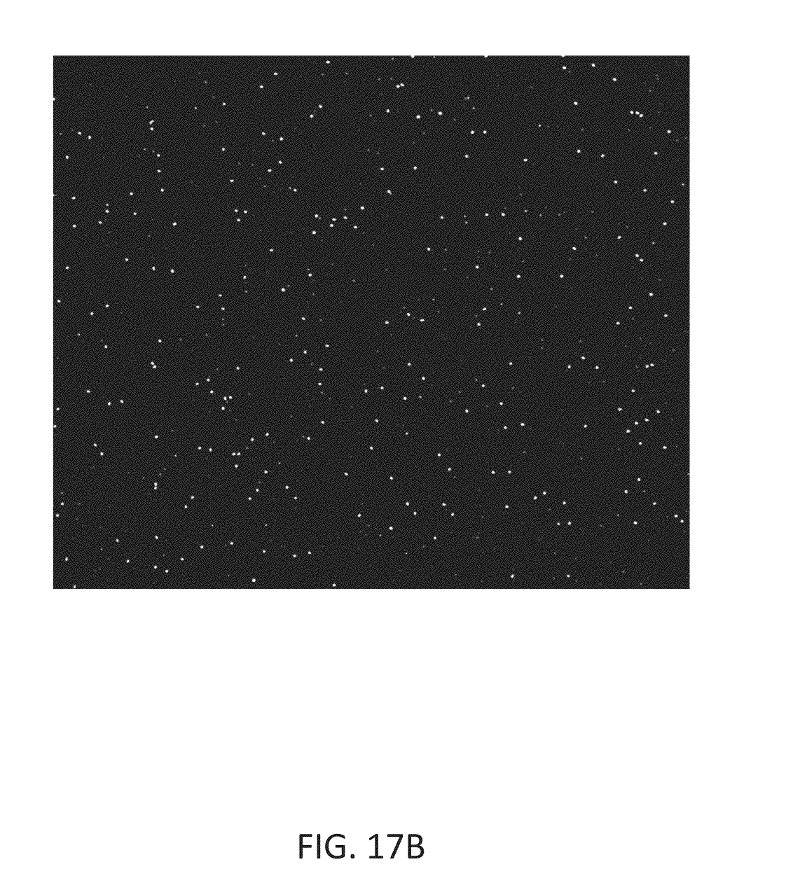

FIGS. 17A-B show one field of view of a monolayer of cells illuminated using 420 nm LED light to identify hemoglobin containing cells (FIG. 17A) and analyzed by fluorescence for nuclei using SYBR Green (FIG. 17B). Hemoglobin containing cells are red blood cells. Nucleated cells include maternal white blood cells and nucleated red blood cells.

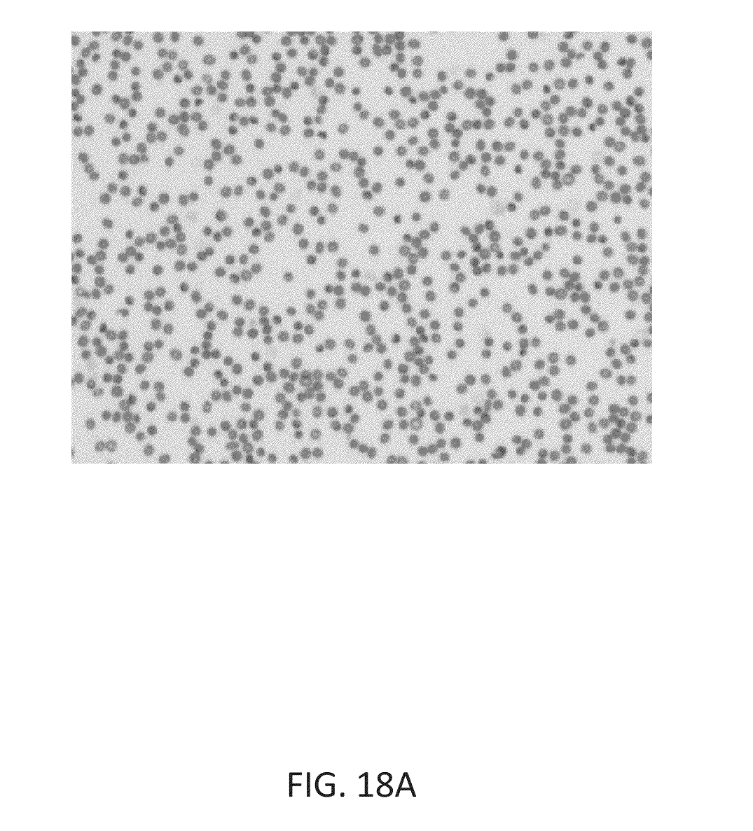

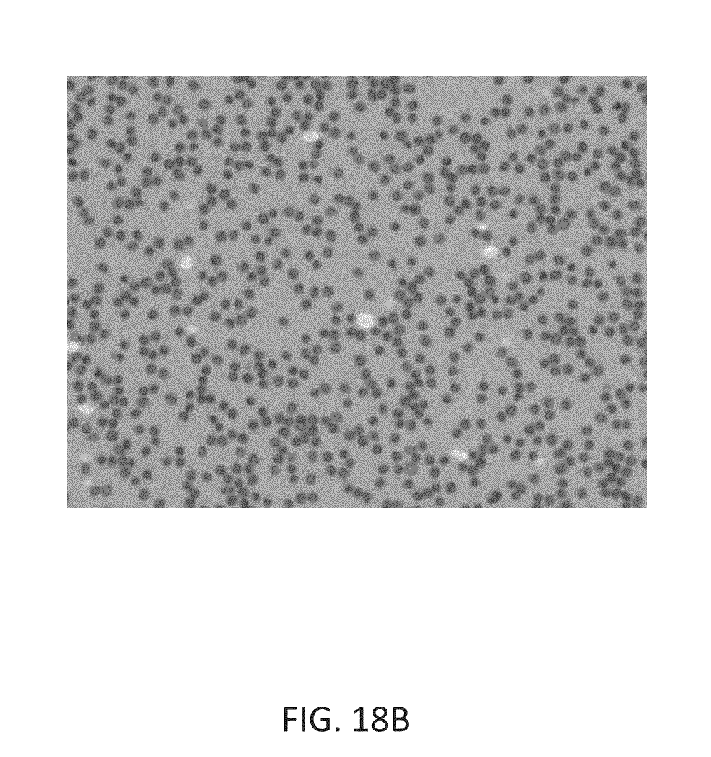

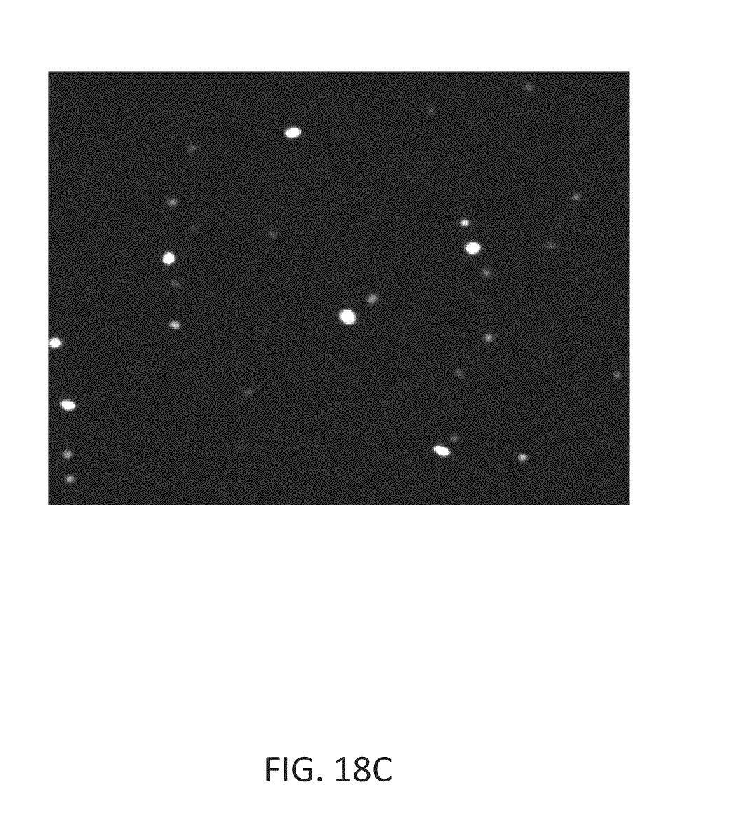

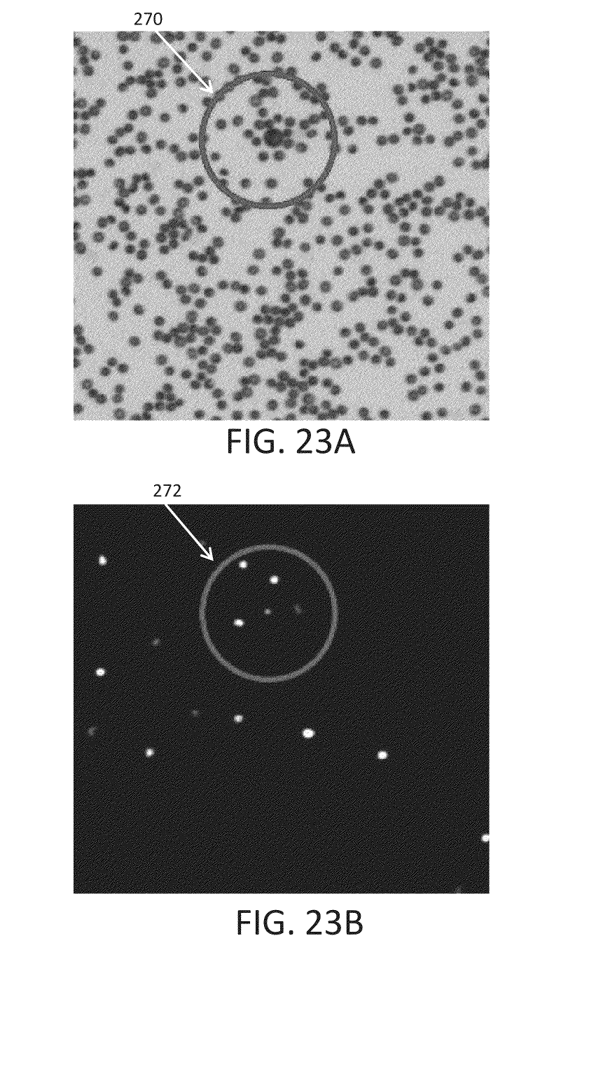

FIGS. 23A-B show two images of a monolayer from a maternal blood sample analyzed for hemoglobin (FIG. 23A) and for the presence of nuclei (FIG. 23B). Circle 270 shows a hemoglobin positive cell at its center. The same cell 272 is shown detected for a nuclear signal in FIG. 23A. When two signals are present in a single cell, they may affect or interfere with each other relative to other cells and the signals may appear relatively stronger or weaker than expected when imaged. FIGS. 18A-C show a detail view of a portion of the monolayer shown in FIGS. 17A-B using 420 nm light hemoglobin (FIG. 18A), fluorescence for nuclei (FIG. 18B), and both (FIG. 18C). Depending on the illumination and imaging conditions, a nuclei may appear larger than its expected size if compared with another image of the same cells analyzed under different conditions.

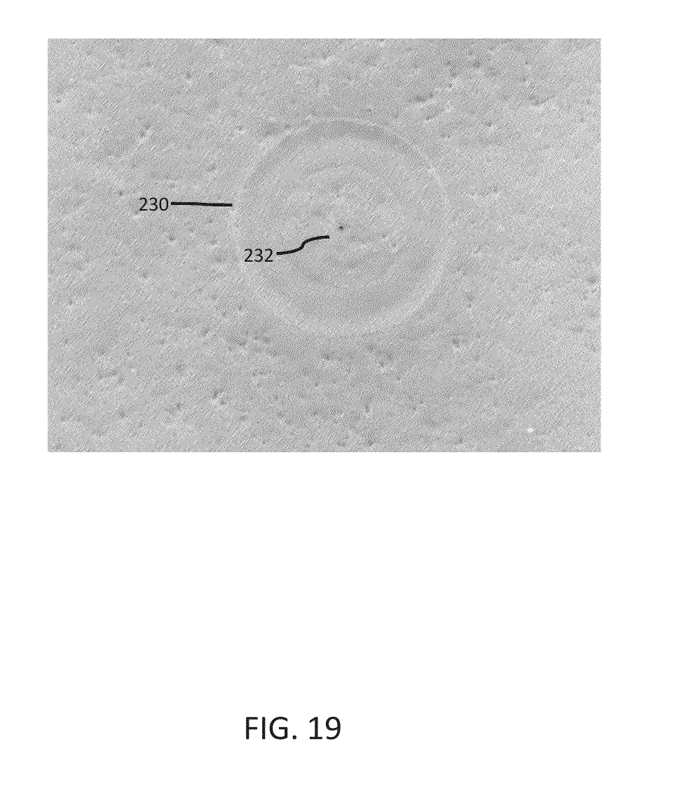

FIG. 19 shows a cell 232 identified by an illuminated nuclei and a circular portion of a membrane 230 being removed from a flexible membrane according to one aspect of the disclosure. The cell and portion have been removed using a punch according to one aspect of the disclosure. The portion of the membrane being removed is about 400 um in diameter.

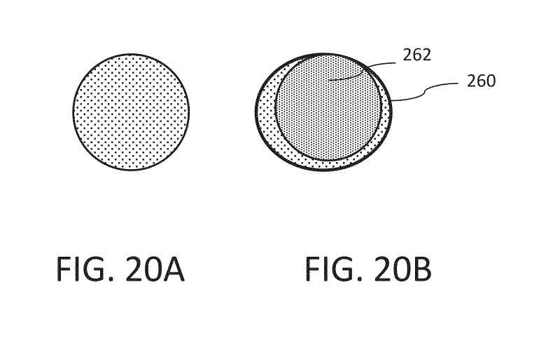

FIGS. 20A-B shows cells obtained from a monolayer from an enriched maternal blood sample on a flexible membrane analyzed for the presence of nuclei and hemoglobin. FIG. 20A shows an image of a cell nuclei on a membrane under fluorescent illumination to detect stained nuclei. FIG. 20B shows another cell on a membrane. The cell has been analyzed using immunocytochemistry using a mix anti-epsilon hemoglobin antibody and anti-embryonic hemoglobin antibody. The cell is positive for both fetal hemoglobin 260 and nuclei 262 and represents a candidate fetal cell.

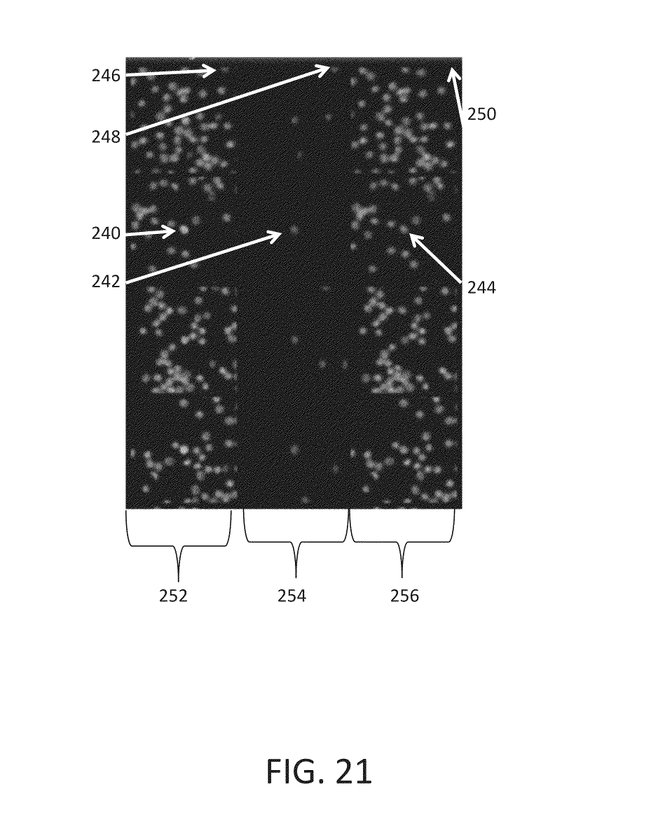

FIG. 21 shows portions of a self-assembling monolayer from a maternal blood sample analyzed using 420 nm light for hemoglobin (256), fluorescence for nuclei (254), and both (252). Concurrence of hemoglobin and nuclei signals would indicate a nucleated red blood cell (potential fetal red blood cell). Fluorescent image 246 and fluorescent image 248 indicate a first cell is nucleated while blank spot 250 indicates the cell lacks hemoglobin. The cell is a nucleated, non-hemoglobin containing cell such as a white blood cell. Fluorescent image 242 and 420 nm signal 244, which overlap in both channels to form hybrid signal 240, indicate that a second cell is a nucleated red blood cell (a potential fetal red blood cell).

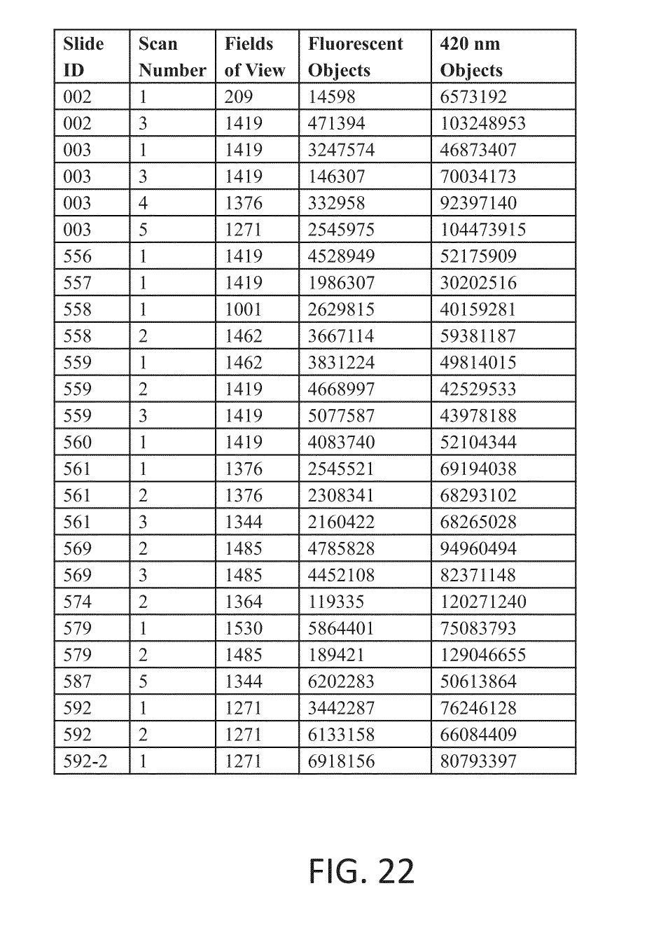

FIG. 22 shows an initial result of various monolayer analyses using an automated analysis system according to the disclosure. The fields of view (FOV) show the number of fields of view that were analyzed from a monolayer. The number of fluorescent objects found which can be further analyzed to identify potential nucleated cells (white blood cells and nucleated red blood cells) is indicated. The number of 420 nm positive objects (but without a nuclear signal) which would be indicate non-nucleated red blood cells, are indicated.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of separating a candidate fetal cell from a maternal blood sample comprising:
placing a sample of maternal blood enriched for fetal cells on a surface wherein the sample comprises maternal nucleated cells and a fetal cell;
removably attaching at least a first portion of the cells in the sample to the surface;
performing an analysis on the cells to identify a candidate fetal nucleated cell; and
removing the candidate fetal nucleated cell from the surface to thereby isolate the cell from the remainder of the cells.

2. The method of claim 1 wherein removably attaching comprises loosely adhering the cells to the surface such that a cell may be removed intact from the surface using a micropipette and a partial vacuum.

3. The method of claim 1 further comprising adding a material wherein the material is configured to reduce cell adhesion to the surface.

4. The method of claim 1 further comprising adding bovine serum albumin prior to the removably attaching step.

5. The method of claim 1 further comprising maintaining a second portion of cells close to the surface.

6. The method of claim 1 wherein placing a sample of maternal blood on the surface comprises placing viable cells on the surface, the method further comprising maintaining cell viability at least through the removably attaching step.

7. The method of claim 1 wherein removing a candidate fetal nucleated cell comprises subjecting the cell to a partial vacuum to thereby remove the candidate fetal nucleated cell.

8. The method of claim 1 wherein removing a candidate fetal nucleated cell comprises gathering the cell in a micropipette having an inner diameter less than 30 μm.

9. The method of claim 1 wherein removing a candidate fetal nucleated cell comprises gathering the cell in a micropipette coated with bovine serum albumin (BSA).

10. The method of claim 1 wherein providing comprises providing a transparent, rigid surface.

11. The method of claim 1 wherein providing comprises providing a functionalized surface on a transparent backing.

12. A method for attaching a fetal cell to a surface comprising:
placing a solution configured to maintain fetal cell integrity on a porous membrane;
placing a potential fetal cell in the solution on the membrane; and
removing the solution and thereby adhering the potential fetal cell to the porous membrane.

13. The method of claim 12 wherein removing the solution comprises evaporating the solution.

14. The method of claim 12 wherein placing comprises placing the solution on a porous polyester membrane.

15. The method of claim 12 wherein placing comprises placing the solution on a membrane comprising one of polyethylene terephthalate (PET) and polyethylene naphthlate (PEN).

16. The method of claim 12 wherein placing a solution comprises placing a solution volume between about 50 fl and about 10 ul.

17. The method of claim 12 further comprising repeating the placing a solution, placing a potential fetal cell, and the removing the solution steps to thereby attach a plurality of cells with the membrane wherein each cell is separated from any other cell on the membrane.

18. The method of claim 12 further comprising having the porous membrane in a controlled environment comprising less than 100% humidity during at least a part of the removing step.
